SEQUENCE LISTING (57 sequences)

SEQ ID NO: 1 - protein, 28 aa, Artificial Sequence
  Chemically synthesized - Part of tpA
RXEXXXXXXXXXXXXXRXXXXXXXXEXR

SEQ ID NO: 2 - protein, 8 aa, Artificial Sequence
  Chemically synthesized - Part of fibronectin
RXEXKXER

SEQ ID NO: 3 - protein, 40 aa, Artificial Sequence
  Chemically synthesized - Peptide A Beta (1-40)
DAEFRHDSGYEVHHQKLVFFAEDVGSNKGAIIGLMVGGVV

SEQ ID NO: 4 - protein, 13 aa, Artificial Sequence
  Chemically synthesized - Peptide FP13
KRLEVDIDIKIRS

SEQ ID NO: 5 - protein, 12 aa, Artificial Sequence
  Chemically synthesized - Peptide FP12
KRLEVDIDIKIR

SEQ ID NO: 6 - protein, 10 aa, Artificial Sequence
  Chemically synthesized - Peptide FP10
KRLEVDIDIK

SEQ ID NO: 7 - protein, 37 aa, Artificial Sequence [sequence omitted]

SEQ ID NO: 8 - protein, 10 aa, Artificial Sequence
  Chemcially synthesized - Peptide Delta hIAPP
SNNFGAILSS

SEQ ID NO: 9 - protein, 10 aa, Artificial Sequence
  Chemically synthesized - Peptide Delta mIAPP
SNNLGPVLPP

SEQ ID NO: 10 - DNA, 38 bp, Artificial Sequence
  Chemically synthesized - Primer
aaaagtcgacagccgccaccatggatgcaatgaagaga

SEQ ID NO: 11 - DNA, 33 bp, Artificial Sequence
  Chemically synthesized - Primer
gagtcacggacagttttcacccgccggcgaaaa

SEQ ID NO: 12 - DNA, 32 bp, Artificial Sequence
  Chemically synthesized - Primer
ctttatctatggtcccggtgcgccggcgaaaa

SEQ ID NO: 13 - DNA, 29 bp, Artificial Sequence
  Chemically synthesized - Primer
aaaagagatctgtgcctgtcaaaagttgc

SEQ ID NO: 14 - DNA, 32 bp, Artificial Sequence
  Chemically synthesized - Primer
aaaagagatctgataccagggccacgtgctac

SEQ ID NO: 15 - DNA, 35 bp, Artificial Sequence
  Chemically synthesized - Primer
acgagactccctttgtcactgcccgccggcgaaaa

SEQ ID NO: 16 - DNA, 31 bp, Artificial Sequence
  Chemically synthesized - Primer
aaaagcggccgcctggctcctcttctgaatc

SEQ ID NO: 17 - DNA, 31 bp, Artificial Sequence
  Chemically synthesized - Primer
tgcaagatctatagctgagaagtgttttgat

SEQ ID NO: 18 - DNA, 35 bp, Artificial Sequence
  Chemically synthesized - Primer
gtgaacgtgaagatccttatgtcccgccggcgtag

SEQ ID NO: 19 - DNA, 31 bp, Artificial Sequence
  Chemically synthesized - Primer
tgcaagatctacttctagaaatagatgcaac

SEQ ID NO: 20 - DNA, 32 bp, Artificial Sequence
  Chemically synthesized - Primer
ctctccgtgtggagacaccccgccggcgtagt

SEQ ID NO: 21 - DNA, 31 bp, Artificial Sequence
  Chemically synthesized - Primer
aaaaaagatctaaccaacctacggatgactc

SEQ ID NO: 22 - DNA, 32 bp, Artificial Sequence
  Chemically synthesized - Primer
tggacccccacttgggtcagccatggaaaaaa

SEQ ID NO: 23 - DNA, 32 bp, Artificial Sequence
  Chemically synthesized - Primer
gaaacaagatctcagaaagagaagtgctttga

SEQ ID NO: 24 - DNA, 34 bp, Artificial Sequence
  Chemically synthesized - Primer
tcgccgaccggtcggtccggcccgccggcgggca

SEQ ID NO: 25 - DNA, 31 bp, Artificial Sequence
  Chemically synthesized - Primer
aaaaaagatctcagaaagagaagtgctttga

SEQ ID NO: 26 - DNA, 31 bp, Artificial Sequence
  Chemically synthesized - Primer
gcacctgtggttccgttcggccatggaaaaa

SEQ ID NO: 27 - DNA, 32 bp, Artificial Sequence
  Chemically synthesized - Primer
gcaagaagatctggcacagagaaatgctttga

SEQ ID NO: 28 - DNA, 34 bp, Artificial Sequence
  Chemically synthesized - Primer
ttccgtgggctgtatgtcgacccgccggcgggaa

SEQ ID NO: 29 - DNA, 30 bp, Artificial Sequence
  Chemically synthesized - Primer
aaaaaagatctggcacagagaatgctttga

SEQ ID NO: 30 - DNA, 31 bp, Artificial Sequence
  Chemically synthesized - Primer
gttgtagctcggactactcgccatggaaaaa

SEQ ID NO: 31 - protein, 11 aa, Artificial Sequence
  Chemically synthesized - TTR11
YTIAALLSPYS

SEQ ID NO: 32 - protein, 12 aa, Artificial Sequence
  Chemically synthesized - LAM12
AASIKVAVSADR

SEQ ID NO: 33 - protein, 13 aa, Artificial Sequence
  Chemically synthesized - amyloid fragment with Lys157Ala mutation (FP13)
KRLEVDIDIAIRS

SEQ ID NO: 34 - DNA, 28 bp, Artificial Sequence
  Chemically synthesized - Forward primer
gagatctgctcaaaacatcacagcccgg

SEQ ID NO: 35 - DNA, 28 bp, Artificial Sequence
  Chemically synthesized - Reverse primer
gcggccgcctcgcctggttcgatgatgc

SEQ ID NO: 36 - protein, 50 aa, Artificial Sequence
  Chemically synthesized - Finger domain of tPA
SYQVICRDEKTQMIYQQHQSWLRPVLRSNRVEYCWCNSGRAQCHSVPVKS

SEQ ID NO: 37 - protein, 47 aa, Artificial Sequence
  Chemically synthesized - Finger domain of FXII
QKEKCFEPQLLRFFHKNEIWYRTEQAAVARCQCKGPDAHCQRLASQA

SEQ ID NO: 38 - protein, 47 aa, Artificial Sequence
  Chemically synthesized - Finger domain of HGFa
GTEKCFDETRYEYLEGGDRWARVRQGHVEQCECFGGRTWCEGTRHTA

SEQ ID NO: 39 - protein, 44 aa, Artificial Sequence
  Chemically synthesized - Finger domain of FN1-1
SKPGCYDNGKHYQINQQWERTYLGNVLVCTCYGGSRGFNCESKP

SEQ ID NO: 40 - protein, 49 aa, Artificial Sequence
  Chemically synthesized - Finger domain of FN1-2
EAEETCFDKYTGNTYRVGDTYERPKDSMIWDCTCIGAGRGRISCTIANR

SEQ ID NO: 41 - protein, 50 aa, Artificial Sequence
  Chemically synthesized - Finger domain of FN1-3
TIANRCHEGGQSYKIGDTWRRPHETGGYMLECVCLGNGKGEWTCKPIAEK

SEQ ID NO: 42 - protein, 51 aa, Artificial Sequence
  Chemically synthesized - Finger domain of FN1-4
KPIAEKCFDHAAGTSYVVGETWEKPYQGWMMVDCTCLGEGSGRITCTSRNR

SEQ ID NO: 43 - protein, 52 aa, Artificial Sequence
  Chemically synthesized - Finger domain of FN1-5
TSRNRCNDQDTRTSYRIGDTWSKKDNRGNLLQCICTGNGRGEWKCERHTSVQ

SEQ ID NO: 44 - protein, 45 aa, Artificial Sequence
  Chemically synthesized - Finger domain of FN1-6
PPYGHCVTDSGVVYSVGMQWLKTQGNKQMLCTCLGNGVSCQETAV

SEQ ID NO: 45 - protein, 56 aa, Artificial Sequence
  Chemically synthesized - Finger domain of FN1-7
PMAAHEEICTTNEGVMYRIGDQWDKQHDMGHMMRRCTCVGNGRGEWTCIAYSQLRD

SEQ ID NO: 46 - protein, 52 aa, Artificial Sequence
  Chemically synthesized - Finger domain of FN1-8
IAYSQLRDQCIVDDITYNVNDTFHKRHEEGHMLNCTCFGQGRGRWKCDPVDQ

SEQ ID NO: 47 - protein, 54 aa, Artificial Sequence
  Chemically synthesized - Finger domain of FN1-9
WKCDPVDQCQDSETGTFQIGDSWEKYVHGVRYQCYCYGRGIGEWHCQPLQTYPS

SEQ ID NO: 48 - protein, 53 aa, Artificial Sequence
  Chemically synthesized - Finger domain of FN1-10
NQPTDDSCFDPYTVSHYAVGDEWERMSSESGFKLLCQCLGFGSGHFRCDSSRW

SEQ ID NO: 49 - protein, 49 aa, Artificial Sequence
  Chemically synthesized - Finger domain of FN1-11
DSSRWCHDNGVNYKIGEKWDRQGENGQMMSCTCLGNGKGEFKCDPHEAT

SEQ ID NO: 50 - protein, 50 aa, Artificial Sequence
  Chemically synthesized - Finger domain of FN1-12
DPHEATCYDDGKTYHVGEQWQKEYLGAICSCTCFGGQRGWRCDNCRRPGG

SEQ ID NO: 51 - DNA, 32 bp, Artificial Sequence; identical to SEQ ID NO: 20
  Chemically synthesized - f25-primer (5)

SEQ ID NO: 52 - DNA, 31 bp, Artificial Sequence; identical to SEQ ID NO: 17
  Chemically synthesized - f25-primer (8)

SEQ ID NO: 53 - DNA, 50 bp, Artificial Sequence
  Chemically synthesized - f25-primer (9)
ctctccgtgtggagacacgtggtagtggtagtggtaatcgccggcggggg

SEQ ID NO: 54 - DNA, 28 bp, Artificial Sequence
  Chemically synthesized - f25-primer (10)
ggggagatcttaccaagtgatctgcaga

SEQ ID NO: 55 - DNA, 51 bp, Artificial Sequence
  Chemically synthesized - f25-primer (11)
gagtcacggacagttttcagtggtagtggtagtggtaatcgccggcggggg

SEQ ID NO: 56 - DNA, 31 bp, Artificial Sequence; identical to SEQ ID NO: 17
  Chemically synthesized - f25-primer (12)

SEQ ID NO: 57 - DNA, 50 bp, Artificial Sequence; identical to SEQ ID NO: 53
  Chemically synthesized - f25-primer (13)

(19) United States
(12) Patent Application Publication
(10) Pub. No.: US 2009/0202980 A1
(43) Pub. Date: Aug. 13, 2009

(54) Cross-Beta Structure Comprising Amyloid Binding Proteins and Methods for Detection of the Cross-Beta Structure, for Modulating Cross-Beta Structures Fibril Formation and for Modulating Cross-Beta Structure-Mediated Toxicity and Method for Interfering With Blood Coagulation

(75) Inventors: Martijn Frans Ben Gerard Gebbink, Eemnes (NL); Barend Bouma, Houten (NL); Onno Wouter Kranenburg, Amsterdam (NL); Louise Maria Johanna Kroon, Bunnik (NL)

Correspondence Address: TRASKBRITT, P.C., P.O. BOX 2550, SALT LAKE CITY, UT, 84110, US

(73) Assignee: Crossbeta Biosciences B.V., Utrecht (NL)

(21) Appl. No.: 11/886,867

(22) PCT Filed: Mar. 21, 2006
(86) PCT No.: PCT/NL2006/000149

§ 371 Date: Dec. 11, 2007

Related U.S. Application Data
(63) Continuation-in-part of application No. 11/087,102, filed on Mar. 21, 2005.

(51) Int. Cl. A01N 1/02; C07K 16/00

(52) U.S. Cl. 435/2; 530/387.3

(57) ABSTRACT

The invention relates to the field of biochemistry, molecular biology, structural biology and medicine. More in particular, the invention relates to cross-β structures and the biological role of these cross-β structures.

### CROSS-REFERENCE TO RELATED APPLICATIONS

This is a national phase entry under 35 U.S.C. § 371 of International Patent Application PCT/NL2006/000149, filed Mar. 21, 2006, published in English as International Patent Publication WO 2006/101387 A2 on Sep. 28, 2006, which claims the benefit under 35 U.S.C. § 119 of U.S. patent application Ser. No. 11/087,102, filed Mar. 21, 2005, pending. This application also claims priority to U.S. patent application Ser. No. 11/033,105 filed Jan. 10, 2005; European Patent Application No. 02077797.5 filed Jul. 9, 2002; and International Patent Application PCT/NL2003/000501 filed Jul. 8, 2003.

### TECHNICAL FIELD

The invention relates to the field of biochemistry, molecular biology, structural biology and medicine. In particular, the invention relates to cross-β structures, their binding proteins and their biological roles.

### BACKGROUND

An increasing body of evidence suggests that unfolding of globular proteins can lead to toxicity. Unfolded proteins can initiate protein misfolding, aggregation and fibrillization by adopting a partially structured conformation. Such (fibrillar) aggregates can (slowly) accumulate in various tissue types and are associated with a variety of degenerative diseases. The term “amyloid” is used to describe these fibrillar deposits (or plaques). Diseases characterized by amyloid are referred to as amyloidosis and include Alzheimer disease (AD), light-chain amyloidosis, type II diabetes and (transmissible) spongiform encephalopathies. It has been found recently that toxicity is an inherent property of misfolded proteins. According to the present invention, there is a common mechanism for these conformational diseases.

A cross-β structure is a secondary/tertiary/quaternary structural element in peptides or proteins. A cross-β structure (also referred to as a “cross beta structure,” a “crossbeta structure,” a “cross-beta structure” or a “cbs”) is defined as a protein or peptide or a part of a protein or peptide, or a part of an assembly of peptides and/or proteins, which comprises single β-strands (stage 1) and/or a(n ordered) group of β-strands (stage 2), and/or a group of β-strands arranged in a β-sheet (stage 3), and/or a group of stacked β-sheets (stage 4). A cross beta structure is also referred to as an “amyloid.”

A crossbeta structure precursor is defined as a protein conformation that precedes the formation of any of the aforementioned structural stages of a crossbeta structure. Non-limiting examples of peptides with crossbeta structure precursor conformation are human fibrin α-chain fragments, yeast prion protein Sup32 fragment and human amyloid-β peptides.

A typical form of stacked β-sheets is in a fibril-like structure in which the β-sheets are stacked in either the direction of the axis of the fibril or perpendicular to the direction of the axis of the fibril. The direction of the stacking of the β-sheets in cross-β structures is perpendicular to the long fiber axis.

A typical form of a crossbeta structure precursor is a partially or completely misfolded protein, a partially or completely unfolded protein, a partially or completely refolded protein, a partially or completely aggregated protein, an oligomerized or multimerized protein, or a partially or completely denatured protein.

Crossbeta structures and crossbeta structure precursors appear as monomeric molecules or dimeric, trimeric, through oligomeric assemblies of molecules, and appear as multimeric structures and/or assemblies of molecules. Crossbeta structures (precursor) in any state, from monomeric molecules through a multimeric assembly of molecules, can appear in soluble form in aqueous solutions and/or organic solvents and/or any other solutions like, for example, soluble oligomers, and/or crossbeta structures, in any state, from monomeric molecules through a multimeric assembly of molecules, can be present as solid state materials in solutions like, for example, insoluble aggregates, fibrils, or particles like, for example, a suspension, or separated in a solid crossbeta structure phase and a solvent phase.

Soluble crossbeta structure or crossbeta structure precursor is defined as the fraction of molecules that are present in a solution after applying 100,000*g to the solution for one hour. A cross-β structure conformation is a signal that triggers a cascade of events that induces clearance and breakdown of the obsolete protein or peptide. When clearance is inadequate, unwanted proteins and/or peptides aggregate and form toxic structures, ranging from soluble oligomers up to precipitating fibrils and amorphous plaques. Such cross-β structure conformation-comprising aggregates underlie various diseases, such as, for instance, Huntington's disease, amyloidosis-type disease, atherosclerosis, diabetes, asthma, colitis, Crohn's disease, infections, bleeding, thrombosis, cancer, sepsis, inflammatory diseases, rheumatoid arthritis, transmissible spongiform encephalopathies such as Creutzfeldt-Jakob disease, multiple sclerosis, auto-immune diseases, diseases associated with loss of memory such as Alzheimer's disease, Parkinson's disease and other neuronal diseases (epilepsy), encephalopathy, encephalitis, cataract and systemic amyloidoses.

The term “peptide” is intended to include oligopeptides as well as polypeptides, and the term “protein” includes proteinaceous molecules with and without post-translational modifications, such as glycosylation and glycation. It also includes lipoproteins and complexes comprising a proteinaceous part, such as protein-nucleic acid complexes (RNA and/or DNA), membrane-protein complexes, etc. As used herein, the term “protein” also encompasses proteinaceous molecules, peptides, oligopeptides and polypeptides.

Cross-β structure can, for example, be formed upon denaturation, proteolysis, misfolding or unfolding of proteins. This structure element is typically absent in globular regions of proteins. A cross-β structure is, for instance, formed during unfolding and refolding of proteins and peptides. Unfolding of proteins occur regularly within an organism. For instance, proteins often unfold and refold spontaneously during intracellular protein synthesis and/or during and/or at the end of their life cycle. Moreover, unfolding and/or refolding is induced by environmental factors, such as, for instance, pH, glycation, (partial) de-glycosylation, oxidative stress, heat, irradiation, mechanical stress, proteolysis and so on. The terms “unfolding,” “refolding” and “misfolding” relate to the three-dimensional structure of a protein. Unfolding means that a protein loses at least part of its three-dimensional structure. The term “refolding” relates to at least partly coiling back into some kind of three-dimensional structure. By refolding, a protein can regain its native configuration or an incorrect refolding can occur. The term “incorrect refolding” refers to a situation when a three-dimensional structure other than a native configuration is formed. Incorrect refolding is also called misfolding. Unfolding and refolding of proteins involve the risk of cross-β structure formation. Formation of cross-β structure sometimes also occurs directly after protein synthesis, without a correctly folded protein intermediate. The cross-β structure is, for instance, found in amyloid fibrils. Amyloid peptides or proteins are cytotoxic to cells.

### DISCLOSURE OF THE INVENTION

Glycation of proteins and exposure of protein to lipids and/or non-self substances also induces formation of cross-β structure, which is reported herein. The results indicate that a common structure is induced upon misfolding of globular proteins. Therefore, the present invention discloses a novel pathway involving cross-β structure, which pathway will be called “cross-β structure pathway” or “cross-β pathway.” The cross-β pathway is involved in, amongst other biological activities, clearance, breakdown and neutralization of obsolete proteins. This pathway consists of several cross-β structure-binding proteins, including so-called multiligand receptors, and is involved in, amongst other biological activities, protein degradation and/or protein clearance and/or neutralization of toxic protein conformations. Inadequate functioning of this pathway, for example, results in the development of diseases, such as conformational diseases and/or amyloidosis. Also reported herein is the identification of novel cross-β structure-binding proteins that contain a cross-β structure-binding module. These findings support the identification of a cross-β structure pathway. Multiple aspects of this novel pathway are outlined below.

For example, the present invention discloses that proteolyzed, denatured, unfolded, misfolded, glycated, oxidized, acetylated or otherwise structurally altered proteins adopt cross-β structure. Examples of known cross-β structure-forming proteins are all proteins that cause amyloidosis or proteins that are found in disease-related amyloid depositions, for example, but not restricted to, Alzheimer amyloid-β peptide (Aβ) and Islet Amyloid PolyPeptide (IAPP). The present invention discloses that fibrin, glycated proteins (for example, glycated albumin and glycated hemoglobin) and endostatin are also capable of adopting cross-β structure.

The invention furthermore discloses the identification of the formation of a cross-β structure as a signal for protein degradation and/or protein clearance.

The serine protease tissue-type plasminogen activator (tPA) induces the formation of plasmin through cleavage of plasminogen. Plasmin cleaves fibrin and this occurs during lysis of a blood clot. tPA has been recognized for its role in fibrinolysis for a long time. Activation of plasminogen by tPA is stimulated by fibrin or fibrin fragments, but not by its precursor, fibrinogen. This can be in part explained by the strong binding of tPA to fibrin and weak binding to fibrinogen. Although the binding sites in fibrin and in tPA responsible for binding and activation of tPA have been mapped and studied in detail, the exact structural basis for the interaction of tPA with fibrin was unknown. In addition to fibrin and fibrin fragments, many other proteins have been described that are similarly capable of binding tPA and stimulating tPA-mediated plasmin formation. Like with fibrin and fibrin fragments, the exact nature of the interaction(s) between these ligands for tPA and tPA were not known. Moreover, it was unknown why and how all these proteins, which lack primary sequence homology, bind tPA. The invention now discloses tPA as a protein capable of binding cross-β structures. Furthermore, the invention discloses the finger domain (also named fibronectin type I domain) and other comparable finger domains as a cross-β structure-binding module. The present invention further discloses that proteins that bind to these fingers will be typically capable of forming cross-β structures.

Since fibrin contains cross-β structure, the present invention also discloses that the generation of cross-β structures plays a role in physiological processes.

The present invention furthermore discloses that the cross-β structure is a common denominator in ligands for multiligand receptors. The invention discloses, therefore, that multiligand receptors belong to the “cross-β pathway.”

The best studied example of a receptor for cross-β structure is a receptor for advanced glycation end products (RAGE). Examples of ligands for RAGE are Aβ, protein-advanced glycation end products (AGE) adducts (including glycated-bovine serum albumin (BSA)), IAPP, prion, amphoterin and S100. RAGE is a member of a larger family of multiligand receptors that includes several other receptors, some of which, including CD36, are known to bind cross-β structure-containing proteins (see also FIG. 1). At present, it is not clear what the exact nature of the structure or structures is in the ligands of these receptors that mediates the binding to these receptors.

It is reported herein that glycation of proteins also induces the formation of cross-β structure. Therefore, it is disclosed that all these receptors form part of a mechanism to deal with the destruction and removal of unwanted or even damaging proteins or agents. These receptors play a role in recognition of infectious agents or cells, in recognition of apoptotic cells and in internalization of protein complexes and/or pathogens. It is furthermore disclosed that all these receptors recognize the same or similar structure, the cross-β structure, to respond to undesired molecules.

It is shown that tPA binds cross-β structure, providing evidence that tPA belongs to the multiligand receptor family. As disclosed herein, tPA and the other multiligand receptors bind the cross-β structure and participate in the destruction of unwanted biomolecules. A prominent role of the protease tPA in the pathway lies in its ability to initiate a proteolytic cascade that includes the formation of plasmin. Proteolysis is likely to be essential for the degradation and subsequent removal of extracellular matrix components. The effect of tPA on the extracellular matrix will affect cell adhesion, cell migration, cell survival and cell death through, for example, integrin-mediated processes. Based on the studies herein, strong evidence is provided that at least three other proteins, factor XII a.k.a. Hageman factor (fXII), hepatocyte growth factor activator (HGFA) and fibronectin, that contain one or more finger domain(s), are also part of the “cross-β structure pathway.”

The role of FXII is especially important, since it activates the intrinsic coagulation pathway. Activation of the intrinsic pathway, and the resulting formation of vasoactive peptides, and the activation of other important proteins, contribute to the process of protection and/or clearance of undesired proteins or agents.

The “cross-β structure pathway” is modulated in many ways. Factors that regulate the pathway include modulators of synthesis and secretion, as well as modulators of activity. The pathway is involved in many physiological and pathological processes. Therefore, the invention furthermore provides a method for modulating extracellular protein degradation and/or protein clearance comprising modulating the activity of a receptor for cross-β structure-forming proteins. Examples of receptors for cross-β structure-forming proteins include RAGE, CD36, Low-density lipoprotein-Related Protein (LRP), Scavenger Receptor B-1 (SR-BI), and SR-A (see Tables 4 and 5 for more examples of crossbeta structure-binding receptors).

The invention discloses that FXII, HGFA and fibronectin are also receptors for cross-β structure, as well as Immune Globulin Intravenous (IgIV) and chaperones like, for example, clusterin, haptoglobin, gp96, BiP, other extracellularly located heat-shock proteins, proteases like, for example, hepatocyte growth factor activator, plasminogen, antibodies like, for example, the immunoglobulin G type and immunoglobulin M type, and cell surface receptors like, for example, low-density lipoprotein-related protein, CD36, CD91, scavenger receptor A, scavenger receptor B-I, and receptor for advanced glycation end products.

Immune Globulin Intravenous (IgIV) are immunoglobulins of apparently healthy animals or humans that are collected from serum or blood. IgIV are prescribed to animals and humans that have a lack of antibodies and/or to humans suffering from one or more of approximately 200 pathological conditions ranging from infections, amyloidosis, autoimmune diseases and inflammatory diseases.

Molecular chaperones are a diverse class of proteins comprising heat-shock proteins, chaperonins, chaperokines and stress proteins that are contributing to one of the most important cell defense mechanisms that not only facilitate protein folding, refolding of partially denatured proteins, protein transport across membranes, cytoskeletal organization, degradation of disabled proteins, and apoptosis, but also act as cytoprotective factors against deleterious environmental stresses. Individual members of the family of these specialized proteins bind non-native states of one or several, or a whole series of, classes of proteins and assist them in reaching a correctly folded and functional conformation. Alternatively, when the native fold cannot be achieved, molecular chaperones contribute to the effective removal of misfolded proteins by directing them to the suitable proteolytic degradation pathways. Chaperones selectively bind to non-natively folded proteins in a stable non-covalent manner. To direct correct folding of a protein from a misfolded form to the required native conformation, several chaperones mostly work together in consecutive steps.

Chaperonins are molecular machines that facilitate protein folding by undergoing energy (ATP)-dependent movements that are coordinated in time and space by complex allosteric regulation. Examples of chaperones that facilitate refolding of proteins from a misfolded conformation to a native form are heat-shock protein (hsp) 90, hsp60 and hsp70. Chaperones also participate in the stabilization of unstable protein conformers and in the recovery of proteins from aggregates. Molecular chaperones are mostly heat- or stress-induced proteins (hsps) that perform critical functions in maintaining cell homeostasis or are transiently present and active in regular protein synthesis. Hsps are among the most abundant intracellular proteins. Chaperones that act in an ATP-independent manner are the intracellular small hsps, calreticulin, calnexin, extracellular clusterin and haptoglobin. Under stress conditions such as elevated temperature, glucose deprivation and oxidation, small hsps and clusterin efficiently prevent the aggregation of target proteins. Interestingly, both types of hsps can hardly chaperone a misfolded protein to refold back to its native state. In patients with Creutzfeldt-Jakob, Alzheimer's disease and other diseases related to protein misfolding and accumulation of amyloid, increased expression of clusterin and small hsps has been seen. Molecular chaperones are essential components of the quality control machineries present in cells. Due to the fact that they aid in the folding and maintenance of newly translated proteins, as well as in facilitating the degradation of misfolded and destabilized proteins, chaperones are essentially the cellular sensors of protein misfolding and function. Chaperones are, therefore, the gatekeepers in a first line of defense against deleterious effects of misfolded proteins, by assisting a protein in obtaining its native fold or by directing incorrectly folded proteins to a proteolytic breakdown pathway.

The present invention further discloses that tPA is a cross-β structure-binding protein, a multiligand receptor and a member of the “cross-β structure pathway.” The invention discloses that tPA mediates cross-β structure-induced cell dysfunction and/or cell toxicity. The invention discloses that tPA mediates, at least in part, cell dysfunction and/or toxicity through activation of plasminogen. The plasminogen-dependent effects are inhibited by B-type carboxypeptidase activity and thereby a role for carboxyterminal lysine residues in the cross-β pathway is disclosed.

The present invention relates, amongst others, to the structure(s) in fibrin and other proteins that bind tPA to the binding domain in tPA and to the pathway(s) regulated by this structure. The present invention discloses a presence of cross-β structures in proteins and peptides that are capable of binding tPA. The herein-disclosed results indicate a strong correlation between the presence of a cross-β structure and the ability of a molecule to bind tPA. Furthermore, the results indicate the presence of an amyloid structure in fibrin. This indicates that under physiological conditions, a cross-β structure can form a phenomenon that has been previously unrecognized. The formation of cross-β structure has thus far only been associated with severe pathological disorders. tPA binds denatured proteins, which indicates that a large number of proteins, if not all proteins, can adopt a conformation-containing cross-β structure or cross-β structure-like structure(s). Taken together, the formation of cross-β structures is likely to initiate and/or participate in a physiological cascade of events necessary to adequately deal with removal of unwanted molecules, i.e., misfolded proteins, apoptotic cells or even pathogens. FIG. 1 shows a schematic representation of the “cross-β structure pathway.” This pathway regulates the removal of unwanted biomolecules during several processes, including fibrinolysis, formation of neuronal synaptic networks, clearance of used, unwanted, misfolded and/or destroyed (denatured) proteins, induction of apoptosis and clearance of apoptotic cells, necrotic cells and pathogens. If insufficiently or incorrectly regulated or dysbalanced, the pathway may lead to severe disease.

Thus, in a first embodiment, the invention provides a method for modulating extracellular protein degradation and/or protein clearance comprising modulating cross-β structure formation (and/or cross-β structure-mediated activity) of the protein present in the circulation.

There are two major regular protein-folding patterns, which are known as the β-sheet and the α-helix. An antiparallel β-sheet is formed when an extended polypeptide chain folds back and forth upon itself, with each section of the chains running in the direction opposite to that of its immediate neighbors. This results in a structure held together by hydrogen bonds that connect the peptide bonds in neighboring chains. Regions of a polypeptide chain that run in the same direction form a parallel β-sheet. A cross-β structure is composed of stacked β-sheets. In a cross-β structure, the individual β-strands run either perpendicular to the long axis of a fibril or the β-strands run in parallel to the long axis of a fiber. The direction of the stacking of the β-sheets in cross-β structures is perpendicular to the long fiber axis. As disclosed herein within the experimental part, a broad range of proteins is capable of adopting cross-β structure and, moreover, these cross-β structure-comprising proteins are all capable of binding and stimulating tPA and thereby promoting destruction of unwanted or damaging proteins or agents.

An extracellular protein is typically defined as a protein present outside a cell or cells.

Protein degradation and/or protein clearance and/or protein neutralization includes the breakdown, removal and/or coverage/shielding of unwanted proteins, for example, unwanted and/or destroyed (for example, denatured and/or misfolded) protein. Also included is the removal of unwanted biomolecules during several processes, including fibrinolysis, formation of neuronal synaptic networks, clearance of used, unwanted and/or destroyed (denatured) proteins, induction of apoptosis and clearance of apoptotic cells, necrotic cells and pathogens.

The term “in the circulation” is herein defined as circulation outside a cell or cells, for example, but not restricted to, the continuous movement of blood.

It is possible to degrade and/or remove a protein that does not comprise a cross-β structure. This is, for example, accomplished by providing a compound comprising a cross-β structure and a compound comprising tPA-like activity at or near the protein that needs to be degraded and/or removed. An example of a compound comprising a cross-β structure is fibrin or a fragment thereof comprising the cross-β structure. An example of a compound comprising tPA-like activity is tPA.

In another embodiment, the invention provides a method for decreasing extracellular protein degradation and/or protein clearance comprising decreasing cross-β structure formation of the protein present in the circulation. More preferably, the invention provides a method for decreasing extracellular protein degradation and/or protein clearance comprising providing a compound capable of decreasing cross-β structure formation of the protein present in the circulation. Decreasing of cross-β structure formation is, for example, accomplished by shielding or blocking of the groups involved in the formation of cross-β structure. Examples of compounds capable of decreasing cross-β structure formation are Congo red, antibodies, β-breakers, phosphonates, heparin, amino-guanidine, laminin, IgIV, chaperones like, for example, clusterin, haptoglobin, gp96, BiP, other extracellularly located heat-shock proteins, proteases like, for example, HGFA, antibodies like, for example, the immunoglobulin G type and immunoglobulin M type, and (soluble extracellular fragments of) cell surface receptors like, for example, CD36, CD91, SRA, SRB-I and RAGE, and a cross beta structure-binding domain of any of the cross beta structure-binding compounds tabulated in Tables 3, 4 and 5. Yet another way to decrease cross-β structure formation in a protein is by removal of a glucose group involved in the glycation of the protein.

In yet another embodiment, the invention provides a method for modulating extracellular protein degradation and/or protein clearance comprising modulating tPA or tPA-like activity. tPA induces the formation of plasmin through cleavage of plasminogen. Plasmin cleaves fibrin and this occurs during lysis of a blood clot. Activation of plasminogen by tPA is stimulated by fibrin or fibrin fragments, but not by its precursor fibrinogen. The term “tPA-like activity” is herein defined as a compound capable of inducing the formation of plasmin, possibly in different amounts, and/or other tPA-mediated activities like, for example, binding to a protein comprising crossbeta structure. Preferably, tPA-like activity is modified such that it has a higher activity or affinity towards its substrate and/or a cofactor. This is, for example, accomplished by providing the tPA-like activity with multiple binding domains for cross-β structure-comprising proteins. Preferably, the tPA-like activity is provided with multiple finger domains. It is herein disclosed that the three-dimensional structures of the tPA finger domain and the fibronectin finger domains 4-5 reveal striking structural homology with respect to local charge-density distribution. Both structures contain a similar-solvent-exposed stretch of five amino acid residues with alternating charge; for tPA, Arg7, Glu9, Arg23, Glu32, Arg30, and for fibronectin, Arg83, Glu85, Lys87, Glu89, Arg90, located at the fifth finger domain, respectively. The charged-residue alignments are located at the same side of the finger module. Hence, preferably, the tPA-like activity is provided with one or more additional finger domain(s) that comprise(s) ArgXGlu(X)₁₃Arg(X)₈GluXArg (SEQ ID NO:1) or ArgXGluXLysXGluArg (SEQ ID NO:2).

The activity of tPA and/or the tPA-mediated activation of plasminogen are increased by the binding to fibrin fragments, or other protein fragments that have a lysine or an arginine at the carboxy-terminal end. B-type carboxypeptidases including, but not limited to, carboxypeptidase B (CpB) or Thrombin Activatable Fibrinolysis Inhibitor (TAFI, also named carboxypeptidase U or carboxypeptidase R), are enzymes that cleave off carboxy-terminal lysine and arginine residues of fibrin fragments that would otherwise bind to tPA and/or plasminogen and stimulate plasmin formation.

In a preferred embodiment, the invention provides a method for increasing extracellular protein degradation and/or protein clearance comprising providing a compound capable of increasing tPA-like and/or tPA-mediated activity or activities. In an even more preferred embodiment, the invention provides a method for increasing extracellular protein degradation and/or protein clearance comprising providing a compound capable of increasing tPA-like activity, wherein the compound comprises a cross-β structure. In another embodiment, the invention provides a method for increasing extracellular protein degradation and/or protein clearance comprising providing a compound capable of inhibiting B-type carboxypeptidase activity. In a more preferred embodiment, the compound comprises carboxypeptidase inhibitor (CPI) activity.

In yet another embodiment, the invention provides a method for decreasing extracellular protein degradation and/or protein clearance comprising providing a compound capable of decreasing tPA-like activity. More preferably, the invention provides a method for decreasing extracellular protein degradation and/or protein clearance comprising providing a compound capable of decreasing tPA-like activity or tPA-mediated activity or activities, wherein the compound is, a protein and/or a functional equivalent and/or a functional fragment thereof. For example, such a compound capable of decreasing tPA-like activity is an inhibitor of tPA or a substrate of tPA that binds and does not let go. Examples of a compound capable of decreasing tPA-like activity or tPA-mediated activity include, but are not limited to, lysine, arginine, ε-amino-caproic acid or tranexamic acid, serpins (for example, neuroserpin, PAI-1), tPA-Pevabloc, antibodies that inhibit tPA-like activity or tPA-mediated activity, B-type carboxypeptidase(s), and cross beta structure-binding domains of the compounds listed in Tables 3-5. For example, providing lysine results in the prevention or inhibition of binding of a protein comprising a C-terminal lysine-residue to the Kringle domain of plasminogen. Hence, tPA activation is prevented or inhibited. Preferably, the compound capable of decreasing tPA-like activity or tPA-mediated activity or activities reduce the tPA-like activity or tPA-mediated activity or activities and even more preferably, the tPA-like activity or tPA-mediated activity or activities is completely abolished.

A functional fragment and/or a functional equivalent are typically defined as a fragment and/or an equivalent capable of performing the same function, possibly in different amounts. A functional fragment of an antibody capable of binding to a cross-β structure would, for example, comprise the Fab′ fragment of the antibody.

In yet another embodiment, the invention provides a method for modulating extracellular protein degradation and/or protein clearance comprising modulating an interaction between a compound comprising a cross-β structure and a compound comprising tPA-like activity. In another embodiment, the invention provides a method for decreasing extracellular protein degradation and/or protein clearance comprising decreasing an interaction between a compound comprising a cross-β structure and a compound comprising tPA-like activity. Such a compound is, for example, a chemical, a proteinaceous substance or a combination thereof.

In a more preferred embodiment, the invention provides a method for decreasing extracellular protein degradation and/or protein clearance comprising providing a compound capable of decreasing an interaction between a compound comprising a cross-β structure and a compound comprising tPA-like activity. Even more preferably, the invention provides a method for decreasing extracellular protein degradation and/or protein clearance according to the invention, wherein the compound is a protein and/or a functional equivalent and/or a functional fragment thereof. Even more preferably, the protein is an antibody and/or a functional equivalent and/or a functional fragment thereof. Another example is Congo red.

The invention also provides a method for decreasing extracellular protein degradation and/or protein clearance comprising decreasing an interaction between a compound comprising a cross-β structure and a compound comprising tPA-like activity, wherein the interaction is decreased by providing a compound capable of competing with the interaction. Preferably, the compound capable of competing with the interaction comprises a finger domain and even more preferably, the finger domain comprises a stretch of at least five amino acid residues with alternating charge, for example, ArgXGlu(X)₁₃Arg(X)₈GluXArg (SEQ ID NO:1) or ArgXGluXLysXGluArg (SEQ ID NO:2). Preferably, the compound is fibronectin, FXII, HGFA, tPA, IgIV and/or a chaperone like, for example, clusterin, haptoglobin, gp96, BiP, other extracellularly located heat-shock proteins, antibodies like, for example, the immunoglobulin G type and immunoglobulin M type, and cell surface receptors like, for example, CD36, CD91, SRA, SRB-I, and RAGE.

It is clear that the invention also comprises a method for increasing extracellular protein degradation and/or protein clearance comprising increasing an interaction between a compound comprising a cross-β structure and a compound comprising tPA-like activity. This is, for example, accomplished by providing a compound capable of increasing an interaction between a compound comprising a cross-β structure and a compound comprising tPA-like activity. Preferably, the compound capable of increasing an interaction between a compound comprising a cross-β structure and a compound comprising tPA-like activity is a protein and/or a functional equivalent and/or a functional fragment thereof. For example, an antibody that stabilizes the interaction between a compound comprising cross-β structure and a compound comprising tPA-like activity, rendering the tPA-like activity in a continuous activated state, results in protein degradation and/or protein clearance to be increased. However, it is appreciated that increasing an interaction between a compound comprising a cross-β structure and a compound comprising tPA-like activity is also accomplished by mutations in either the compound comprising a cross-β structure or in the compound comprising tPA-like activity, like swapping of domains (for example, by providing the compound comprising tPA-like activity with other or more finger domains (obtainable from tPA, fibronectin, FXII or HGFA)), or by including binding domains of, for example, RAGE, CD36, IgIV and/or chaperones.

In yet another embodiment, the invention provides a method for modulating extracellular protein degradation and/or protein clearance comprising modulating an interaction of a compound comprising tPA-like activity and the substrate of the activity. It is clear that there are multiple ways by which the interaction can either be increased or decreased. An increase in the interaction between a compound comprising tPA-like activity and the substrate of the activity is, for example, accomplished by providing the compound comprising tPA-like activity with a mutation or mutations that improve the affinity of the compound with tPA-like activity for its substrate.

In yet another embodiment, the invention provides a method for removing cross-β structures from the circulation, using a compound comprising a cross-β structure-binding domain. Preferably, the compound is tPA or the finger domain of tPA. It is clear that a method of the invention also comprises a use of other cross-β structure-binding domains including, but not limited to, the finger domains of HGFA, FXII, fibronectin, IgIV and chaperones. It is clear that the invention also comprises antibodies that bind cross-β structures.

The present invention further discloses the use of a novel strategy to prevent the formation or to decrease/diminish (amyloid) plaques involved in a conformational disease, type II diabetes and/or ageing (e.g., Alzheimer's disease). Plaques are typically defined as extracellular fibrillar protein deposits (fibrillar aggregates) and are characteristic of degenerative diseases. The “native” properties of the constituent amyloid proteins may vary: some are soluble oligomers in vivo (e.g., transthyretin in familial amyloid polyneuropathy), whereas others are flexible peptides (e.g., amyloid-β in Alzheimer's disease (AD)). The basic pathogenesis of conformational diseases, for example, neurodegenerative disorders (AD, prion disorders), is thought to be related to abnormal pathologic protein conformation, i.e., the conversion of a normal cellular and/or circulating protein into an insoluble, aggregated, β-structure-rich form that is deposited in the brain. These deposits are toxic and produce neuronal dysfunction and death.

The formation of cross-β structures has thus far only been associated with severe pathological disorders. The results show that tPA and other receptors for cross-β structure-forming proteins can bind denatured proteins, indicating that a large number of proteins are capable of adopting a conformation-containing cross-β or cross-β-like structures. Taken together, the formation of a cross-β structure initiates or participates in a physiological cascade of events, necessary to adequately deal with removal of unwanted molecules; i.e., misfolded proteins, apoptotic cells or even pathogens. By increasing cross-β structure formation in a protein involved in a conformational disease, the pathway for protein degradation and/or protein clearance is activated and the protein is degraded, resulting in decreasing plaque or, more preferably, the plaque is completely removed. Hence, the effects of the conformational disease are diminished or more preferably completely abolished.

In another embodiment, the invention provides the use of a compound capable of binding to a cross-β structure for diminishing plaques and/or inhibiting cross-β structure-mediated toxicity involved in a conformational disease. In a preferable use of the invention, the compound is a protein and/or a functional equivalent and/or a functional fragment thereof and, even more preferably, the protein is tPA, a finger domain, IgIV, a chaperone like, for example, clusterin, haptoglobin, gp96, BiP, another extracellularly located heat-shock protein, a protease like, for example, HGFA, plasminogen, a cell surface receptor like, for example, CD36, CD91, SRA, SRB-I, and RAGE, an antibody and/or a functional equivalent and/or a functional fragment thereof. Examples of such antibodies are 4B5 or 3H7, and antibodies listed in Table 4.

In a further embodiment, the invention provides use of a compound capable of increasing tPA-like activity for diminishing plaques involved in a conformational disease. Preferably, the tPA-like activity is modified such that it has a higher activity or affinity towards its substrate and/or cofactor. This is, for example, accomplished by providing the tPA-like activity with multiple binding domains for cross-β structure-comprising proteins. Preferably, the binding domain comprises a finger domain and, even more preferably, the finger domain comprises a stretch of at least five amino acid residues with alternating charge, for example, ArgXGlu(X)₁₃Arg(X)₈GluXArg (SEQ ID NO:1) or ArgXGluXLysXGluArg (SEQ ID NO:2). Even more preferably, the finger domain is derived from fibronectin, FXII, HGFA, a chaperone, or tPA.

In yet another embodiment, the invention provides the use of a compound capable of binding to a cross-β structure for the removal of cross-β structures. Preferably, the compound is a protein and/or a functional equivalent and/or a functional fragment thereof. More preferably, the compound comprises tPA or tPA-like activity and/or a functional equivalent and/or a functional fragment thereof. Even more preferably, the functional fragment comprises a finger domain. Preferably, the finger domain comprises a stretch of at least five amino acid residues with alternating charge, for example, ArgXGlu(X)₁₃Arg(X)₈GluXArg (SEQ ID NO:1) or ArgXGluXLysXGluArg (SEQ ID NO:2). Even more preferably, the finger domain is derived from fibronectin, FXII, HGFA or tPA.

In another preferred embodiment, the protein is an antibody and/or a functional equivalent and/or a functional fragment thereof. In yet another preferred embodiment, the compound capable of specifically binding a cross-β structure comprises IgIv and/or a chaperone like, for example, clusterin, haptoglobin, gp96, BiP, another extracellularly located heat-shock protein, a protease like, for example, HGFA, plasminogen, and/or a cell surface receptor like, for example, CD36, CD91, SRA, SRB-I, and RAGE. With this use, the invention provides, for example, a therapeutic method to remove cross-β structure-comprising proteins from, for example, the circulation, preferably via extracorporeal dialysis. For example, a patient with sepsis is subjected to such use by dialysis of blood of the patient through means that is provided with, for example, preferably immobilized finger domains. One could, for example, couple the finger domains to a carrier or to the inside of the tubes used for dialysis. In this way, all cross-β structure-comprising proteins will be removed from the bloodstream of the patient, thereby relieving the patients of the negative effects caused by the cross-β structure-comprising proteins. Besides finger domain-comprising compounds, it is also possible to use other cross-β structure-binding compounds, like antibodies, Congo Red, IgIv and/or chaperones. It is also clear that the use could be applied in hemodialysis of patients suffering from renal insufficiency.

In yet another embodiment, the invention provides for the use of a compound capable of increasing or stabilizing an interaction of a compound comprising a cross-β structure and a compound comprising tPA-like activity for diminishing plaques involved in a conformational disease. Examples of a compound capable of increasing or stabilizing an interaction of a compound comprising a cross-β structure and a compound comprising tPA-like activity are given herein. Preferably, use according to the invention is provided, wherein the conformational disease is Alzheimer or diabetes. It is clear that the invention not only provides a use to decrease/diminish plaques involved in a conformational disease but that the onset of the disease can also be inhibited or more preferably completely prevented. Examples of diseases that can be prevented and/or treated according to the invention are conformational disease, amyloidosis-type diseases, atherosclerosis, diabetes, bleeding, thrombosis, cancer, sepsis and other inflammatory diseases, multiple sclerosis, auto-immune diseases, disease associated with loss of memory or Parkinson, and other neuronal diseases (epilepsy).

In another embodiment, the invention provides for the use of an antibody capable of recognizing a cross-β structure epitope for determining the presence of plaque involved in a conformational disease. In yet another embodiment, the invention provides for use of a cross-β structure-binding domain (preferably a finger domain from, for example, tPA) for determining the presence of a plaque and/or deposition involved in a conformational disease.

These uses of the invention provide a new diagnostic tool. It was not until the present invention that a universal cross-β-structure epitope was disclosed and that a diagnostic assay could be based on the presence of the cross-β structure. Such use is particularly useful for diagnostic identification of conformational diseases or diseases associated with amyloid formation, like Alzheimer or diabetes. It is clear that this diagnostic use is also useful for other diseases that involve cross-β structure formation, like all amyloidosis-type diseases, atherosclerosis, diabetes, bleeding, cancer, sepsis and other inflammatory diseases, multiple sclerosis, auto-immune diseases, disease associated with loss of memory or Parkinson, and other neuronal diseases (epilepsy). For example, one can use a finger domain (of, for example, tPA) and provide it with a label (radioactive, fluorescent, etc.). This labeled finger domain can then be used either in vitro or in vivo for the detection of cross-β structure-comprising proteins, hence, for determining the presence of a plaque and/or deposition involved in a conformational disease. One can, for example, use an ELISA assay to determine the amount of sepsis in a patient or one can localize a plaque involved in a conformational disease.

In yet another embodiment, the invention provides a recombinant tPA comprising an improved cross-β structure-binding domain or multiple cross-β structure-binding domains. Preferably, the tPA is provided with multiple, possibly different, finger domains. A recombinant tPA comprising an improved cross-β structure-binding domain or multiple cross-β structure-binding domains is used for different purposes, for example, in a method for the improved treatment of thrombosis or for the removal of cross-β stricture-comprising proteins from the circulation of a patient in need thereof. Another use of a recombinant tPA comprising an improved cross-β structure-binding domain or multiple cross-β structure-binding domains is in diagnostic assays, for example, in a BSE detection kit or in imaging experiments. Imaging with a recombinant tPA comprising an improved cross-β structure-binding domain or multiple cross-β structure-binding domains is, for example, useful for detection of apoptosis. For example, labeled tPA including, but not limited to, radio-labeled tPA, is inoculated in an individual, followed by detection and localization of labeled tPA in the body. It is clear that recombinant tPA comprising a cross-β structure-binding domain or multiple cross-β structure-binding domains are also useful in therapeutic applications.

This invention has made clear that the cross-β structure is harmful when present in certain parts of the body like, for example, the brain, and the damage is effected by the combination of cross-β structure with tPA. A method is provided to inhibit cross-β structure-mediated effects comprising providing an effective amount of IgIV, a chaperone and/or a protein comprising a finger domain to block the binding sites of the cross-β structure for tPA. The cross-β structure-mediated effects may even be further diminished comprising providing an effective amount of B-type carboxypeptidase activity to inhibit the tPA activity.

In another embodiment, the local cross-β structure-mediated effect can be used against tumors. To that effect, cross-β structure-mediated effects are locally induced to increase local cytotoxicity and/or fibrinolysis comprising locally administering an effective amount of cross-β structures and/or cross-β structure-inducing compounds in conjunction with tPA or a compound with tPA-like activity and/or CPI or a compound with CPI-like activity.

The present invention provides, in a further embodiment, a method according to the invention that is carried out ex vivo, e.g., by dialysis. According to this embodiment, the circulating fluid (blood) of a subject is sent into a system outside the body for clearing proteinaceous molecules comprising cross-β structure like, for example, β2-microglobulin, from the circulation. Preferably, such a system is a flow-through system connected to the body circulation with an inlet and an outlet. The cross-β structures are cleared by binding to a cross-β structure-binding compound as defined hereinbefore. It is very important that no elements, such as the cross-β structure-binding compounds from the system, are brought into the subject's circulation. For that reason, among others, preferred systems are dialysis systems.

The invention further provides devices for carrying out methods as disclosed above. Thus, the invention provides a separation device for carrying out a method according to the invention, whereby the apparatus comprises a system for transporting circulation fluids ex vivo, the system provided with means for connection to a suubject's circulation for entry into the system and return from the system to the subject's circulation, the system comprising a solid phase, the solid phase comprising at least one compound capable of binding cross-β structures. Compounds for binding cross-β structures have been disclosed herein. The preferred device is a dialysis apparatus.

The invention also provides for detection of cross-β structures in samples. Such samples may be tissue samples, biopsies and the like, or body fluid samples, such as blood, serum, liquor, cerebrospinal fluid, urine, and the like. The invention thus provides a method for detecting cross-β structures in a sample, comprising contacting the sample with a compound capable of binding cross-β structures, allowing for binding of cross-β structures to the compound and detecting the complex formed through binding.

Cross-β structure-binding compounds have been defined hereinbefore. Detection of the complex or one of its constituents can be done through any conventional means involving antibodies or other specific binding compounds, such as cross-β structure-binding compounds, etc. Detection can be direct, by labeling the complex or a binding partner for the complex or its constituents, or even by measuring a change in a physical or chemical parameter of the complex versus unbound material. It may also be indirect by further binding compounds provided with a label. A label may be a radioactive label, an enzyme, a fluorescent molecule, etc.

The invention further provides devices for carrying out the diagnostic methods. Thus, the invention provides a diagnostic device for carrying out a method according to the invention, comprising a sample container, a means for contacting the sample with a cross-β structure-binding compound, a cross-β structure-binding compound and a means for detecting bound cross-β structures. Preferably, the device comprises a means for separating unbound cross-β structures from bound cross-β structures. This can be typically done by providing the cross-β structure-binding compounds on a solid phase.

### DESCRIPTION OF THE FIGURES

FIG. 1. Schematic representation of the “cross-β structure pathway.” The cross-β structure is found in a number of proteins.⁽¹⁾ The formation of a cross-β structure can be triggered by several physiological or pathological conditions and subsequently initiates a cascade of events, the “cross-β structure pathway.” Among the factors that trigger or regulate the formation of a cross-β structure within a given protein are: 1) the physicochemical properties of the protein, 2) proteolysis, 3) regulated post-translational modification, including cross-linking, oxidation, phosphorylation, glycosylation and glycation, 4) glucose, and 5) zinc. Certain mutations within the sequence of a protein are known to increase the ability of the protein to adopt a cross-β structure and form amyloid fibrils. These mutations are often found in hereditary forms of amyloidosis, for example, in Aβ. The present invention discloses multiple novel examples of proteins capable of adopting a cross-β structure. Several proteins are known to bind cross-β-containing proteins.⁽²⁾ These proteins are part of the herein-disclosed signaling cascade (“cross-β structure pathway”) that is triggered upon formation of a cross-β structure. The “cross-β structure pathway” is modulated in many ways.^{(3,4,5)} Factors that regulate the pathway include modulators of synthesis and secretion, including NO regulators, as well as modulators of activity, including protease inhibitors. The pathway is involved in many physiological and pathological processes including, but not limited to, atherosclerosis, diabetes, amyloidosis, bleeding, inflammation, multiple sclerosis, Parkinson's disease, sepsis, and hemolytic uremic syndrome.⁽⁷⁾ Given the established role for tPA in long-term potentiation, the “cross-β structure pathway” may also be involved in learning.

FIG. 2. Cross-β structure in fibrin. Panel A: Thioflavin T fluorescence of a fibrin clot. A fibrin clot was formed in the presence of Thioflavin T and fluorescence was recorded at indicated time points. Background fluorescence of buffer, Thioflavin T and a clot formed in the absence of Thioflavin T, was subtracted. Panel B: Circular dichroism analysis of fibrin-derived peptides 85, 86 and 87. Ellipticity (Dg.cm²/dmol) is plotted against wavelength (nm). The CD spectra demonstrate that peptides 85 and 86, but not peptide 87, contain β-sheets. Panel C: X-ray fiber diffraction analysis of peptide 85 reveals that the peptide forms cross-β sheets. Panel D: Plasminogen activation assay with fibrin peptides 85, 86 and 87. It is seen that peptides 85 and 86, both containing a cross-β structure, do stimulate the formation of plasmin by tPA, whereas peptide 87, which lacks a cross-β structure, does not.

FIG. 3. Binding of tPA, plasminogen and plasmin to Aβ. Aβ was coated onto plastic 96-well plates. Increasing concentrations of either tPA (Panel A) or plasmin(ogen) (Panel B) were allowed to bind to the immobilized peptide. After extensive washing, tPA and plasmin(ogen) binding was assessed by enzyme-linked immunosorbent assays using anti-tPA and anti-plasminogen antibodies. Binding of tPA (Panel C) and plasmin (Panel D) to Aβ in the presence of 50 mM ε-aminocaproic acid (M-ACA) was assessed as in Panels A and B.

FIG. 4. Stimulation of tPA-mediated plasmin formation by Aβ and synergistic stimulation of cell detachment by plasminogen and Aβ. Panel A: Plasminogen (200 μg/ml) and tPA (200 pM) were incubated with Aβ (5 μM) or control buffer. Samples were taken from the reaction mixture at the indicated periods of time and plasmin activity was measured by conversion of the chromogenic plasmin substrate S-2251 at 405 nm. Panel B: N1E-115 cells were differentiated and received the indicated concentrations of plasmin in the presence or absence of 25 μM Aβ. After 48 hours, the dead cells were washed away and the remaining adherent cells were stained with methylene blue. Plasmin cannot prevent Aβ-induced cell detachment. Panel C: N1E-115 cells were differentiated and received the indicated concentrations of plasminogen in the presence or absence of 10 μM Aβ. After 24 hours, cell detachment was then assessed. Aβ or plasminogen alone do not affect cell adhesion, but cause massive cell detachment when added together. Panel D: Immunoblot analysis of plasmin formation and laminin degradation. Differentiated N1E-115 cells were treated with or without Aβ (10 μM) in the absence or presence of added plasminogen. Addition of Aβ results in the formation of plasmin (bottom panel) and in degradation of laminin (top panel).

FIG. 5. Carboxypeptidase B inhibits Aβ-stimulated tPA-mediated plasmin formation and cell detachment. Panel A: Plasminogen (200 μg/ml) and tPA (200 pM) were incubated with Aβ (5 μM) or control buffer. Samples were taken from the reaction mixture at the indicated periods of time and plasmin activity was measured by conversion of the chromogenic plasmin substrate S-2251 at 405 nm. The reaction was performed in the absence or the presence of 50 μg ml⁻¹ carboxypeptidase B (CpB) and in the absence or presence of 3.5 μM carboxypeptidase inhibitor (CPI). CpB greatly attenuates A-stimulated plasmin formation. Panel B: N1E-115 cells were differentiated and treated with Aβ (10 μM), plasminogen (Plg, 20 μg ml⁻¹) and/or CpB (1 μM) as indicated. After 24 hours, the cells were photographed. Panel C: Subsequently, the cells were washed once with PBS and the remaining cells were quantified as percentage-adhered cells by methylene blue staining. Panel D: Cells were treated as in Panels B and C and medium and cell fractions were collected and analyzed by Western blot using an anti-plasmin(ogen) antibody. Aβ stimulates plasmin formation that is inhibited by CpB.

FIG. 6. Endostatin can form fibrils comprising cross-β structure and stimulates plasminogen activation. Panel A: TEM shows the formation of endostatin fibrils. Panel B: X-ray analysis reveals the presence of cross-β structure in precipitated (prec.) endostatin. Panel C: Plasminogen activation assay demonstrating the stimulating activity of cross-β structure-containing endostatin on tPA-mediated plasmin formation. Aβ is shown for comparison. Panel D: Analysis of endostatin-induced cell death by methylene blue staining. It is seen that only the precipitated form is capable of efficiently inducing cell death. Direct cell death, but not cell detachment, is protected in the presence of sufficient glucose. Buffer prec. indicates control buffer.

FIG. 7. IAPP stimulates tPA-mediated plasminogen activation. Both full-length (fl-hIAPP) and truncated amyloid core (Δ-hIAPP), but not mouse IAPP (Δ-mIAPP), stimulate tPA-mediated plasminogen activation.

FIG. 8. Glycated albumin: Thioflavin T and tPA binding, TEM images, X-ray fiber diffraction. Panel A: ELISA showing binding of tPA to albumin-g6p. Panel B: Competition of tPA binding to albumin-g6p by Congo red as determined using ELISA. Panel C: Fluorescence measurements of Thioflavin T binding to albumin-g6p, which is two, four, or 23 weeks incubated. Panel D: Inhibition of the fluorescent signal obtained upon incubation of 430 nM of albumin-g6p with 19 μM of Thioflavin T by tPA. Panels E and F: Spectrophotometric analysis at 420 nm shows that increasing amounts of tPA results in a decrease of the specific absorbance obtained upon incubation of 500 nM of albumin-g6p with 10 μM of Thioflavin T. Electron micrographs showing amorphous precipitates of four weeks glycated albumin-g6p (Panel G), bundles of fibrillar aggregates of 23 weeks incubated albumin-g6p (Panel H), and two weeks glycated albumin-g6p (Panel I). Panel J: X-ray scattering of albumin-g6p (23 weeks). Scattering intensities are color coded on a linear scale and decreases in the order white-grey-black. Scattering from amorphous control albumin is subtracted, as well as scattering from the capillary glass wall and from air; d-spacings and the direction of the fiber axis are given, and preferred orientations are indicated with arrows. Panel K: Radial scans of albumin control and albumin-g6p (23 weeks). Panel L: Radial scan of albumin-g6p (23 weeks), showing repeats originating from fibrous structure, after subtracting background scattering of amorphous precipitated albumin. d-spacings (in A) are depicted above the peaks. Panel M: Tangential scans along the 2θ scattering-angles, corresponding to indicated d-spacings. The scans show that the 4.7 Å repeat, which corresponds to the hydrogen-bond distance within individual β-sheets, and the 6 Å repeat, are oriented perpendicular to the 2.3 Å repeat that runs parallel to the fiber axis. Panel N: Schematic drawing of the orientation of the cross-β structures in albumin-g6p (23 weeks) amyloid fibrils.

FIG. 9. Fibril formation of human hemoglobin. Panel A: Binding of tPA to in vitro glycated Hb-g6p. Panel B: Electron micrographs showing in vitro glycated Hb, which aggregates in an amorphous and fibrous manner.

FIG. 10. Amyloid properties of albumin-AGE are introduced irrespective of the carbohydrate or carbohydrate derivative used for glycation. Panels A-I: Congo red fluorescence of air-dried albumin preparations. Fluorescence was measured with albumin incubated with buffer (Panel A) or with buffer and NaCNBH₃ (Panel B), with amyloid core peptide of human IAPP (Panel C), Aβ (Panel D), with albumin incubated with g6p (Panel E), glucose (Panel F), fructose (Panel G), glyceraldehyde (Panel H), and glyoxylic acid (Panel I). Panel J: Thioflavin T-amyloid fluorescence was measured in solution with the indicated albumin preparations. Panels K and L: Binding of amyloid-binding serine protease tPA to albumin preparations was assayed using an ELISA set-up. In Panel K, binding of tPA to albumin-glucose, -fructose, -glyceraldehyde, -glyoxylic acid, and albumin-buffer controls is shown. In Panel L, binding of tPA to positive controls albumin-g6p, Aβ and IAPP is shown, as well as to albumin incubated with control buffer.

FIG. 11. Analysis of Congo red and tPA binding to Aβ. Panel A: Binding of tPA to immobilized Aβ, as measured using an ELISA. Panel B: Influence of increasing concentrations of Congo red on binding of tPA to Aβ. In the ELISA, 10 μg ml⁻¹ of Aβ(1-40) was coated and incubated with 40 nM of tPA and 0-100 μM of Congo red.

FIG. 12. Binding of human FXII to amyloid peptides and proteins that contain the cross-β structure fold. Panels A and B: Binding of FXII to prototype amyloid peptides hAβ(1-40) and human fibrin fragment α₁₄₇₋₁₅₉ FP13, and albumin-AGE and Hb-AGE, which all contain cross-β structure, were tested in an ELISA. FXII does not bind to negative controls mouse Δ islet amyloid polypeptide (ΔmIAPP), albumin-control and Hb-control: all three lack the amyloid-specific structure. k_{D}s for hAβ(1-40), FP13, albumin-AGE and Hb-AGE are approximately 2, 11, 8 and 0.5 nM, respectively. Panels C and D: Coated hAβ(1-40) was incubated with 2.5 nM FXII in binding buffer, in the presence of a concentration series of human recombinant tissue-type plasminogen activator (Actilyse®, full-length tPA), or Reteplase® (K2P-tPA). The full-length tPA and K2P-tPA concentration was, at maximum, 135 times the k_{D} for tPA binding to hAβ(1-40) (50 nM). Panels E and F: Coated amyloid albumin-AGE was incubated with 15 nM FXII in binding buffer, in the presence of a concentration series of full-length tPA or K2P-tPA. The tPA concentration was, at maximum, 150 times the k_{D} for tPA binding to albumin-AGE (1 nM). Panel G: Binding of FXII to hAβ(1-40) and the prototype amyloid human amylin fragment hΔIAPP was tested using dot blot analysis. Ten μg of the peptides that contain cross-β structure, as well as the negative control peptide mΔIAPP and phosphate-buffered saline (PBS), were spotted in duplicate. FXII specifically bound to hAβ(1-40), as well as to hΔIAPP.

FIG. 13. Finger domains bind to amyloid (poly)peptides. Panel A: Binding of tPA and K2P-tPA to albumin-g6p. Panel B: Binding of tPA and K2P-tPA to Aβ(1-40). The tPA antibody used for detection recognizes both tPA and K2P-tPA with equal affinity (not shown). Panel C: Binding of tPA-F-GST and tPA to immobilized Aβ(1-40) and albumin-g6p. Control RPTPμ-GST does not bind Aβ or albumin-g6p. Panel D: Pull-down assay with insoluble Aβ fibrils and tPA domains. Conditioned BHK medium from stably transfected cell-lines expressing tPA-F, F-EGF, EGF, F-EGF-K1 and K1 with a C-terminal GST tag, as well as the tag alone, was used. “Control,” medium before the pull-down, “Aβ,” washed amyloid Aβ pellet, after the pull-down, “Sup,” medium after extraction with Aβ. Samples were analyzed on Western blot using rabbit anti-GST antibody Z-5. Panels E-G: ELISA showing binding of tPA-F-EGF-GST and full-length recombinant tPA to amyloid Aβ (Panel E), FP13 (Panel F) and IAPP (Panel G). MΔIAPP was coated as non-amyloid negative control (Panel E). Peptides were immobilized on ELISA plates and overlayed with concentration series of tPA and F-EGF-GST. GST was used as a negative control. Binding was detected using rabbit anti-GST antibody Z-5. Panels H-M: Immunohistochemical analysis of binding of tPA-F-EGF-GST to amyloid deposits in human brain afflicted with AD. Brain sections were overlayed with tPA-F-EGF-GST (Panels H and J) or negative control GST (Panel L). The same sections were incubated with Congo red (Panels I, K, and M) to locate amyloid deposits. Panels N and O: Pull-down assay with insoluble Aβ fibrils and finger domains. Recombinant F domains with a C-terminal GST tag were expressed by stably transfected BHK cells. “Control,” medium before the pull-down, “Aβ,” washed amyloid Aβ pellet, after the pull-down, “Sup,” medium after extraction with Aβ. Samples were analyzed on Western blot using rabbit anti-GST antibody Z-5.

FIG. 14. The finger module. Panel A: Schematic representation of the location of the finger domain in tPA, factor XII, HGFa and fibronectin. Panel B: Alignment of the amino acid sequence of the finger domain of the respective proteins. Panel C: Representation of the peptide backbone of the tPA finger domain and the fourth and fifth finger domain of FN. Conserved disulfide bonds are shown in ball and stick. (SEQ ID NOS:36-50.)

FIG. 15. Antibodies elicited against amyloid peptides cross-react with glycated proteins, and vice versa. Panels A-C: ELISA with immobilized g6p-glycated albumin-AGE:23 and Hb-AGE, their non-glycated controls (Panel A), Aβ(1-40) (Panel B), and IAPP and mΔIAPP (Panel C). For the Aβ ELISA, polyclonal anti-human vitronectin antibody α-hVn K9234 was used as a negative control. Panel D: Binding of α-AGE1 to immobilized Aβ(1-40) on an ELISA plate, after pre-incubation of α-AGE1 with IAPP fibrils. Panel E: Pull-down assay with anti-AGE1 antibody and amyloid fibrils of Aβ(16-22) (lanes 1-2), Aβ(1-40) (lanes 4-5) and IAPP (lanes 6-7). After pelleting and washing of the fibrils, samples were boiled in non-reducing sample buffer and analyzed by SDS-PAGE. s=supernatant after amyloid extraction, p=amyloid pellet after extraction, M=molecular marker. Panels F and G: In an ELISA set-up, immobilized Aβ(1-40) (Panel F) and IAPP (Panel G) are co-incubated with tPA and 250 or 18 nM α-AGE1, respectively. Panel H: In an ELISA set-up, binding of α-Aβ(1-42) H-43 to immobilized positive control Aβ(1-40), and to IAPP and albumin-AGE:23 is tested. Albumin-control:23 and MΔIAPP are used as negative controls. Panel 1: Binding of 100 nM α-Aβ(1-42) H-43 to IAPP, immobilized on an ELISA plate, in the presence of a concentration series of tPA. Panels J and K: ELISA showing binding of a polyclonal antibody in mouse serum elicited against albumin-AGE:23 and Aβ(1-40) (ratio 9:1) (“poab anti-amyloid”) and of a polyclonal antibody elicited against a control protein (“control serum”) to immobilized IAPP (Panel J) and albumin-AGE:23 (Panel K). Serum was diluted in PBS with 0.1% v/v TWEEN20®. Panel L: ELISA showing binding of mouse poab anti-amyloid serum to amyloid Aβ(1-40), hΔIAPP and fibrin fragment α₁₄₈₋₁₆₀ FP13. Control serum with antibodies raised against an unrelated protein, buffer and immobilized non-amyloid mΔIAPP and fibrin fragment α₁₄₈₋₁₅₇ FP10 were used as negative controls. Panel M: Immunohistochemical analysis of the binding of rabbit anti-AGE2 to a spherical amyloid plaque (arrow) in a section of a human brain afflicted with Aβ. Magnification 400×. Panel N: Congo red fluorescence of the same section. Magnification 630×.

FIG. 16. Monoclonal anti-cross-β structure antibody 3H7 detects glycated hemoglobin, Aβ, IAPP and FP13. ELISA showing binding of mouse monoclonal anti-cross-β structure antibody 3H7 to glycated hemoglobin vs. control unglycated hemoglobin (Panel A) or Aβ, hIAPP, ΔmIAPP and fibrin fragment α₁₄₈₋₁₆₀ FP13 (Panel B).

FIG. 17. Sandwich ELISA for detection of amyloid albumin-AGE or amyloid hemoglobin in solution. Immobilized recombinant tPA on Exiqon protein Immobilizers was overlayed with albumin-AGE:23 solution or albumin-control:23 solution at the indicated concentrations. Next, bound amyloid structures were detected with anti-Aβ(1-42) H-43 (A).

FIG. 18. Binding of tPA, factor XII, fibronectin and finger domains thereof to compounds with cross-β structure. Panels A-C: Full-length purified tPA (Panel A), factor XII (Panel B) and fibronectin (Panel C) bind to immobilized peptides with cross-β structure conformation in an ELISA. Panels D-F: In an ELISA, the recombinant Fibronectin type I, or finger domains (Panel F) of tPA (Panel D), factor XII (Panel E) and fibronectin (Panel F) bind specifically to immobilized amyloid-like peptides with cross-β structure conformation. The control-free GST tag does not bind. Panel G: In an ELISA, recombinantly expressed fibronectin type I domains 4-5 of fibronectin, N-terminally tagged to growth hormone and a His₆-tag, and C-terminally tagged to a His₆-tag (FnF4,5his), specifically captures Hb-AGE from solution, and not control hemoglobin. Panels H and I: Activation of the contact system and the fibrinolytic system in patients with systemic amyloidosis. Panel H: Plasma samples of 40 apparently healthy controls (19 male, 21 female, average age 49.4 (stand. dev. 6.8 years)) and of 40 patients with systemic amyloidosis (17 male, 23 female, average age 51.8 (stand. dev. 9.9 years)) were tested for plasmin-α2-anti-plasmin (PAP) levels with an ELISA. One patient revealed a PAP level of 88.3 μg ml⁻¹, which is not shown in the graph for clarity. Panel I: In the same plasma samples, levels of FXIIa were measured with an ELISA. Sixteen out of 40 patients with systemic amyloidosis have elevated levels of FXIIa.

FIG. 19. Activation of factor XII by kaolin and by peptide aggregates with cross-β structure conformation. Panel A: Like kaolin, amyloid-like peptide aggregates of FP13 and Aβ stimulate the activation of factor XII, as detected by the conversion of Chromozym PK, upon formation of kallikrein from prekallikrein by activated factor XII. Buffer control and non-amyloid controls FP10 and mIAPP do not activate factor XII. Panel B: Like FP13 and Aβ, also cross-β structure-rich peptides LAM12 and TTR11 stimulate factor XII activation, to a similar extent as kaolin. Panel C: In the chromogenic factor XII/kallikrein activity assay, the stimulatory activity of 150 μg/ml kaolin is strongly dependent on the presence of 1 mg/ml albumin in the assay buffer. Albumin alone also shows to some extent factor XII/prekallikrein activating properties. Panel D: Contacting plasma, lysozyme and γ-globulins to DXS500k results in activation of tPA and plasminogen, as measured in the chromogenic tPA/Plg activation assay. DXS500k alone also results in some activation. Plasma, lysozyme or γ-globulins controls do not activate tPA and Plg. Panel E: Overnight incubation at room temperature of plasma with kaolin or DXS500k results in increased fluorescence of amyloid dye Thioflavin T, when compared to incubation with buffer. Panel F: Incubation of γ-globulins with kaolin or DXS500k also induces increased ThT fluorescence. Panel G: Only DXS500k induces ThT fluorescence with lysozyme. Kaolin incubation results only in a small increase in ThT fluorescence, when compared to buffer. Panels H-K: In an ELISA set-up, tPA binds specifically to plasma proteins (Panel H), γ-globulins (Panel I), lysozyme (Panel J) and factor XII (Panel K) that were pre-incubated overnight with DXS500k, whereas tPA does not bind to buffer-incubated proteins. K2P-tPA that lacks the F domain does not bind to surface-contacted proteins. Panel L: In the tPA ELISA, glycated hemoglobin (Hb-AGE) with amyloid-like properties was used as a positive control for tPA binding. Panel M: Auto-activation of factor XII is established by incubating purified factor XII with DXS500k or with various amyloid-like protein aggregates with cross-β structure conformation, in the presence of chromogenic substrate S-2222.

FIG. 20. Proteins and peptides with amyloid cross-β structure activate blood platelets and can induce platelet aggregation. Panels A-D: Combined freshly isolated platelets from three healthy human donors are incubated with concentration series of proteins and peptides with cross-β structure conformation, and with controls. Activation of the platelets is analyzed on Western blots by measuring phosphorylation of p38MAPK after one minute and five minutes of peptide/protein incubation. Concentrations used were 6.25, 25 and 100 μg/ml for hemoglobin, and 5, 25 and 125 μg/ml for the other peptides/proteins used. Panels A and B: Incubation of platelets with amyloid hemoglobin-AGE results in platelet activation similar to the positive control native low-density lipoprotein after one minute (Panel A). After five minutes, Hb-AGE shows a prolonged activation, whereas p38MAPK is no longer phosphorylated by nLDL stimulation (Panel B). Incubation with control hemoglobin results in background levels of p38MAPK phosphorylation, similar to buffer. Panels C and D: Amyloid peptides FP13 and Aβ already potently induce p38MAPK phosphorylation after one minute incubation (Panel C), whereas amyloid denatured γ-globulins and transthyretin amyloid fragment TTR11 induce p38MAPK phosphorylation only after five minutes stimulation (Panel D). Panel E: In a blood platelet aggregometer, the influence of amyloid FP13 and denatured γ-globulins is tested and compared to the effect of thrombin on aggregation. Negative controls were HEPES Tyrode buffer and 200 μg/ml native γ-globulins. Both FP13 and denatured γ-globulin induce platelet aggregation in a dose-dependent manner. Panel F: Storage of fresh platelets for 72 hours at room temperature results in increased Thioflavin T fluorescence.

FIG. 21. Amyloid-like conformations are presented on activated blood platelets and contribute to platelet aggregation. Panels A and B: Analysis of amyloid formation during adhesion of platelets in whole blood to vWF (Panel A) or collagen (Panel B) under flow for five minutes. Samples were stained with the amyloid-specific dye Congo Red. Panels C and D: FACS analysis of platelets stimulated with (Panel D) or without (Panel C) thrombin (one minute, 37° C.) in the presence of EDTA. The fluorescent amyloid dye Thioflavin T was used to detect amyloid on the surface of platelets. Panel E: Washed platelets were exposed to thrombin-activating peptide (TRAP) in the presence or absence of ThT (200 μM), Congo Red (200 μM) or tPA (1 μM) where indicated. Platelet aggregation was assessed by light scattering. Panel F: Activation of platelets in the presence of TRAP, indo and AR, with or without tPA. Indo: indomethacin (aspirin-like), AR-C6993MX (clopidogrel-like). TPA further decreases the level of TRAP-induced platelet activation that is suppressed by indo and AR.

FIG. 22: Binding of factor XII and tPA to β2-glycoprotein I and binding of anti-β2gpi auto-antibodies to recombinant β2gpi. Panel A: Chromogenic plasmin assay showing the stimulatory activity of recombinant β2gpi on the tPA-mediated conversion of plasminogen to plasmin. The positive control was amyloid fibrin peptide FP13. Panel B: In an ELISA, recombinant β2gpi binds to immobilized tPA, whereas β2gpi purified from plasma does not bind. The k_{D} is 2.3 μg/ml (51 nM). Panel C: In an ELISA, factor XII binds to purified recombinant human β2gpi, and not to β2gpi that is purified from human plasma, when purified factor XII is immobilized onto ELISA plate wells. Recombinant β2gpi binds with a k_{D} of 0.9 μg/ml (20 nM) to immobilized factor XII. Panel D: Western blot incubated with anti-human factor XII antibody. The β2gpi was purified either from fresh human plasma or from plasma that was frozen at −20° C. and subsequently thawed before purification. When comparing lanes 2-3 with 4-5, it is shown that freezing-thawing of plasma results in co-purification of factor XII, together with the β2gpi. The molecular mass of factor XII is 80 kDa. Panel E: In an ELISA, recombinant β2gpi efficiently inhibits binding of anti-β2gpi auto-antibodies to immobilized β2gpi, whereas plasma β2gpi has a minor effect on antibody binding. Anti-β2gpi auto-antibodies were purified from plasma of patients with the auto-immune disease Anti-phospholipid syndrome. Panel F: Exposure of 25 μg/ml β2gpi, recombinantly produced (rβ2gpi) or purified from plasma (nβ2gpi), to 100 μM cardiolipin vesicles or to 250 μg/ml dextransulphate 500,000 Da (DXS) induces an increased fluorescence of Thioflavin T, suggestive for an increase in the amount of cross-β structure in solution. Signals are corrected for background fluorescence of cardiolipin, DXS, ThT and buffer. Panel G: Recombinant β2gpi binds to a higher extent to tPA, which is immobilized on the wells of an ELISA plate, than β2gpi purified from human plasma. Panel H: Binding of tPA and K2P-tPA to β2gpi immobilized on the wells of an ELISA plate, or to β2gpi bound to immobilized cardiolipin is assessed. B2gpi contacted to cardiolipin binds tPA to a higher extent than β2gpi contacted to the ELISA plate directly. K2P-tPA does not bind to β2gpi. TPA does not bind to immobilized cardiolipin. Panel I: Recombinant β2gpi induces platelet activation as assayed by measuring the extent of platelet p38MAPK phosphorylation. In contrast, β2gpi purified from human plasma, induced p38MAPK phosphorylation to a lesser extent.

FIG. 23. Amyloid-like properties of oxidized low-density lipoprotein. Panel A: In time, an increase of the oxidation of LDL, as measured by specific diene fluorescence at 243 nm, is accompanied by an increase in Thioflavin T fluorescence and a decrease in Congo red fluorescence, indicative for structural changes in the apoB protein part of the LDL. Panel B: Congo red fluorescence of 25 μg/ml oxidized LDL is similar to the Congo red fluorescence of the positive control, 25 μg/ml Aβ. Native LDL also shows Congo red fluorescence to some extent. Panel C: In the chromogenic plasmin assay, 24% oxidized LDL shows cofactor activity for the tPA-mediated conversion of plasminogen to plasmin, whereas native, LDL has hardly any effect on tPA activity. Panel D: Factor XII in plasma is activated by oxidized LDL and by amyloid peptide FP13 K157G, as determined with a chromogenic factor XII activation assay using chromogenic substrate S-2222.

FIG. 24. A fibrin clot comprises amyloid-like cross-β structure conformation. Panel A: Fibrin clots show fluorescent signals when stained with amyloid-specific dyes Congo red, Thioflavin S and Thioflavin T. As a control, images of fibrin clots as seen under direct light and under the FITC and TRITC excitation wavelengths are shown. Panel B: Fibrin clots stain positive with Congo red (CR), Thioflavin S (ThS) and Thioflavin T (ThT), indicative for the presence of amyloid-like cross-β structure aggregates. Panels C-E: In an aPTT coagulation test, coagulation of human plasma is delayed in the presence of amyloid-specific dyes Congo red (Panel C), ThS (Panel D) and ThT (Panel E), whereas buffer controls do not influence coagulation (Na₂SO₄ for CR and NH₄Cl for ThT/ThS, respectively). These observations are indicative for a role of amyloid-like cross-β structure conformation in the formation of a fibrin polymer network. Panels F-H: In PT coagulation tests, similar inhibitory activities of amyloid-specific dyes CR (Panel F), ThS (Panel G), and ThT (Panel H) on fibrin clot formation are observed as in aPTT.

FIG. 25. Overview of the domain structure of tPA, Factor XII and Fibronectin, all recombinant constructs made thereof and primers used for preparing the recombinant constructs. Primer (1) (SEQ ID NO:10); Primer (2) (SEQ ID NO:11); Primer (3) (SEQ ID NO: 16); Primer (4) (SEQ ID NO:18); Primer (5) (SEQ ID NO:20); Primer (6) (SEQ ID NO:23); Primer (7) (SEQ ID NO:24); Primer (8) (SEQ ID NO:52); Primer (9) (SEQ ED NO:53); Primer (10) (SEQ ID NO:54); Primer (18) (SEQ ID NO:55); Primer (12) (SEQ ID NO:56); Primer (13) (SEQ ID NO:57).

FIG. 26. Reactive oxygen species production by microvascular bEnd.3 endothelial cells upon exposure to Escherichia coli TOP10 cells is reversed by pre-incubation of the pathogen with crossbeta structure-binding compounds. FIG. 26A: Binding of crossbeta structure-binding compound IgIV to immobilized glycated hemoglobin comprising crossbeta structure in an ELISA set-up. FIG. 26B: ROS production by bEnd.3 ECs upon exposure to 1/120^{th} volume PBS, 100 μM H₂O₂, 6×10⁸ E. coli TOP10 cells/ml or 6×10⁸ E. coli cells/ml that were pre-incubated with crossbeta structure-binding compounds Congo red, ThT, tPA and IgIV (“cbs binders”). Up-regulation of ROS by the ECs was determined by measuring fluorescence of a probe for ROS. PBS was used as a negative control; 100 μM H₂O₂ was used as a positive control for ROS induction.

FIG. 27. Activation of factor XII by Escherichia coli strain TOP10 is inhibited by crossbeta structure-binding compounds, and reactive oxygen species production by bEnd.3 endothelial cells is inhibited by crossbeta structure-binding compounds. FIG. 27A: Exposure of cultured murine brain microvascular endothelial cells (bEnd.3) to E. coli Top10 induces up-regulation of reactive oxygen species (ROS), as measured in a kinetic fluorescent assay. Serial pre-incubation of the E. coli cells with crossbeta structure binders Thioflavin T, Congo red and a mixture of tissue-type plasminogen activator and intravenous immunoglobulins (“cbs binders”) lowers ROS levels induced by E. coli. PBS was used as a negative control; 100 μM H₂O₂ was used as a positive control for ROS induction. FIG. 27B: Escherichia coli TOP10 (E. coli) induces factor XII activity in a chromogenic factor XII/prekallikrein activation assay. Serial pre-incubation of the E. coli cells with crossbeta structure binders Thioflavin T, Congo red and a mixture of tissue-type plasminogen activator and intravenous immunoglobulins (“cbs binders”) diminishes factor XII activation by E. coli to background levels observed with buffer only. Kaolin at 150 μg/ml was used as a positive control. Including factor XII in the experiments was essential; discarding factor XII did not result in any conversion of the chromogenic kallikrein substrate (not shown).

FIG. 28. Staphylococcus aureus Newman and Escherichia coli TOP10 induce platelet aggregation, which is inhibited by crossbeta structure-binding compounds. FIG. 28A: Platelets in platelet-rich plasma (PRP) from healthy human donor A readily aggregate upon stimulation by Staphylococcus aureus Newman (S. aureus). Pretreatment of the S. aureus cells with crossbeta structure-binding compounds Thioflavin T, Congo red and a mixture of tPA and IgIV (S. aureus+cbs binders) inhibits S. aureus-induced platelet aggregation. FIG. 28B: Similar experiment as in FIG. 28A. Now, S. aureus cells were used after 24 hours storage at 4° C., and blood was donated by anonymous donor B. Again, S. aureus readily induces platelet aggregation, which is in part reversed upon pre-treatment of S. aureus with crossbeta structure-binding compounds. FIG. 28C: Platelets aggregate when exposed to Escherichia coli TOP10 cells. Crossbeta structure-binding molecules reverse these platelet-activating properties. FIG. 28D: Positive and negative controls TRAP and buffer. The graph shows that TRAP only activates platelets when present at a cut-off level of over 2 μM.

FIG. 29. Binding of tPA to E. coli TOP10 and S. aureus Newman, assessed with a tPA-plasminogen activation assay. Binding of tPA to E. coli TOP10 and S. aureus Newman was determined using a tPA/plasminogen activation assay including a chromogenic plasmin substrate. The bacteria were pre-incubated with buffer (E. coli, S. aureus) or with crossbeta structure-binding compounds (“cbs binders”); first 2.5 mM Thioflavin T, then 5 mM Congo red and finally 25 μM tPA+25 mg/ml IgIV (E. coli+cbs binders, S. aureus+cbs binders). In the assay, 1.3×10⁸ E. coli cells/ml and 1.8×10⁸ S. aureus cells/ml are used. tPA is omitted in the reaction mixture and, therefore, plasmin generation can only occur when an external source of tPA is introduced in the assay.

FIG. 30. Activation of the contact system of coagulation by E. coli MC4100 either exposing (or not exposing) amyloid crossbeta structure-comprising core protein curli. Exposing factor XII, prekallikrein, high-molecular weight kininogen and chromogenic kallikrein substrate Chromozym-PK to E. coli MC4100 grown for 44 hours at 26° C. (+curli) results in more potent activation of the contact system of coagulation than exposure to E. coli MC4100 grown for 24 hours at 37° C. (−curli). Negative control: 1 mg/ml bovine serum albumin; positive control: 1 mg/ml bovine serum albumin+150 μg/ml kaolin.

FIG. 31. Production of ROS by bEnd.3 ECs is influenced by E. coli and S. aureus pre-incubated with crossbeta structure-binding compounds. FIG. 31A: ROS production was followed in time using fluorescent probe CM-H2DCFDA. As positive control, overnight cultured ECs (64×10³ cells/well) were exposed to a concentration series of H₂O₂. FIG. 31B: Influence of ROS production upon exposure of ECs (128×10³ cells/well, cultured overnight) to E. coli TOP10 cells and to bacteria that were pre-incubated with crossbeta structure-binding compounds Thioflavin T, Congo red, tPA, IgIV is shown, as well as of ECs that were co-incubated with E. coli and indicated crossbeta structure-binding compounds. FIG. 31C: As in FIG. 31B, but with S. aureus Newman cells.

FIG. 32. Influence of pathogens on coagulation. Role of crossbeta structure-binding compounds. FIG. 32A: In a PT set-up, coagulation of human plasma was determined after pre-incubating the plasma with buffer or with 6.5×10⁹ E. coli TOP10 cells/ml. The E. coli were pre-incubated with PBS (“buffer”) or with crossbeta structure-binding compounds ThT, Congo red, tPA, IgIV (“cbs binders”). FIG. 32B: In an aPTT set-up, the same samples were analyzed as in FIG. 32A.

FIG. 33. Tissue factor up-regulation in THP-1 monocytes upon stimulation with S. aureus is blocked by crossbeta structure-binding compounds. FIG. 33A: S. aureus induces up-regulation of TF synthesis in THP-1 monocytes, as assessed with a chromogenic activated factor X assay in the presence of substrate S2765 and activated factor VII. Upon pre-incubation of S. aureus with crossbeta structure-binding compounds ThT, Congo red, tPA and IgIV, TF expression returns to basal levels observed with negative control (buffer incubated THP-1). FIG. 33B: Glycated hemoglobin (Hb-AGE) and amyloid-β (Aβ) are potent inducers of TF up-regulation in THP-1 monocytes. Negative controls: buffer, freshly dissolved Aβ, freshly dissolved Hb; positive control: 10 μg/ml LPS.

FIG. 34. Platelet aggregation stimulated by proteins with amyloid cross-β structure conformation. Aggregation is induced by proteins with amyloid cross-β structure conformation: Amyloid-O peptide, advanced glycation end product-modified Hemoglobin-AGE, BSA-AGE, Herpes simplex virus Glycoprotein, B (gB), fibrin α-chain peptide 12 (FP12) and fibrin α-chain peptide 13 (FP13, Kranenburg, 2002). Concentration dependency of crossbeta structure-induced platelet aggregation is compared to the stimulation of platelets by 8 μM TRAP (set to 100%).

FIG. 35. Platelet aggregation by crossbeta structure under influence of various inhibitors of signaling platelet pathways. Aggregation of washed platelets exposed to 50 μg/ml Amyloid β protein (Aβ) was inhibited by 20 ng/ml PGI2 analogue Iloprost (FIG. 35A). 30 μM Indomethacin (Indo), 50 nM AR-C69931MX (AR) or both could partially inhibit amyloid-induced aggregation (FIG. 35A). After adding Indomethacin and/or AR-C69931MX to platelets, Hemoglobin-AGE and BSA-AGE still stimulated the cells to aggregate while freshly prepared control solutions did not (FIG. 35B). Addition of 20 ng/ml Iloprost resulted in complete blockage of aggregation in case of each model protein (FIG. 35C).

FIG. 36. Influence of crossbeta structure-binding molecules on crossbeta structure-induced platelet aggregation. FIG. 36A: TRAP-induced platelet aggregation is not influenced by a concentration series of RAGE. FIG. 36B: RAGE effectively inhibits platelet aggregation induced by glycated hemoglobin. FIG. 36C: Similar to glycated hemoglobin, glycated albumin-induced aggregation is efficiently blocked with sRAGE. FIGS. 36D-36F: Intravenous immunoglobulins (IgIV) effectively prevent glycated albumin-induced platelet aggregation (FIG. 36D) as well as glycated hemoglobin-induced aggregation (FIG. 36E), whereas TRAP-induced platelet aggregation is barely influenced by IgIV (FIG. 36F). FIGS. 36G and 36H: tPA efficiently prevents amyloid-β-induced aggregation (FIG. 36G) or glycated hemoglobin-induced aggregation (FIG. 36H). tPA does not influence TRAP-induced aggregation (FIG. 36I).

FIG. 37. Influence of crossbeta structure-binding compounds RAGE and fibronectin type I domain on coagulation. By performing aPTT analyses (FIGS. 37A, 37C, and 37E) and PT analyses (FIGS. 37B, 37D, and 37F) in the presence of concentration series of sRAGE (FIGS. 37A and 37B), HGFA-F (FIGS. 37C and 37D) and tPA-F (FIGS. 37E and 37F), the influence of crossbeta structure-binding compounds on fibrin clot formation was tested. FIG. 37F: fibronectin type. I domain or finger domain.

FIG. 38. Fibrin clot lysis under influence of tPA, truncated tPA and tPA in combination with crossbeta structure-binding compound sRAGE. Fibrin clot lysis was followed in time during incubation of a clot at 37° C. with 1 μM tPA or K2P-tPA. Crossbeta structure-binding compound sRAGE at 67 or 128 μg/ml was added to tPA-incubated clots. The mean absorbance of six independent measurements was calculated. Lysis by tPA was set arbitrarily to 100%.

FIG. 39. Binding of human Anti-phospholipid Syndrome patient auto-antibodies to auto-antigen β2-glycoprotein I is reduced by crossbeta structure-binding compound tPA. Purified auto-antibodies against β2gpi, isolated from plasma of patients suffering from autoimmune disease Anti-Phospholipid Syndrome (APS), bind to immobilized β2gpi autoantigen, which is denatured at the surface of a high-absorbing ELISA plate. Binding of the auto-antibodies is strongly diminished by tPA, a crossbeta structure-binding compound.

FIG. 40. Influence of crossbeta structure-binding compounds IgIV and HGFA-F on bleeding time in an in vivo mouse bleeding time assay. FIG. 40A: In a mouse tail cut assay, both HGFA-F (approximately 234 μg/ml final concentration) and IgIV (approximately 2.5 mg/ml final concentration) prolong bleeding time significantly. Buffer (PBS) was used as a reference for bleeding time. Ten IE heparin per-mouse was used in a positive control group of prolonged bleeding time. Calculates means and error bars are given. FIG. 40B: The averaged data as shown in FIG. 40A, are now displayed in a scatter plot in order to provide insight in the distribution of measured bleeding times. Note: bleeding times exceeding 20 minutes were set to 20 minutes and bleeding was stopped and, in addition, excessive bleeding resulting in blood loss of over 200 μl was also set to a bleeding time of 20 minutes and bleeding was stopped.

FIG. 41. Schematic representation of the blood coagulation cascade: induction of coagulation and fibrinolysis by proteins comprising crossbeta structure, and thrombosis and/or bleeding during pathological conditions. Crossbeta structures act on the hemostatic system and fibrinolytic pathway in various ways. Sources of crossbeta structure that trigger the systems are, for example, misfolded proteins, pathogens with amyloid core proteins, fibrin, and apoptotic or necrotic cells. Crossbeta structures are induced by, for example, stress conditions like, for example, heat, aberrant pH, oxidative stress, excessive glycation, or by exposure of proteins to self or non-self denaturing surfaces like, for example, negatively charged lipids like, for example, phosphatidylserine and cardiolipin, or lipopolysaccharides. (1) Crossbeta structure-comprising proteins bind and activate coagulation factor XII (present in blood leading to activation of the intrinsic pathway of coagulation). (2). Crossbeta structure-comprising proteins activate blood platelets, contributing to their aggregation. (3) Crossbeta structure-comprising proteins stimulate nucleated cells, such as, for instance, endothelial cells and macrophages to induce expression and/or release and/or activation of tissue factor. Tissue factor induces coagulation via the extrinsic pathway. Both the intrinsic and extrinsic pathways of coagulation result in formation of a fibrin clot, which, in itself, is a new source of crossbeta structure that further interacts with the various activation routes of the hemostatic system (“positive feedback”). (4) Factor XII activation also results in production of vaso-active bradykinin, which is in turn involved in activation of the release of intracellular tPA that will result in fibrinolytic activity. Activation of tPA is triggered by crossbeta structure and, amongst other activators, especially by fibrin-comprising crossbeta structure. (5) Stimulation and damage of nucleated cells and/or tissue by crossbeta structure facilitates the release of intracellularly stored tPA, resulting in clot-dissolving fibrinolytic activity, thereby removing fibrin-comprising crossbeta structure. (6) Moreover, stress factors like, for example, crossbeta structure, heat, oxidative stress, changes in pH and/or changes in shear rate are deleterious to cells and tissue by amongst other mechanisms inducing the generation of reactive oxygen species (“ROS”) that play a major role in interfering with the athero-protective endothelium-dependent nitric oxide, system (a), thereby inducing apoptosis/necrosis, accompanied by crossbeta structure formation (b). In this indirect way, new crossbeta structure-rich proteins originating from cells are formed that further contribute to a pro-coagulant state and/or pro-fibrinolytic state via any of the described mechanisms. Over-activation of coagulation and platelet aggregation pathways, as well as hypo-fibrinolytic activity, lead to a pro-coagulant state and is a profound risk factor for thrombosis. In an opposite manner, hyper-fibrinolysis and reduced coaguability of the blood and/or impaired platelet activation, result in bleeding disorders. In conclusion, it is possible to use therapeutic interference in the depicted pathways that are triggered or mediated by crossbeta structure compounds at every stage of the pathways in which crossbeta structure plays a role.

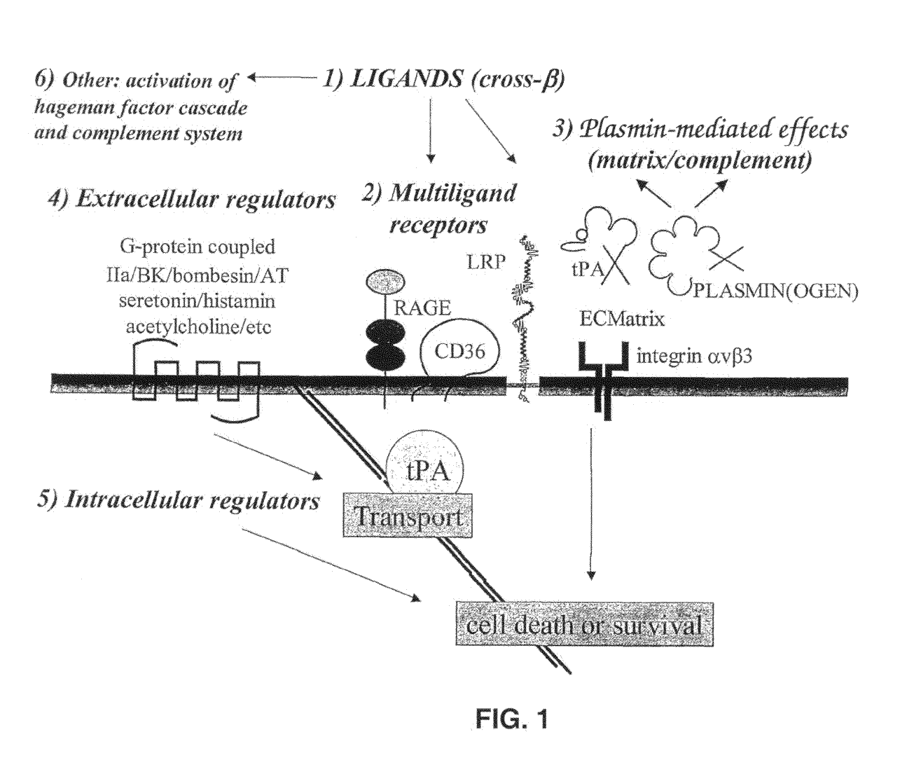

### DETAILED DESCRIPTION OF THE INVENTION

The invention discloses, amongst other biological aspects, (i) the identification of a “cross-β structure pathway” (also called “cross-β pathway”), (ii) the identification of multiligand receptors as being cross-β structure receptors, (iii) the identification of the finger domain as a cross-β structure-binding module, (iv) the identification of finger-containing proteins, including tPA, FXII, HGFA and fibronectin as part of the “cross-β structure pathway,” and (v) means and methods to modulate blood coagulation and fibrinolysis.

This invention further provides compounds not previously known to bind cross-β structure.

As disclosed herein, the invention provides compounds and methods for the detection and treatment of diseases associated with the excessive formation of cross-β structure. Such diseases include known conformational diseases, including Alzheimer disease and other types of amyloidosis. In addition, the present invention also discloses that other diseases not yet known to be associated with excessive formation of cross-β structure are also caused by excessive formation of cross-β structure. Such diseases include atherosclerosis, sepsis, disseminated intravascular coagulation, hemolytic uremic syndrome, preeclampsia, rheumatoid arthritis, autoimmune diseases, thrombosis, bleeding and cancer.

According to the invention, a cross-β structure-binding molecule preferably comprises a finger domain or a molecule containing one or more finger domains, or a peptidomimetic analog of one or more finger domains. The compound can also be an antibody or a functional fragment thereof directed to the cross-β structure, as well as IgIV and/or at least one chaperone.

According to the invention, the compound may also be a multiligand receptor or fragment thereof. The compound may, e.g., be RAGE, CD36, Low-density lipoprotein-Related Protein (LRP), Scavenger Receptor B-1 (SR-BI), SR-A or a fragment of one of these proteins.

The finger domains, finger-containing molecules or antibodies may be human, mouse, rat or from any other species.

According to the invention, amino acids of the respective proteins may be replaced by other amino acids that may increase/decrease the affinity, the potency, bioavailability and/or half-life of the peptide. Alterations include conventional replacements (acid-acid, bulky-bulky, and the like), introducing D-amino acids, making peptides cyclic, etc.

Furthermore, the invention provides, amongst other things, compounds and methods:
1) for detecting the presence of cross-β structure;
2) for inhibiting the formation of amyloid fibrils, and/or aggregates or deposits of proteinaceous molecules comprising crossbeta structure;
3) for modulating cross-β structure-induced toxicity;
4) for the removal and/or shielding of cross-β structure-containing molecules from the circulation.

This invention provides methods for preparing an assay to measure cross-β structure in sample solutions.

This invention provides methods for detecting cross-β structure in tissue samples or other samples obtained from living cells or animals, preferably a human individual.

This invention provides compounds and methods for preparing a composition for inhibiting cross-β structure fibril formation and formation of any crossbeta structure.

This invention provides compounds and methods for preparing a composition for modulating cross-β structure-induced toxicity.

Abbreviations: Aβ, amyloid-β peptide; AD, Alzheimer's disease; AGE, advanced glycation end products; CpB, carboxypeptidase B; CPI (carboxypeptidase inhibitor); ELISA, enzyme-linked immunosorbent assay; FN, fibronectin; FXII, factor XII (Hageman factor); HGFA, hepatocyte growth factor activator; IAPP, islet amyloid polypeptide; PCR, polymerase chain reactions; RAGE, receptor for AGE; tPA, tissue-type plasminogen activator.

The invention provides compounds and methods for the detection and treatment of diseases associated with the excessive formation of cross-β structure.

The cross-β structure can be part of an Aβ fibril or part of another amyloid fibril. The cross-β structure can also be present in denatured proteins.

The invention provides methods to detect cross-β structure. In one embodiment, a cross-β structure-binding compound, preferably a finger domain or a molecule comprising one or more finger modules, is bound or affixed to a solid surface, preferably a microtiter plate. The solid surfaces useful in this embodiment would be known to one of skill in the art. For example, one embodiment of a solid surface is a bead, a column, a plastic dish, a plastic plate, a microscope slide, a nylon membrane, etc. After blocking, the surface is incubated with a sample. After removal of unbound sample, bound molecules comprising cross-β structure are subsequently detected using a second cross-β structure-binding compound, preferably an anti-cross-β structure antibody or a molecule containing a finger module. The second cross-β structure-binding compound is bound to a label, preferably an enzyme, such as peroxidase. The detectable label may also be a fluorescent label, a biotin, a digoxigenin, a radioactive atom, a paramagnetic ion, and a chemiluminescent label. It may also be labeled by covalent means, such as chemical, enzymatic or other appropriate means, with a moiety, such as an enzyme or radioisotope. Portions of the above-mentioned compounds of the invention may be labeled by association with a detectable marker substance (e.g., radiolabeled with ¹²⁵I or biotinylated) to provide reagents useful in detection and quantification of a compound or its receptor-bearing cells or its derivatives in solid tissue and fluid samples, such as blood, cerebral spinal fluid, urine or other. Such samples may also include serum used for tissue culture or medium used for tissue culture.

In another embodiment, the solid surface can be microspheres for, for example, agglutination tests.

In one embodiment, the compound containing a finger module is used to stain tissue samples. Preferably, the compound is fused to a protein or peptide, such as glutathion-S-transferase. Alternatively, the compound is coupled to a label. The detectable label may be a fluorescent label, a biotin, a digoxigenin, a radioactive atom, a paramagnetic ion, and a chemiluminescent label. It may also be labeled by covalent means, such as chemical, enzymatic or other appropriate means, with a moiety, such as an enzyme or radioisotope. Portions of the above-mentioned compounds of the invention may be labeled by association with a detectable marker substance (e.g., radiolabeled with ¹²⁵I, ^{99m}Tc, ¹³¹I, chelated radiolabels, or biotinylated) to provide reagents useful in detection and quantification of a compound or its receptor-bearing cells or its derivatives in solid tissue and fluid samples, such as blood, cerebral spinal fluid or urine. The compound is incubated with the sample and after washing, visualized with antibodies directed against the fused protein or polypeptide, preferably glutathion-S-transferase.

In an embodiment, the above sample is tissue from patients with or expected to suffer from a conformational disease. Alternatively, the tissue is derived from animals or from cells cultured in vitro.

The methods of the invention provide a new diagnostic tool. It was not until the present invention that a universal cross-β-structure epitope was disclosed and that a diagnostic assay could be based on the presence of the cross-β structure. Such use is particularly, useful for diagnostic identification of conformational diseases or diseases associated with amyloid formation, like AD or diabetes. It is clear that this diagnostic use is also useful for other diseases that involve cross-β structure formation, like all amyloidosis-type diseases, atherosclerosis, diabetes, thrombosis, bleeding, cancer, sepsis and other inflammatory diseases, multiple sclerosis, auto-immune diseases, disease associated with loss of memory or Parkinson and other neuronal diseases (epilepsy). For example, one can use a finger domain (of, for example, tPA) and provide it with a label (radioactive, fluorescent, etc.). This labeled finger domain can then be used either in vitro or in vivo for the detection of cross-β structure-comprising proteins, hence for determining the presence of a plaque involved in a conformational disease. One can, for example, use an ELISA to determine the amount of sepsis in a patient or one can localize a plaque involved in a conformational disease.

In another embodiment, this invention provides a method for inhibiting the formation of amyloid fibrils or any other misfolded protein assemblies or to modulate cross-β structure-induced toxicity. The compound preferably comprises a cross-β structure-binding module, preferably a finger domain, a finger domain-containing molecule, a peptidomimetic analog, and/or an anti-cross-β structure antibody, and/or a multiligand receptor or a fragment thereof.

According to the invention, the inhibition of fibril formation preferably has the consequence of decreasing the load of fibrils.

The inhibition of fibril formation or modulating cross-β structure toxicity may also have the consequence of modulating cell death. The cell can be any cell, but preferably is a neuronal cell, an endothelial cell, or a tumor cell. The cell can be a human cell or a cell from any other species.

The cell may typically be present in a subject. The subject to which the compound is administered may be a mammal, preferably a human.

The subject may be suffering from amyloidoses, from another conformational disease, from prion disease, from chronic renal failure and/or dialysis-related amyloidosis, from atheroscleroses, from cardiovascular disease, from autoimmune disease, from thrombosis and/or from bleeding, or the subject may be obese. The subject may also be suffering from inflammation, rheumatoid arthritis, diabetes, retinopathy, sepsis, disseminated intravascular coagulation (also referred to as “diffuse intravascular coagulation”), hemolytic uremic syndrome, and/or preeclampsia. The diseases that may be at least in part treated or prevented with the methods of the present invention include, but are not limited to, diabetes, AD, senility, renal failure, hyperlipidemic atherosclerosis, neuronal cytotoxicity, Down's syndrome, dementia associated with head trauma, amyotrophic lateral sclerosis, multiple sclerosis, amyloidosis, an autoimmune disease, inflammation, a tumor, cancer, male impotence, wound healing, periodontal disease, neuropathy, retinopathy, nephropathy, thrombosis, bleeding or neuronal degeneration.

The administration of compounds according to the invention may be constant or for a certain period of time. The compound may be delivered hourly, daily, weekly, monthly (e.g., in a time release form) or as a one time delivery. The delivery may also be continuous (e.g., intravenous delivery).

A carrier may be used. The carrier may be a diluent, an aerosol, an aqueous solution, a nonaqueous solution or a solid carrier. This invention also provides pharmaceutical compositions including therapeutically effective amounts of polypeptide compositions and compounds according to the invention, together with suitable diluents, preservatives, solubilizers, emulsifiers, adjuvants and/or carriers. Such compositions may be liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), antioxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts.

The administration of compounds according to the invention may comprise intralesional, intraperitoneal, intramuscular or intravenous injection; infusion; liposome-mediated delivery; topical, intrathecal, gingival pocket, per rectum, intrabronchial, nasal, oral, ocular or otic delivery. In a further embodiment, the administration includes intrabronchial administration, anal, intrathecal administration or transdermal delivery.

According to the invention, the compounds may be administered hourly, daily, weekly, monthly or annually. In another embodiment, the effective amount of the compound comprises from about 0.000001 mg/kg body weight to about 100 mg/kg body weight.

The compounds according to the invention may be delivered locally via a capsule, which allows sustained release of, the agent over a period of time. Controlled or sustained-release compositions include formulation in lipophilic depots (e.g., fatty acids, waxes, oils). Also included in the invention are particulate compositions coated with polymers (e.g., poloxamers or poloxamines) and the agent coupled to antibodies directed against tissue-specific receptors, ligands or antigens or coupled to ligands of tissue-specific receptors. Other embodiments of the compositions of the invention incorporate particulate forms with protective coatings, protease inhibitors or permeation enhancers for various routes of administration, including parenteral, pulmonary, nasal and oral.

The effective amount of the compounds according to the invention preferably comprise 1 ng/kg body weight to about 1 gr/kg body weight. The actual effective amount will be based upon the size of the compound and its properties.

The activity of tPA and/or the tPA-mediated activation of plasminogen is preferably increased by the binding to fibrin fragments, or other protein fragments that have a lysine or an arginine at the carboxy-terminal end. B-type carboxypeptidases including, but not limited to, CpB or TAFI, are enzymes that cleave off carboxy-terminal lysine and arginine residues of fibrin fragments that would otherwise bind to tPA and/or plasminogen and stimulate plasmin formation.

Because this invention has made it clear that the cross-β structures are harmful when present in certain parts of the body like, for example, the brain, and the damage is effected by the combination of cross-β structures with tPA, a method is provided to inhibit cross-β structure-mediated effects comprising providing an effective amount of a protein comprising a finger domain to block the binding sites of the cross-β structure for tPA. The cross-β structure-mediated effects may even be further diminished comprising providing an effective amount of B-type carboxypeptidase activity to inhibit the tPA activity.

The invention provides the use of a compound capable of binding to a cross-β structure for the removal of cross-β structures. The compound is a cross-β structure-binding molecule, preferably a protein and/or a functional equivalent and/or a functional fragment thereof. More preferably, the compound comprises a finger domain or a finger domain-containing molecule or a functional equivalent or a functional fragment thereof. Even more preferably, the finger domain is derived from fibronectin, FXII, HGFA or tPA. In one embodiment, the compound comprises IgIV and/or at least one chaperone like, for example, clusterin, haptoglobin, gp96, BiP, and/or an extracellular soluble fragment of a cell surface receptor like, for example, CD36, CD91, SRA, SRB-I, and RAGE. It is clear that the invention also comprises antibodies that bind cross-β structures.

In another preferred embodiment, the protein is an antibody and/or a functional equivalent, and/or a functional fragment thereof. With this use, the invention provides, for example, a therapeutic method to remove cross-β structure-comprising proteins from, for example, the circulation, preferably via extracorporeal dialysis. For example, a patient with sepsis is subjected to such use by dialysis of blood of the patient through means that is provided with, for example, preferably immobilized, finger domains. One could, for example, couple the finger domains to a solid surface or to the inside of the tubes used for dialysis. In this way, cross-β structure-comprising proteins will be removed from the bloodstream of the patient, thereby at least partly relieving patients of the negative effects caused by the cross-β structure-comprising proteins. Besides finger domain-comprising compounds, it is also possible to use other cross-β structure-binding compounds, like IgIV, chaperones, antibodies or soluble multiligand receptors (see, for instance, Tables 3-5). It is also clear that the use could be applied in hemodialysis of kidney patients.

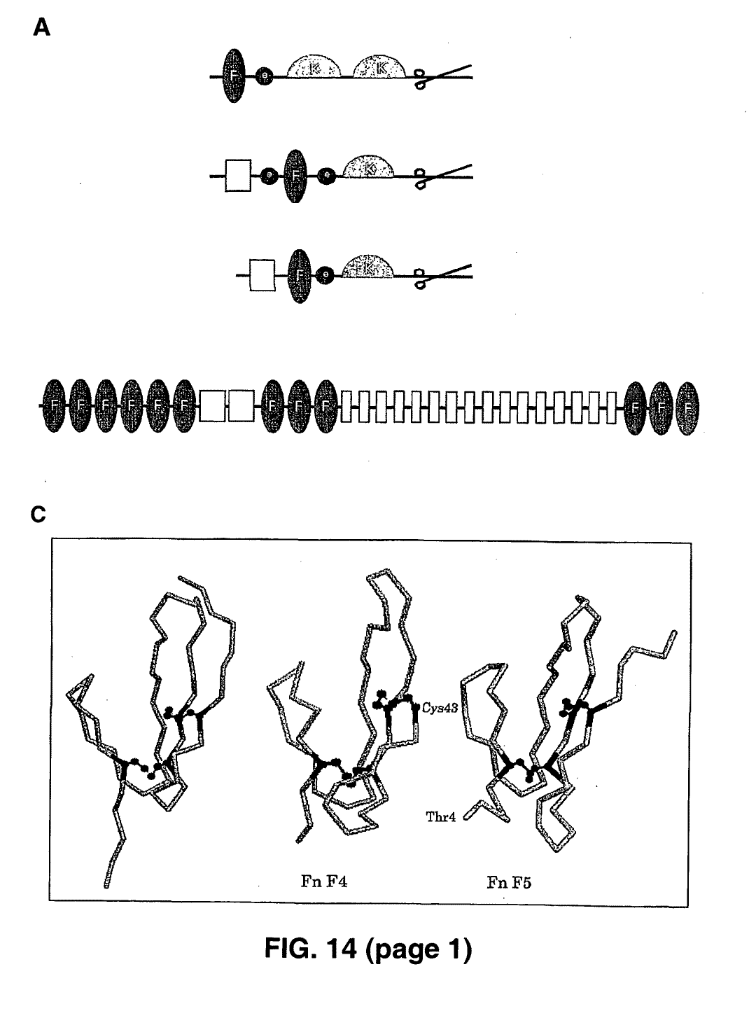

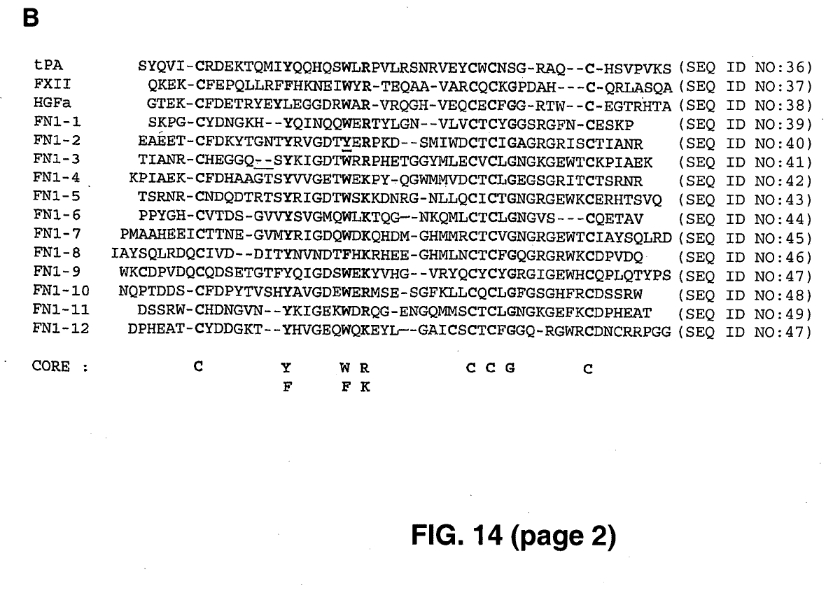

As used herein “finger” encompasses a sequence that fulfills the criteria outlined in FIG. 14. The sequence encompasses approximately 50 amino acids containing four cysteine residues at distinct spacing. Preferably, the finger domains of tPA, FXII, HGFA or fibronectin are used. Alternatively, the “finger” may be a polypeptide analog or peptidomimetic with similar function, e.g., by having three-dimensional conformation. It is feasible that such analogs have improved properties.

As mentioned before, factor XII contains a finger domain and is part of the “cross-beta structure pathway.” Factor XII is important since it activates the intrinsic coagulation pathway. Blood coagulation comprises blood clotting and/or formation of a fibrin network. Experimental evidence is provided that factor XII is activated by proteins that form crossbeta structure upon exposure to kaolin and by peptide aggregates with cross-β structure conformation. This shows that the intrinsic route of coagulation is part of the cross-β pathway and that cross-β structure compounds activate coagulation via factor XII. Moreover, experimental evidence disclosing that blood platelets become activated by cross-β structures is also provided. In this way, the cross-β structure contributes to coagulation via platelet aggregation and thrombosis. Cross-β structures also contribute to coagulation via the extrinsic pathway and thrombosis via induction of the expression of tissue factor (TF) and by stimulating its release. Cross-β structures induce TF after binding to receptors on the surface of endothelial cells, monocytes, macrophages or other cells.

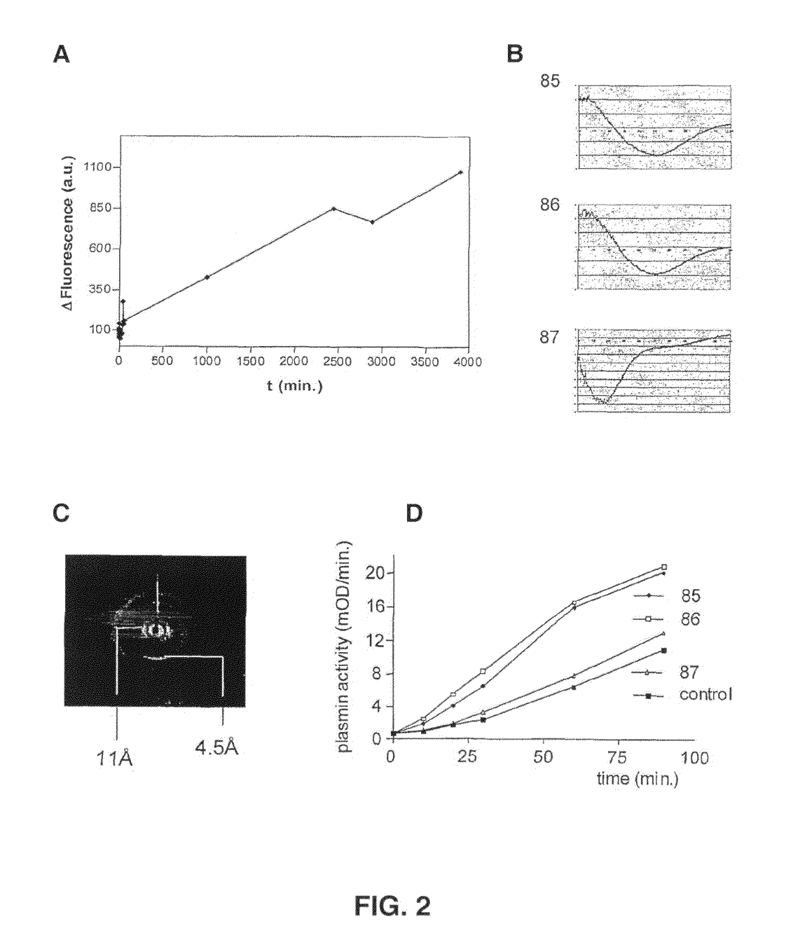

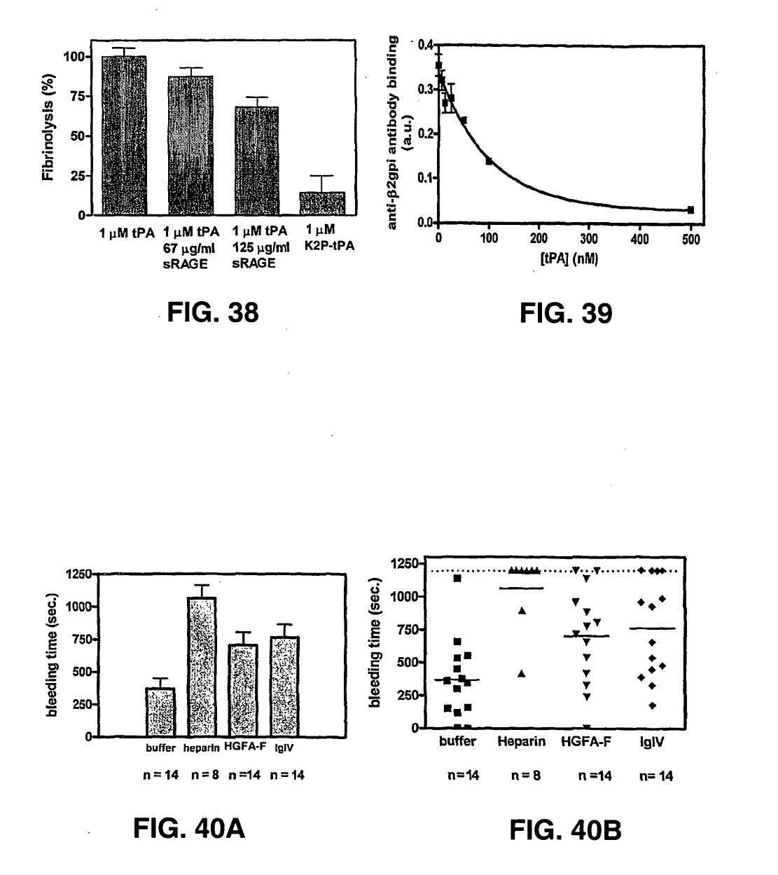

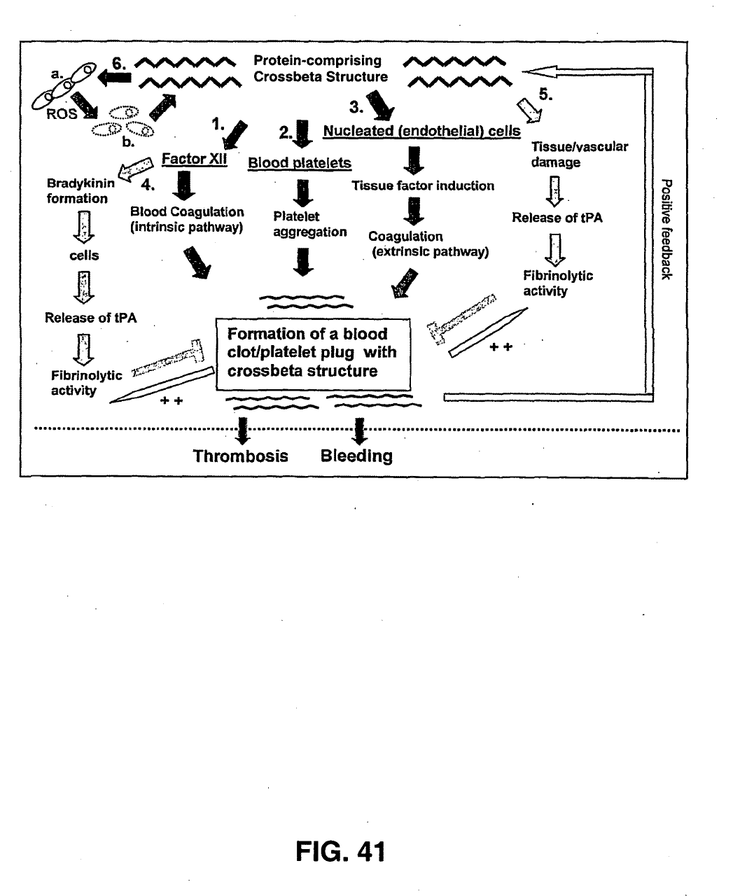

Alternatively cross-β structures induce coagulation and/or thrombosis and/or fibrinolysis and/or bleeding by the fact that fibrin, formed during blood coagulation, comprises cross-β structures, which subsequently activate blood coagulation and/or fibrinolysis. Furthermore, cross-β structures and proteinaceous molecules comprising a cross-β structure contribute to coagulation via stimulation and damage of nucleated cells and tissue. Cross-β structures and proteinaceous molecules comprising cross-β structure are furthermore capable of inducing apoptosis/necrosis of cells, which, in turn, results in formation of further cross-β structures that are capable of subsequently contributing to coagulation. FIG. 41 schematically depicts the cross-β structure-induced coagulation pathway. Taken together, it is thus concluded that cross-β structure is an important inducer of blood coagulation via, amongst other things, activation of factor XII, via activation of platelets and/or via induction of TF expression and secretion. In addition, formation of a stable fibrin blood clot is assisted by formation of fibrin polymers with crossbeta structure conformation (see, for example, FIGS. 2, 24, 37, 38 and 41). Thus, blood coagulation is part of the “cross beta pathway.” Cross-e structure binds to compounds of the blood coagulation cascade through a cross-β structure-binding domain (also referred to as “a specific binding partner” or “a specific binding part”).

Cross-beta structure in blood is provided by, for example, fibrin, glycated proteins, oxidated proteins, unfolded or misfolded proteins or pathogenic microorganisms.

Since cross-β structures are involved in the blood coagulation pathway, it is possible to interfere in blood coagulation using a binding molecule that is either capable of specifically binding a cross-β structure or a specific part of a compound, the compound being capable of specifically binding cross-β structure. One well-known blood coagulation disorder is thrombosis, involving undesired blood coagulation. Another well-known and severe coagulation syndrome is disseminated intravascular coagulation (DIC), which often occurs during, for example, sepsis. Interference with blood coagulation, for instance, enables counteracting undesired blood coagulation, such as thrombosis and/or DIC. The invention, therefore, provides a method for interfering in coagulation of blood, platelet activation, platelet aggregation and/or fibrinolysis, comprising providing to blood a binding molecule that either binds to a cross-beta structure or to a compound comprising a specific binding partner of cross-beta structure, which compound is part of a blood coagulation cascade, platelet activation cascade and/or fibrinolytic pathway. Such binding molecule is particularly suitable for the preparation of a medicament against a blood coagulation-related disease. A blood-coagulation-related disease is defined as a disease involving an undesired, harmful extent of blood coagulation. Such undesired extent of blood coagulation, for instance, causes thrombosis and/or DIC. Further provided is, therefore, a use of a binding molecule that is either capable of specifically binding to a cross-beta structure or to a compound comprising a specific binding partner of cross-beta structure, which compound is part of a blood coagulation cascade, platelet activation cascade and/or fibrinolytic pathway for the manufacture of a medicament for counteracting a blood coagulation-related disease. The disease preferably comprises thrombosis and/or bleeding and/or DIC. The term “blood” is to be understood as blood in vivo, i.e., in the circulation of a living animal (human or non-human animal) or in vitro, i.e., as blood outside of a living animal, for example, blood present in a tube. Moreover, the term blood also includes a product derived from blood but still capable of coagulating.

As depicted in FIG. 41, cross-β structure-induced blood coagulation involves, amongst other biological mechanisms, platelet activation, fibrin polymerization, expression of Tissue Factor, release of Tissue Factor, and/or activation of Tissue Factor. Blood coagulation is, therefore, influenced by influencing any one of these cascades. The invention, therefore, provides a method for interfering in coagulation of blood comprising influencing platelet activation, expression of Tissue Factor, release of Tissue Factor, and/or activation of Tissue Factor. The method is particularly suitable for influencing blood coagulation. Preferably, both platelet activation and Tissue Factor-induced blood coagulation are influenced.

One preferred embodiment further involves influencing factor XII activation. Also provided is, therefore, a method for interfering in coagulation of blood comprising influencing factor XII activation, platelet activation, expression of Tissue Factor, release of Tissue Factor, and/or activation of Tissue Factor. Preferably, factor XII activation and platelet activation are both influenced. In a further embodiment, both factor XII activation and Tissue Factor-induced blood coagulation are influenced. These embodiments are particularly suitable for influencing various routes of blood coagulation. Most preferably, factor XII activation and platelet activation and Tissue Factor-induced blood coagulation are influenced in order to, in particular, tightly regulate blood coagulation. Another preferred embodiment comprises influencing fibrin polymerization. A method according to the invention preferably furthermore comprises influencing release of intracellular tPA, and/or activation of tPA, since tPA also influences the extent of blood coagulation. In one preferred embodiment, fibrin polymerization is influenced.

In one preferred embodiment, coagulation of blood is increased. This embodiment is particularly suitable in the case of a blood coagulation deficiency, such as, for instance, in a bleeding disorder. In one particularly preferred embodiment, coagulation of blood is increased by stimulating expression and/or release of Tissue Factor by nucleated cells. According to the present invention, Tissue Factor stimulates blood coagulation. Hence, an increased amount of available Tissue Factor results in increased blood coagulation. In one embodiment, blood coagulation is increased by activating factor XII.

Blood coagulation is counteracted by tPA, since tPA converts plasminogen into plasmin; which breaks down blood clots. In one embodiment, coagulation of blood is, therefore, increased by counteracting intracellular tPA release by nucleated cells and/or tissue. Release of tPA, for instance, results from tissue damage and/or vascular damage. In order to counteract release of tPA and, hence, to increase blood coagulation, tissue damage and/or vascular damage is, therefore, preferably counteracted.

In yet another preferred embodiment, coagulation of blood is increased by activating platelets.

In yet another preferred embodiment, coagulation of blood is increased by activating thrombin and/or by supplying activated thrombin to an individual.

Hence, blood coagulation is increased by inducing and/or enhancing factor XII activation, platelet activation, fibrin polymerization, expression of Tissue Factor, release of Tissue Factor and/or activation of Tissue Factor. In one preferred embodiment, this is accomplished using a cross-β structure and/or a proteinaceous molecule comprising a cross-β structure. Further provided is, therefore, a method according to the invention, wherein factor XII activation, platelet activation, fibrin polymerization, expression of Tissue Factor, release of Tissue Factor and/or activation of Tissue Factor is induced and/or enhanced by providing a cross-beta structure and/or a proteinaceous molecule comprising a cross-beta structure. Most preferably, platelet activation and/or Tissue Factor-induced blood coagulation is induced and/or enhanced.

A use of a cross-beta structure and/or a proteinaceous molecule comprising a cross-beta structure for the manufacture of a medicament for counteracting a blood coagulation deficiency is also herewith provided.

In a further preferred embodiment, blood coagulation is decreased. This is, for instance, desired in the case of a blood coagulation-related disease, such as thrombosis and/or DIC. In one particularly preferred embodiment, coagulation of blood is decreased by counteracting expression and/or release of Tissue Factor by nucleated cells. Since Tissue Factor stimulates blood coagulation, a decreased amount of (available) Tissue Factor results in decreased blood coagulation. The invention thus provides a method according to the invention, wherein coagulation of blood is decreased by counteracting expression and/or release of Tissue Factor by nucleated cells. Preferably, intracellular tPA release by nucleated cells and/or tissue is induced and/or enhanced, since an increased amount of (available) tPA results in increased break down of blood clots and, hence, in a decreased extent of blood coagulation.

In a further embodiment, local tissue damage and/or local vascular damage is induced and/or enhanced, since tissue and/or vascular damage results in tPA release. In a particularly preferred embodiment, coagulation of blood is decreased by counteracting platelet activation.

In a further embodiment, crossbeta structure formation in fibrin is decreased by counteracting polymerization of fibrin. This is preferably performed with a crossbeta structure-binding compound such as, for example, Thioflavin, HGFA-F, sRAGE, IgIV and/or at least one chaperone.

Hence, blood coagulation is decreased by counteracting factor XII activation, platelet activation, fibrin polymerization, expression of Tissue Factor, release of Tissue Factor and/or activation of Tissue Factor. Furthermore, blood coagulation is decreased by promoting release of intracellular tPA and/or activation of tPA. In one preferred embodiment, this is accomplished using a compound that is either capable of specifically binding to a cross-β structure or to a compound comprising a specific binding partner of cross-β structure, which compound is part of a blood coagulation cascade. Further provided is, therefore, a method according to the invention, wherein factor XII activation, platelet activation, fibrin polymerization, expression of Tissue Factor, release of Tissue Factor, and/or activation of Tissue Factor is counteracted, and/or wherein release of intracellular tPA and/or activation of tPA is induced and/or enhanced by providing a compound that is either capable of specifically binding to a cross-beta structure or to a compound comprising a specific binding partner of cross-beta, which compound is part of a blood coagulation cascade. Most preferably, platelet activation and/or Tissue Factor-induced blood coagulation is counteracted.

A use of a compound that is capable of counteracting factor XII activation, platelet activation, fibrin polymerization, expression of Tissue Factor, release of Tissue Factor, and/or activation of Tissue Factor, and/or stimulating release of intracellular tPA and/or activation of tPA, for the manufacture of a medicament for counteracting a blood coagulation-related disease, is also provided. The blood coagulation-related disease preferably comprises thrombosis and/or DIC. Preferably, the compound is capable of specifically binding a platelet, fibrin, Factor XII, and/or a cell comprising Tissue Factor and/or tPA.

A binding molecule according to the invention is preferably a bi-specific molecule, i.e., a molecule with two different binding specificities. In a preferred embodiment, the bi-specific molecule is capable of binding to cross-beta, as well as to another part of the cross-beta structure and/or of a protein comprising a cross-beta structure. In another preferred embodiment, the bi-specific molecule is capable of binding to a specific binding partner, as well as to another part of the same compound, which is part of a blood-coagulating cascade. In an even more preferred embodiment of the invention, the bi-specific molecule is an antibody or a functional part and/or derivative thereof. Such a bi-specific antibody is produced as a recombinant molecule and is optionally adapted to the host in which the antibody is used.

In yet another embodiment, the binding molecule is mono-specific and binds to cross-beta structure, for example, IgIV, a chaperone like, for example, clusterin, haptoglobin, gp96, BiP, another extra-cellularly located heat-shock protein, plasminogen, and/or a cell surface receptor like, for example, CD36, CD91, SRA, SRB-I, and RAGE, Congo Red, Thioflavin T, Thioflavin S, C1q, serum amyloid P component, a finger domain-comprising protein, such as tPA, HGFA, fibronectin, Factor XII or a functional-part and/or derivative thereof, i.e., a part or derivative capable of binding to cross-beta structure.

In general, all compounds of a blood coagulation cascade that are activated through the binding of a cross-beta structure are suitable points of interfering. Preferred embodiments are a platelet or fibrin or Factor XII or a receptor present on an endothelial cell or a receptor present on a monocyte/macrophage or any other cell exposing multi-ligand receptors and comprising Tissue Factor. In one embodiment, a specific binding partner is used that is a receptor that is naturally present on a cell that is exposed to blood.

For example, blood platelets are activated after binding of a cross-beta structure. Surprisingly, activated platelets expose cross-beta structures on their surface. The cross-beta structures play a role in the coagulation cascade, for example, by enhancing activation of other platelets. The enhancement is artificially decreased by administration of a molecule binding to a cross-beta structure, such as, for instance, IgIV, a chaperone, tPA, Congo Red, RAGE, an antibody and/or Thioflavin T. This results in activated platelets that do not (or less) aggregate. Platelet activation is also accomplished by fibrin components or thrombin or small peptide compounds, for example, TRAP. These components also induce cross-beta structures on activated platelets. Preferably, activation of a platelet is accomplished by binding of a cross-beta structure to a scavenger/multi-ligand receptor like, for example, CD36, LRP, apoER2′, CD40, Toll-like receptor 4, scavenger receptor A, or scavenger receptor B-I.

Factor XII generally binds to exposed collagen at the site of vessel wall injury. Factor XII is activated by high molecular weight kininogen and kallikrein. When activated, Factor XII becomes a serine protease that activates Factor XI. Activated Factor XII also induces Bradykinin, which influences the blood flow and stimulates the release of intracellular tPA. Because cross-beta structure activates Factor XII, it initiates the blood coagulation cascade. Interfering in this pathway with, for example, the binding of a (mono- or) bi-specific molecule to a cross beta structure results in decreased blood coagulation.

Another way of interfering in a blood coagulation cascade is by interfering the binding of cross-beta structures to a receptor present on an endothelial cell or a receptor present on a monocyte/macrophage or any other cell exposing multi-ligand receptors and comprising Tissue Factor. Examples of such receptors are CD36, LRP, CD40, scavenger receptor A, scavenger receptor B-I, RAGE, FEEL-1, LOX-1, stabilin-1, and stabilin-2. Evidence is presented that at least endothelial cells and macrophages/monocytes comprise a multi-ligand receptor, as well as Tissue Factor. These examples disclose that any cell comprising the combination of a multi-ligand receptor, as well as Tissue Factor, is capable of initiating or enhancing a blood coagulation cascade.

A mono- or bi-specific molecule, as described above, is useful in the preparation of a medicament for the treatment of coagulation disorders, such as, but not limited to, thrombosis.

A pharmaceutical comprising a mono- or bi-specific molecule of the invention is very useful for treating a diverse range of blood coagulation disorders.

In another embodiment, the invention provides a method for initiating or increasing a blood-coagulating cascade by providing cross-beta structures to blood. For example, local blood clotting is induced in the case of blood loss through vascular disruption. An example of a cross-beta structure-comprising protein is fibrin.

In yet another embodiment, the invention provides a bi-specific molecule capable of binding to a specific cross-beta structure-binding part of a compound that is part of a blood-coagulating cascade, as well as to another part of the compound, or a mono- or bi-specific molecule capable of binding to cross-beta structure, as well as to another part of the same cross-beta structure. Preferably, such a mono- or bi-specific molecule is an antibody.

The invention furthermore provides a pharmaceutical comprising a mono- or bi-specific molecule as described above.

### EXPERIMENTAL PART

### Reagents

Bovine serum albumin (BSA) fraction V pH 7.0 and D-glucose-6-phosphate di-sodium (g6p), D, L-glyceraldehyde, and chicken egg-white lysozyme were from ICN (Aurora, Ohio, USA). Rabbit anti-recombinant tissue-type plasminogen activator (tPA) 385R and mouse anti-recombinant tPA 374B were purchased from American Diagnostica (Veenendaal, The Netherlands). Anti-laminin (L9393) was from Sigma. Swine anti-rabbit immunoglobulins/HRP (SWARPO) and rabbit anti-mouse immunoglobulins/HRP (RAMPO) were from DAKO Diagnostics B.V. (The Netherlands). Alteplase (recombinant tissue-type plasminogen activator, tPA) was obtained from Boehringer-Ingelheim (Germany). Reteplase (Rapilysin), a recombinant mutant tPA containing only kringle2 and the catalytic domain (K2P-tPA), was obtained from Roche, Hertfordshire, UK, and porcine pancreas carboxypeptidase B (CpB) was from Roche, Mannheim, Germany. Carboxypeptidase inhibitor (CPI) was from Calbiochem (La Jolla, Calif., USA). Tween20 was purchased from Merck-Schuchardt (Hohenbrunn, Germany). Congo red was obtained from Aldrich (Milwaukee, Wis., USA). Thioflavin T and lyophilized human hemoglobin (Rb) were from Sigma (St. Louis, Mo., USA). Lyophilized human fibrinogen was from Kordia (Leiden, The Netherlands). Chromogenic plasmin substrate S-2251 was purchased from Chromogenix (Milan, Italy). Oligonucleotides were purchased from Sigma-Genosys (U.K.). Boro glass capillaries (0.5 mm φ) were from Mueller (Berlin, Germany).

### Synthetic Peptides

Peptide Aβ (1-40), containing amino acids as present in the described human Alzheimer peptide (DAEFRHDSGYEVHHQKLVFFAEDVGSNKGAIIGLMVGGVV (SEQ ID NO:3)), fibrin peptides 85 (or FP13) (KRLEVDIDIKIRS (SEQ ID NO:4)), 86 (or FP12) (KRLEVDIDIKIR (SEQ ID NO:5)) and 87 (or FP10) (KRLEVDIDIK (SEQ ID NO:6)), derived from the sequence of human fibrin(ogen) and the islet amyloid polypeptide (IAPP) peptide or derivatives fl-hIAPP (KCNTATCATQRLANFLVHSSNNFGAILSSTNVGSNTY (SEQ ID NO:7)), ΔhIAPP(SNNFGAILSS (SEQ ID NO:8)), ΔmIAPP(SNNLGPVLPP (SEQ ID NO:9)) were obtained from Pepscan, Inc. (The Netherlands) or from the peptide synthesis facility at the Netherlands Cancer Institute (NCI, Amsterdam, The Netherlands). The peptides were dissolved in phosphate-buffered saline (PBS) to a final concentration of 1 mg ml⁻¹ and stored for three weeks at room temperature (RT) to allow formation of fibrils. During this period, the suspension was vortexed twice weekly. After three weeks, the suspension was stored at 4° C. Freeze-dried Aβ (1-40) from the NCI allowed formation of cross-β structure in the same way. Cross-β structure formation was followed in time by examination of Congo red binding and green birefringence under polarized light.

### Congo Red Binding and Thioflavin T Fluorescence of a Fibrin Clot

For Thioflavin T-fluorescence measurements, 1 mg ml⁻¹ of fibrinogen was incubated at 37° C. with 2 U ml⁻¹ of factor IIa in 150 mM NaCl, 20 mM Tris-HCl pH 7.5, 10 mM CaCl₂, and 50 μM Thioflavin T. Background fluorescence of a clot was recorded in the absence of Thioflavin T and background Thioflavin T fluorescence was measured in the absence of factor IIa. Fluorescence was measured on a Hitachi F-4500 fluorescence spectrophotometer (Ltd., Tokyo, Japan), using Sarstedt REF67.754 cuvettes. Apparatus settings: excitation at 435 nm (slit 10 nm), emission at 485 nm (slit 10 nm), PMT voltage 950 V, measuring time ten seconds, delay zero seconds. For detection of Congo red binding, a fibrin clot was formed at room temperature as described above (Thioflavin T was omitted in the buffer). The clot was incubated with Congo red solution and washed according to the manufacturer's recommendations (Sigma Diagnostics, MO, USA). The clot was analyzed under polarized light.

### Initial Preparation of Glycated Albumin, Hemoglobin (Hb) and Lysozyme

For preparation of advanced glycation end product-modified bovine serum albumin (albumin-g6p), 100 mg ml⁻¹ of albumin was incubated with PBS containing 1 M of g6p and 0.05% m/v NaN₃, at 37° C. in the dark. One albumin solution was glycated for two weeks; a different batch of albumin was glycated for four weeks. Glycation was prolonged up to 23 weeks with part of the latter batch. Human Hb at 5 mg ml⁻¹ was incubated for ten weeks at 37° C. with PBS containing 1 M of g6p and 0.05% m/v of NaN₃. In Addition, an Hb solution of 50 mg ml⁻¹ was incubated for eight weeks with the same buffer. For preparation of glyceraldehyde-modified albumin (albumin-glyceraldehyde) and chicken egg-white lysozyme (lysozyme-glyceraldehyde), filter-sterilized protein solutions of 15 mg ml⁻¹ were incubated for two weeks with PBS containing 10 mM of glyceraldehyde. In controls, g6p or glyceraldehyde was omitted in the solutions. After incubations, albumin and lysozyme solutions were extensively dialyzed against distilled water and, subsequently, stored at −20° C. Protein concentrations were determined with Advanced protein-assay reagent ADV01 (Cytoskeleton, Denver, Colo., USA). Glycation was confirmed by measuring intrinsic fluorescent signals from advanced glycation end products; excitation wavelength 380 nm, emission wavelength 435 nm.

### Further Experiment Involving Glycation

For preparation of albumin-AGE, 100 mg ml⁻¹ bovine serum albumin (fraction V, catalogue #A-7906, initial fractionation by heat shock, purity ≧98% (electrophoresis), remainder mostly globulins, Sigma-Aldrich, St. Louis, Mo., USA) was incubated at 37° C. in the dark, with phosphate-buffered saline (PBS, 140 mM sodium chloride, 2.7 mM potassium chloride, 10 mM disodium hydrogen phosphate, 1.8 mM potassium di-hydrogen phosphate, pH 7.3), 1 M D-glucose-6-phosphate disodium salt hydrate (anhydrous) (ICN, Aurora, Ohio, USA) and 0.05% (n/v) NaN₃. Bovine albumin has 83 potential glycation sites (59 lysine and 23 arginine residues, N-terminus). Albumin was glycated for two weeks (albumin-AGE:2), four weeks (albumin-AGE:4) or 23 weeks (albumin-AGE:23). In controls, g6p was omitted. After incubation, solutions were extensively dialyzed against distilled water and, subsequently, stored at 4° C. Protein concentrations were determined with advanced protein-assay reagent ADV01 (Cytoskeleton, Colo., USA). Alternatively, albumin was incubated for 86 weeks with 1 M g6p, 250 mM DL-glyceraldehyde (ICN, Aurora, Ohio, USA)/100 mM NaCNBH₃′, 1 M β-D-(−)-fructose (ICN, Aurora, Ohio, USA), 1 M D(+)-glucose (BDH, Poole, England), 500 mM glyoxylic acid monohydrate (ICN, Aurora, Ohio, USA)/1100 mM NaCNBH₃, and corresponding PBS and PBS/NaCNBH₃ buffer controls. Glycation was confirmed (i) by observation of intense brown staining, (ii) by the presence of multimers on SDS-polyacrylamide gels, (iii) by assaying binding of AGE-specific antibodies moab anti-albumin-g6p 4B5 and poab anti-fibronectin-g6p (Ph. De Groot/I. Bobbink, UMC Utrecht; unpublished data), and (iv) by measuring intrinsic fluorescent signals from AGE (excitation wavelength 380 nm, emission wavelength 445 nm). Autofluorescent signals of albumin-controls were less than 4% of the signals measured for albumin-AGE and were used for background corrections.

Isolation of Hb from Human Erythrocytes

Human Hb was isolated from erythrocytes in EDTA-anticoagulated blood of three healthy individuals and of 16 diabetic patients. 100 μl of whole blood was diluted in 5 ml of physiological salt (154 mM NaCl); cells were gently spun down, and resuspended in 5 ml of physiological salt. After a 16-hour incubation at room temperature, cells were again spun down. Pelleted cells were lysed by adding 2 ml of 0.1 M of boric acid, pH 6.5 and subsequently, cell debris was spun down. Supernatant was collected and stored at −20° C.

Determination of glycoHb Concentrations

Concentrations of glycated Hb, also named glycohemoglobin, or named HbAlc, in EDTA-blood of healthy human donors or diabetic patients, were determined using a turbidimetric inhibition immunoassay with hemolyzed whole blood, according to the manufacturer's recommendations (Roche Diagnostics, Mannheim, Germany). Standard deviations are 2.3% of the measured Hb_{Alc} concentrations.

### Binding of Congo Red to Glycated Albumin

Binding of Congo red to albumin-AGE glycated for 86 weeks with carbohydrates glucose, fructose and glucose-6-phosphate, or with carbohydrate derivatives glyceraldehyde and glyoxylic acid, was tested using air-dried samples. For this purpose, 5 μg albumin was applied to a glass cover slip and air-dried. Samples were incubated with Congo red and subsequently washed according to the manufacturer's recommendations (Sigma Diagnostics, St Louis, Mo., USA). Pictures were taken on a Leica DMIRBE fluorescence microscope (Rijswijk, The Netherlands) using 596 nm and 620 nm excitation and emission wavelengths, respectively.

### Endostatin Preparations

Endostatin was purified from Escherichia coli essentially as described.⁴⁷ In short, B121.DE3 bacteria-expressing endostatin were lysed in a buffer containing 8 M urea, 10 mM Tris (pH 8.0), 10 mM imidazole and 10 mM β-mercapto-ethanol. Following purification over Ni²⁺-aggarose, the protein sample was extensively dialyzed against H₂O. During dialysis, endostatin precipitates as a fine white solid. Aliquots of this material were either stored at −80° C. for later use, or were freeze-dried prior to storage. Soluble endostatin produced in the yeast strain Pichia pastoris was kindly provided by Dr. Kim Lee Sim (EntreMed, Inc., Rockville, Mass.). Aggregated endostatin was prepared from soluble endostatin as follows. Soluble yeast endostatin was dialyzed overnight in 8 M urea and subsequently three times against H₂O. Like bacterial endostatin, yeast endostatin precipitates as a fine white solid.

### Congo Red Staining

Freeze-dried bacterial endostatin was resuspended in either 0.1% formic acid (FA) or in dimethyl-sulfoxide and taken up in a glass capillary. The solvent was allowed to evaporate and the resulting endostatin material was stained with Congo red according to the manufacturer's protocol (Sigma Diagnostics, St. Louis, Mo., USA).

### Circular Dichroism Measurements

UV circular dichroism (CD) spectra of peptide and protein solutions (100 μg ml⁻¹ in H₂O) were measured on a JASCO J-810 CD spectropolarimeter (Tokyo, Japan). Averaged absorption spectra of five or ten single measurements from 190-240 nm or from 190-250 nm for fibrin peptides 85, 86, 87 or for albumin, glycated albumin and human Aβ(16-22), respectively, are recorded. The CD spectrum of Aβ(16-22) was measured as a positive control. Aβ(16-22) readily adopts amyloid fibril conformation with cross-β structure when incubated in H₂O. For albumin and Aβ(16-22), relative percentage of the secondary structure elements present was estimated using k2d software (Andrade, 1993).

### X-ray Fiber Diffraction

Aggregated endostatin was solubilized in 0.1% FA, lyophilized fibrin peptides were dissolved in H₂O and glycated albumin was extensively dialyzed against water. Samples were taken up in a glass capillary. The solvent was then allowed to evaporate over a period of several days. Capillaries containing the dried samples were placed on a Nonius kappaCCD diffractometer (Bruker-Nonius, Delft, The Netherlands). Scattering was measured using sealed-tube MoKα radiation with a graphite monochromator on the CCD area detector during 16 hours. Scattering from air and the glass capillary wall were subtracted using in-house software (VIEW/EVAL, Dept. of Crystal and Structural Chemistry, Utrecht University, The Netherlands).

### Transmission Electron Microscopy

Endostatin, hemoglobin and albumin samples were applied to 400 mesh specimen grids covered with carbon-coated collodion films. After five minutes, the drops were removed with filter paper and the preparations were stained with 1% methylcellulose and 1% uranyl acetate. After washing in H₂O, the samples were dehydrated in a graded series of EtOH and hexanethyldisilazane. Transmission electron microscopy (TEM) images were recorded at 60 kV on a JEM-1200EX electron microscope (JEOL, Japan).

### Enzyme-Linked Immunosorbent Assay: Binding of tPA to Glycated Albumin, Hb and Aβ(1-40)

Binding of tPA to albumin-g6p (four-weeks and 23-week incubations), albumin-glyceraldehyde, control albumin, human Hb-g6p (ten-week incubation), Hb control, or to Aβ(1-40) was tested using an enzyme-linked immunosorbent assay (ELISA) set-up. Albumin-g6p and control albumin (2.5 μg ml⁻¹ in coat buffer, 50 mM Na₂CO₃/NaHCO₃ pH 9.6, 0.02% m/v NaN3, 50 μl/well) were immobilized for one hour at room temperature in 96-well protein Immobilizer plates (Exiqon, Vedbaek, Denmark). Aβ(1-40) (10 μg ml⁻¹ in coat buffer) was immobilized for 75 minutes at room temperature in a 96-well peptide Immobilizer plate (Exiqon, Vedbaek, Denmark). Control wells were incubated with coat buffer only. After a wash step with 200 μl of PBS/0.1% v/v Tween20, plates were blocked with 300 μl of PBS/1% v/v Tween20, for two hours at room temperature, while shaking. All subsequent incubations were performed in PBS/0.1% v/v Tween20 for one hour at room temperature while shaking, with volumes of 50 μl per well. After each incubation, wells were washed five times with 200 μl of PBS/0.1% v/v Tween20. Increasing amounts of full-length tPA or K2P-tPA was added in triplicate to coated wells and to control wells. Antibody 385R and, subsequently, SWARPO, or antibody 374B and, subsequently, RAMPO, were added to the wells at a concentration of 1 μg ml⁻¹. Bound peroxidase-labeled antibody was visualized using 100 μl of a solution containing 8 mg of ortho-phenylene-diamine and 0.0175% v/v of H₂O₂ in 20 ml of 50 mM citric acid/100 mM Na₂HPO₄ pH 5.0. Staining was stopped upon adding 50 μl, of a 2-M H₂SO₄ solution. Absorbance was read at 490 nm on a Vₘₐₓ kinetic microplate reader (Molecular Devices, Sunnyvale, Calif., USA).

Competition experiments were performed with 20 or 40 nM of tPA with, respectively, albumin-g6p or Aβ(1-40) and with increasing amounts of Congo red in PBS/0.08% v/v Tween20/2% v/v EtOH.

### ELISA: Binding of tPA to Albumin-AGE

Binding of the cross-β structure-marker tPA to albumin-AGE was tested using an ELISA setup. It is shown that tPA binds to prototype amyloid peptides human Aβ(1-40) and human IAPP (this application). Therefore, tPA binding to these two peptides as positive control is used. The 86-week glycated samples and controls were coated to Greiner microlon plates (catalogue #655092, Greiner, Frickenhausen, Germany). Wells were blocked with Superblock (Pierce, Rockford, Ill., USA). All subsequent incubations were performed in PBS/0.1% (v/v) Tween20 for one hour at room temperature while shaking, with volumes of 50 μl per well. After incubation, wells were washed five times with 300 μl PBS/0.1% (v/v) Tween20. Increasing concentrations of tPA were added in triplicate to coated wells as well as to control wells. During tPA incubations of 86-week incubated samples, at least 123,000 times molar excess of ε-amino caproic acid (εACA, 10 mM) was added to the solutions. εACA is a lysine analogue and is used to avoid potential binding of tPA to albumin via its kringle2 domain. Monoclonal antibody 374b (American Diagnostica, Instrumentation laboratory, Breda, The Netherlands) and, subsequently, RAMPO (Dako Diagnostics, Glostrup, Denmark), was added to the wells at a concentration of 0.3 μg ml⁻¹. Bound peroxidase-labeled antibody was visualized using 100 μl of a solution containing 8 mg ortho-phenylene-diamine in 20 ml 50 mM citric acid/100 mM Na₂HPO₄ pH 5.0 with 0.0175% (v/v) H₂O₂. Staining was stopped upon adding 50 μl of a 2 M H₂SO₄ solution. Absorbance was read at 490 nm on a Vₘₐₓ kinetic microplate reader (Molecular Devices, CA, USA). Background signals from non-coated control wells were subtracted from corresponding coated wells.

### Initially, Thioflavin T Fluorescence of Glycated Albumin and Lysozyme, and tPA

For fluorescence measurements, 500 nM of albumin-g6p, albumin-glyceraldehyde, control albumin, lysozyme-glyceraldehyde, or control lysozyme were incubated with increasing amounts of Thioflavin T, in 50 mM of glycine-NaOH, pH 9. For blank readings, an identical Thioflavin T dilution range was prepared without protein, or Thioflavin T was omitted in the protein solutions. Samples were prepared in triplicate.

### Thioflavin T Fluorescence

In further experiments for fluorescence measurements, albumin-g6p:2, albumin-g6p:4, albumin-g6p:23 and controls in 50 mM glycine-NaOH, pH 9, were incubated with increasing amounts of ThT (Sigma-Aldrich Chemie, Steinheim, Germany), a marker for amyloid cross-β structure. Albumin-AGE:4 concentration was 175 nM; other protein concentrations were 500 nM. For fluorescence measurements with 86-week glycated samples, 140 nM of protein was incubated with a fixed concentration of 20 μM ThT. Fluorescence was measured in triplicate on a Hitachi F-4500 fluorescence spectrophotometer (Ltd., Tokyo, Japan), after one hour incubation at room temperature. Excitation and emission wavelengths were 435 nm (slit 10 nm) and 485 nm (slit 10 nm), respectively. Background signals from buffer and protein solution without ThT were subtracted from corresponding measurements with protein solution incubated with ThT.

### Fluorescence: Competitive Binding of Thioflavin T and tPA to Albumin-g6p

A solution of 430 nM albumin-g6p and 19 μM of Thioflavin T was incubated with increasing amounts of tPA for one hour at room temperature. For blank readings, albumin-g6p was omitted. Samples were prepared in four-fold in 50 mM glycine-NaOH pH 9. Emission measurements were performed as described above.

### Absorbance: Competitive Binding of Thioflavin T and tPA to Albumin-g6p

Albumin-g6p (500 nM) and Thioflavin T (10 μM) were incubated with increasing amounts of tPA, in 50 mM glycine-NaOH pH 9, for one hour at room temperature. Absorbance measurements were performed at the albumin-g6p Thioflavin T absorbance maximum at 420 nm. Samples were prepared in four-fold. For blank readings, albumin-g6p was omitted in the solutions. Absorbance was read in a quartz cuvette on a Pharmacia Biotech Ultrospec 3000 UV/visible spectrophotometer (Cambridge, England).

### Plasminogen Activation Assay

Plasminogen (200 μg ml⁻¹) was incubated with tPA (200 pM) in the presence or the absence of a cofactor (5 μM of either endostatin, Aβ(1-40) or one of the fibrin-derived peptides 85, 86 and 87). At the indicated time intervals, samples were taken and the reaction was stopped in a buffer containing 5 mM EDTA and 150 mM εACA. After collection of the samples, a chromogenic plasmin substrate S-2251 was added and plasmin activity was determined kinetically in a spectrophotometer at 37° C.

### N1E-115 Cell Culture and Differentiation

N1E-115 mouse neuroblastoma cells were routinely cultured in DMEM containing 5% FCS, supplemented with antibiotics. Cells were differentiated into post-mitotic neurons. The cells were exposed to Aβ (50 μg ml⁻¹) for 24 hours in the presence or absence of 20 μg ml⁻¹ plasminogen in the presence or absence of 50 μg ml⁻¹ CpB. Cells were photographed, counted and lysed by the addition of 4× sample buffer (250 mM Tris pH 6.8, 8% SDS, 10% glycerol, 100 mM DTT, 0.01% w/v bromophenol blue) to the medium. The lysate, containing both adherent and floating (presumably dying and/or dead) cells as well as the culture medium, were analyzed for the presence of plasminogen and plasmin, as well as for laminin, by Western blot analysis using specific antibodies against plasminogen (MoAb 3642, American Diagnostics) and laminin (PoAb L9393, Sigma).

Binding of Human Factor XII to Amyloid Peptides and Proteins that Contain the Cross-β Structure Fold

The binding of human FXII (Calbiocheem, La Jolla, Calif., USA, catalogue #233490) to amyloid (poly)peptides was tested. Prototype amyloid peptides human amyloid-β(1-40) (hAβ(1-40)) and human fibrin fragment α₁₄₇₋₁₅₉ FP13, and glucose-6-phosphate glycated bovine albumin (albumin-advanced glycation endproduct (AGE)) and glucose-6-phosphate glycated human hemoglobin (Hb-AGE), all containing cross-β structure, as well as negative controls mouse Δ islet amyloid polypeptide (ΔmIAPP), albumin-control and Hb-control, all three lacking the amyloid-specific structure, were coated to ELISA plates and overlayed with a concentration series of human factor XII. Binding of FXII was detected using a rabbit polyclonal anti-FXII antibody (Calbiochem, La Jolla, Calif., USA, catalogue #233504) and peroxidase-labeled swine anti-rabbit IgG. Wells were coated in triplicate. The FXII binding buffer consisted of 10 mM HEPES pH 7.3, 137 mM NaCl, 11 mM D-glucose, 4 mM KCl, 1 mg ml⁻¹ albumin, 50 μM ZnCl₂, 0.02% (m/v) NaN₃ and 10 mM ε-amino caproic acid (εACA). Lysine analogue εACA was added to avoid putative binding of FXII to cross-β structure via the FXII kringle domain. In addition, binding of FXII to hAβ(1-40) and the prototype amyloid human amylin fragment hΔIAPP was tested using dot blot analysis. Ten μg of the peptides that contain cross-β structure, as well as the negative control peptide MΔIAPP and phosphate-buffered saline (PBS), were spotted in duplicate onto methanol-activated nitrocellulose. Spots were subsequently incubated with 2 nM FXII in FXII buffer or with FXII buffer alone, anti-FXII antibody, and SWARPO. Binding of FXII was visualized by chemiluminescence upon incubation with enhanced luminol reagent (PerkinElmer Life Sciences, Boston, Mass., USA). To test whether FXII and tPA, which are known for their capacity to bind to polypeptides that contain the cross-β structure fold, bind to overlapping binding sites on amyloid (poly)peptides, competitive ELISAs were performed. Coated hAβ(1-40) or amyloid albumin-AGE were incubated with 2.5 nM or 15 nM FXII in binding buffer, in the presence of a concentration series of human recombinant tissue-type plasminogen activator (Actilyse®, full-length tPA), or Reteplase® (K2P-tPA). Reteplase is a truncated form of tPA that consists of the second kringle domain and the protease domain. The full-length tPA and K2P-tPA concentration was, at maximum, 135 times the k_{D} for tPA binding to hβ(1-40) (50 nM) or 150 times the k_{D} for tPA binding to albumin-AGE (1 nM).

### Cloning Procedure

Cloning of the amino-terminal finger domain (F) of human tPA, residues Ser1-Ser50, preceded by the pro-peptide (residues Met-35-Arg-1) and a BglII restriction site, was performed by using PCR and standard recombinant DNA techniques. In brief, the propeptide-finger region was amplified by PCR using 1 ng of plasmin Zp17, containing the cDNA encoding tPA as a template (Johannessen, 1990). Oligonucleotides used were 5′AAAAGTCGACAGCCGCCACCATGGATGCAATGAAGAGA (1) (SEQ ID NO:10) and 3′AAAAGCGGCCGCCCACTTTTGACAGGCACTGAG (2) (SEQ ID NO:11) comprising a SalI- or a NotI restriction-site, respectively (underlined). The PCR product was cloned in a SalI/NotI-digested expression vector, pMT2-GST (Gebbink, 1995). As a result, a construct is generated that contains a SalI restriction site, the coding sequence for the finger domain of tPA, a NotI and a KpnI restriction site, a thrombin cleavage-site (TCS), a glutathion-S-transferase (GST) tag and an EcoRI restriction site. The appropriate sequence of the construct was confirmed by sequence analysis. In a similar way, a construct consisting of the tPA-F-EGF domains was prepared. Next, the constructs were ligated SalI-EcoRI in pGEM3Zf(−) (Promega, Madison, Wis., USA). The HindIII-SalI-tPA propeptide-BglII-F-NotI-KpnI-TCS-GST-EcoRI construct was used as a cloning cassette for preparation of constructs containing tPA K1, F-EGF-K1, EGF, as well as human hepatocyte growth factor activator F and F-EGF, human factor XII F and F-EGF, and human fibronectin F4, F5, F4-5 and F10-12. Subsequently, constructs were ligated HindIII-EcoRI in the pcDNA3 expression vector (Invitrogen, Breda, The Netherlands). In addition, the GST tag alone was cloned into pcDNA3, preceded by the tPA propeptide. Primers used for constructs were:

tPA-F-EGF

(SEQ ID NO: 12)

3′AAAAGCGGCCGCGTGGCCCTGGTATCTATTTC (3) and (1)
tPA EGF

(SEQ ID NO: 13)

5′AAAAGAGATCTGTGCCTGTCAAAAGTTGC (4) and (2)
tPA K1

(SEQ ID NO: 14)

5′AAAAGAGATCTGATACCAGGGCCACGTGCTAC (5)

(SEQ ID NO: 15)

3′AAAAGCGGCCGCCCGTCACTGTTTCCCTCAGAGCA (6)
tPA-F-EGF-K1
(1) and (6)
GST tag

(SEQ ID NO: 16)

(1) and AAAAGCGGCCGCCTGGCTCCTCTTCTGAATC (7)
Fibronectin F4

(SEQ ID NO: 17)

5′TGCAAGATCTATAGCTGAGAAGTGTTTTGAT (8)

(SEQ ID NO: 18)

3′GATGCGGCCGCCCTGTATTCCTAGAAGTGCAAGTG (9)
Fibronectin F5

(SEQ ID NO: 19)

5′TGCAAGATCTACTTCTAGAAATAGATGCAAC (10)

(SEQ ID NO: 20)

3′TGATGCGGCCGCCCCACAGAGGTGTGCCTCTC (11)
Fibronectin F4-5
(8) and (11)
Fibronectin F10-12

(SEQ ID NO: 21)

5′AAAAAAGATCTAACCAACCTACGGATGACTC (12)

(SEQ ID NO: 22)

3′AAAAAAGGTACCGACTGGGTTCACCCCCAGGT (13)
factor XII F

(SEQ ID NO: 23)

5′GAAACAAGATCTCAGAAAGAGAAGTGCTTTGA (14)

(SEQ ID NO: 24)

3′ACGGGCGGCCGCCCGGCCTGGCTGGCCAGCCGCT (15)
factor XII F-EGF

(SEQ ID NO: 25)

5′AAAAAAGATCTCAGAAAGAGAAGTGCTTTGA (16)

(SEQ ID NO: 26)

3′AAAAAGGTACCGGCTTGCCTTGGTGTCCACG (17)
HGFa-F

(SEQ ID NO: 27)

5′GCAAGAAGATCTGGCACAGAGAAATGCTTTGA (18)

(SEQ ID NO: 28)

3′AAGGGCGGCCGCCCAGCTGTATGTCGGGTGCCTT (19)
HGFA-F-EGF

(SEQ ID NO: 29)

5′AAAAAAGATCTGGCACAGAGAATGCTTTGA (20)

(SEQ ID NO: 30)

3′AAAAAGGTACCGCTCATCAGGCTCGATGTTG (21)

Transient Expression of tPA-F-GST in 293T Cells

Initially, 293T cells were grown in RPMI1640 medium (Invitrogen, Scotland, U.K.) supplemented with 5% v/v fetal calf-serum, penicillin, streptomycin and guanidine, to 15% confluency. Cells were transiently transfected using Fugene-6, according to the manufacturer's recommendations (Roche, Ind., USA). pMT2-tPA-F-GST containing the tPA fragment, or a control plasmid, pMT2-RPTPμ-GST, containing the extracellular domain of receptor-like protein tyrosine phosphatase μ (RPTPμ), were transfected, and medium was harvested after 48 hours transfection. Expression of tPA-F-GST and RPTPμ-GST in 293T medium was verified by immunoblotting. Collected samples were run out on SDS-PAA gels after the addition of 2× sample buffer. Gels were blotted on nitrocellulose membranes. Membranes were blocked in 1% milk (Nutricia) and incubated with primary monoclonal anti-GST antibody 2F3 and secondary HRP-conjugated rabbit anti-mouse IgG (RAMPO). The blots were developed using Western Lightning Chemiluminescence Reagent Plus (PerkinElmer Life Sciences, MA, USA).

### Stable Expression of Finger Constructs in BHK Cells

Baby hamster kidney cells were seeded in DMEM/NUT mix F-12(HAM) medium (Invitrogen, U.K.) supplemented with 5% v/v fetal calf-serum (FCS), penicillin, streptomycin and guanidine, to 1% confluency. Cells were stably transfected by using the Ca₃(PO₄)₂-DNA precipitation method. After 24 hours, medium was supplemented with 1 mg ml⁻¹ geneticin G-418 sulphate (Gibco, U.K.). Medium with G-418 was refreshed several times over ten days to remove dead cells. After this period of time, stable single colonies were transferred to new culture flasks and cells were grown to confluency. Expression of constructs was then verified by immunoblotting. Collected samples were run out on SDS-PAA gels after the addition of 2× sample buffer. Gels were blotted on nitrocellulose membranes. Membranes were blocked in 5% milk (Nutricia) with 1.5% m/v BSA and incubated with primary monoclonal anti-GST antibody (Santa Cruz Biotechnology, Santa Cruz, Calif., USA, catalogue #Z-5), and secondary HRP-conjugated rabbit anti-mouse IgG (RAMPO). The blots were developed using Western Lightning. Chemiluminescence Reagent Plus (PerkinElmer Life Sciences, MA, USA). Stable clones were from now on grown in the presence of 250 μg ml⁻¹ G-418. For pull-down experiments, conditioned medium with 5% FCS of stable clones that produce constructs of interest was used. For purification purposes, cells of a stable clone of tPA-F-EGF-GST were transferred to triple-layered culture flasks and grown in medium with 0.5% v/v Ultroser G (ITK Diagnostics, Uithoorn, The Netherlands). Medium was refreshed every three to four days. tPA-F-EGF-GST was isolated from the medium on a Glutathione Sepharose 4B (Amersham Biosciences, Uppsala, Sweden) column and eluted with 100 mM reduced glutathione (Roche Diagnostics, Mannheim, Germany). Purity of the construct was checked with SDS-PAGE followed by Coomassie staining or Western blotting. From these analyses, it, is clear that some QST is present in the preparation. Purified tPA-F-EGF-GST was dialyzed against PBS and stored at −20° C.

Purification of GST-Tagged tPA-F-GST and RPTPμ-GST

Medium was concentrated twenty-fold using Nanosep 10K Ω centrifugal devices (Pall Gelman Laboratory, MI, USA) and incubated with glutathione coupled to Sepharose 4B, according to the manufacturer's recommendations (Pharmacia Biotech, Uppsala, Sweden). Bound constructs were washed with PBS and eluted with 10 mM of glutathione in 50 mM Tris-HCl pH 8.0. Constructs were stored at −20° C., before use.

### Amyloid Pull-Down

Conditioned medium of BHK cells expressing GST-tagged tPA-F, F-EGF, EGF, K1, F-EGF-K1, FXII F, HGFa-F, Fn F4, Fn F5, Fn F4-5 and GST was used for amyloid-binding assays. At first, constructs were adjusted to approximately equal concentration using Western blots. Qualitative binding of the recombinant fragments are evaluated using a “pull-down” assay. To this end, the recombinantly made fragments are incubated with either Aβ or IAPP fibrils. Since these peptides form insoluble fibers, unbound proteins can be easily removed from the fibers following centrifugation. The pellets containing the bound fragments are subsequently washed several times. Bound fragments are solubilized in SDS-sample buffer and analyzed by PAGE, as well as unbound proteins in the supernatant fraction and starting material. The gels are analyzed using immunoblotting analysis with the anti-GST antibody Z-5.

Amyloid ELISA with tPA-F-EGF-GST

In order to define the affinity of the purified tPA-F-EGF-GST recombinant protein, ELISAs with immobilized amyloid (poly)peptides and non-amyloid control ΔmIAPP were performed. Twenty-five μg ml⁻¹ of Aβ, FP13, IAPP or ΔmIAPP was immobilized on Exiqon peptide immobilizer plates. A concentration series of tPA-F-EGF-GST in the presence of excess εACA, was added to the wells and binding was assayed using anti-GST antibody Z-5. As a control, GST (Sigma-Aldrich, St. Louis, Mo., USA, catalog #G-5663) was used at the same concentrations.

Immunohistochemistry: Binding of tPA-F-EGF to Human AD Brain

Paraffin brain sections of a human afflicted with AD was a kind gift of Prof. Slootweg (Dept. of Pathology, UMC Utrecht). Sections were deparaffinized in a series of xylene-ethanol. Endogenous peroxidases were blocked with methanol/1.5% H₂O₂ for 15 minutes. After rinsing in H₂O, sections were incubated with undiluted formic acid for ten minutes, followed by incubation in PBS for five minutes. Sections were blocked in 10% HPS in PBS for 15 minutes. Sections were exposed for two hours with 7 nM of tPA-F-EGF-GST or GST in PBS/0.3% BSA. After three wash steps with PBS, sections were overlayed with 200 ng ml⁻¹ anti-GST antibody Z-5 for one hour. After washing, ready-to-use goat anti-rabbit Powervision (Immunologic, Duiven, The Netherlands, catalogue #DPVR-5SAP) was applied and incubated for one hour. After washing, sections were stained for ten minutes with 3,3′-diamino benzidine (Sigma-Aldrich, St Louis, Mo., USA, catalogue #D-5905), followed by hematoxylin staining for ten seconds. After washing with H₂O, sections were incubated with Congo red according to the manufacturer's recommendations (Sigma Diagnostics, St. Louis, Mo., USA). Sections were cleared in xylene and mounted with D.P.X. Mounting Medium (Nustain, Nottingham, U.K.). Analysis of sections was performed on a Leica DMIRBE fluorescence microscope (Rijswijk, The Netherlands). Fluorescence of Congo red was analyzed using an excitation wavelength of 596 nm and an emission wavelength of 620 nm.

ELISA: Binding of tPA-F-GST and RPTPμ-GST to Human Aβ (1-40) and Glycated Albumin

Binding of tPA-F-GST and RPTPμ-GST to fibrous amyloids human Aβ (1-40) and albumin-g6p was assayed with an ELISA. In brief, human Aβ (1-40), albumin-g6p, or buffer only, were coated on a peptide I Immobilizer, or a protein I Immobilizer, respectively. Wells were incubated with the purified GST-tagged constructs or control medium, and binding was detected using primary anti-GST monoclonal antibody 2F3 and RAMPO. The wells were also incubated with 500 nM of tPA in the presence of 10 mM of εACA. Binding of tPA is then independent of the lysyl-binding site located at the kringle2 domain. Binding of tPA was measured using primary antibody 374B and RAMPO. Experiments were performed in triplicate and blank readings of non-coated wells were subtracted.

### Anti-AGE Antibodies

Antibodies against glucose-6-phosphate glycated bovine serum albumin were elicited in rabbits using standard immunization schemes. Anti-AGE1 was obtained after immunization with two-week glycated albumin-AGE (Prof. Dr. Ph.G. de Groot/Dr. I. Bobbink; unpublished data). The antibody was purified from serum using a Protein G column. Anti-AGE2 was developed by Davids Biotechnologie (Regensburg, Germany). After immunization with albumin-AGE:23, antibodies were affinity purified on human Aβ(1-40) conjugated to EMD-Epoxy activated beads (Merck, Darmstadt, Germany). Polyclonal mouse anti-AGE antibody was obtained after immunization with albumin-AGE:23 and human Aβ(1-40), in a molar ratio of 9:1. Polyclonal serum was obtained using standard immunization procedures, which were performed by the Academic Biomedical Cluster Hybridoma Facility (Utrecht University, The Netherlands). Subsequently, monoclonal antibodies were generated using standard procedures.

### ELISA: Binding of Antibodies Against Amyloid Peptides or Glycated Protein to Protein-AGE and Amyloid Fibrils

For ELISAs, amyloid compounds were immobilized on Exiqon peptide or protein Immobilizers (Vedbaek, Denmark), as described before. Anti-AGE antibodies and commercially available anti-Aβ(1-42) H-43 (Santa Cruz Biotechnology, Santa Cruz, Calif., USA) were diluted in PBS with 0.1% v/v Tween20. Rabbit anti-human vitronectin K9234 was a kind gift of Dr. H. de Boer (UMC Utrecht), and was used as a negative control. For ELISAs with mouse polyclonal anti-albumin-AGE/Aβ, control serum with antibody elicited against an unrelated protein was used. Binding of mouse polyclonal anti-albumin-AGE/Aβ was performed using a dilution series of serum in PBS/0.1% Tween20. For competitive binding assays with IAPP, anti-AGE1 was pre-incubated with varying IAPP concentrations. The IAPP fibrils were spun down and the supernatant was applied in triplicate to wells of an ELISA plate coated with Aβ. Competitive binding assays with multiligand cross-β structure-binding serine protease tPA were performed in a slightly different way. Coated Aβ and IAPP are overlayed with an anti-AGE1 or anti-Aβ(1-42) H-43 concentration related to the k_{D}, together with a concentration series of tPA. A 10⁷-10⁴ times molar excess of lysine analogue εACA (10 mM) was present in the binding buffer in order to avoid binding of tPA to lysine residues of Aβ and IAPP, which would be independent of the presence of amyloid structures.

Pull-Down Assay with Amyloid Peptides and Rabbit Anti-AGE1 Antibody

Anti-AGE1 was incubated with amyloid aggregates of Aβ(16-22), Aβ(1-40) and IAPP. After centrifugation, pellets were washed three times with PBS/0.1% Tween20, dissolved in non-reducing sample buffer (1.5% (m/v) sodium dodecyl sulphate, 5% (v/v) glycerol, 0.01% (m/v) bromophenol blue, 30 mM Tris-HCl pH 6.8). Supernatant after pelleting of the amyloid fibrils was diluted 1:1 with 2× sample buffer. Samples were applied to a polyacrylamide gel and after Western blotting, anti-AGE1 was detected with SWARPO.

Immunohistochemical Analysis of the Binding of Anti-AGE2 to an Amyloid Plaque in a Section of a Human Brain Afflicted with AD

Rabbit anti-AGE2, affinity purified on an Aβ column, was used for assaying binding properties towards amyloid plaques in brain sections of a human with Aβ. The procedure was performed essentially as described above. To avoid eventual binding of 11 μg ml⁻¹ anti-AGE2 to protein-AGE adducts or to human albumin in the brain section, 300 nM of g6p-glycated dipeptide Gly-Lys was added to the binding buffer, together with 0.3% m/v BSA. After the immunohistochemical stain, the section was stained with Congo red.

### Sandwich ELISA for Detection of Amyloid Albumin-AGE in Solution

For detection of amyloid cross-β structure in solutions, the sandwich ELISA approach was used. Actilyse tPA was immobilized at a concentration of 10 μg ml⁻¹ to wells of a 96-well protein Immobilizer plate (Exiqon, Vedbaek, Denmark). Concentration series of albumin-AGE:23 and albumin-control:23 were added to the tPA-coated wells, as well as to non-coated control wells. Binding of amyloid structures was detected upon incubation with 1 μg ml⁻¹ anti-Aβ(1-42) H-43 (Santa Cruz Biotechnology, Santa Cruz, Calif., USA) and subsequently 0.3 μg ml⁻¹ SWARPO, followed by ortho-phenylene-diamine/H₂O₂/H₂SO₄ stain.

### Preparation of Cross-β Structure-Rich Compounds

Soluble endostatin produced in the yeast strain Pichia pastoris was kindly provided by Dr. Kim Lee Sim (EntreMed, Inc., Rockville, Mass., USA). Cross-β structure-rich endostatin was prepared from soluble endostatin as follows. Soluble yeast endostatin was dialyzed overnight in 8 M urea and subsequently three times against H₂O. Endostatin precipitates as a fine white solid. The presence of cross-β structure was established by Congo red binding and X-ray fiber diffraction (Kranenburg, 2002; Kranenburg, 2003). Aggregated endostatin was solubilized in 0.1% formic acid, lyophilized fibrin peptides and Aβ were dissolved in H₂O and glycated albumin was extensively dialyzed against water. Samples were taken up in a glass capillary. The solvent was then allowed to evaporate over a period of days. Capillaries containing the dried samples were placed on a Nonius kappaCCD diffractometer (Bruker-Nonius, Delft, The Netherlands). Scattering was measured using sealed-tube MoKα radiation with a graphite monochromator on the CCD area detector during 16 hours. Scattering from air and the glass capillary wall were subtracted using in-house software (VIEW/EVAL, Dept. of Crystal- and Structural Chemistry, Utrecht University, The Netherlands).

For preparation of advanced glycation end product-modified bovine serum albumin (albumin-AGE), 100 mg ml⁻¹ of albumin was incubated with PBS containing 1 M of glucose-6-phosphate (g6p) and 0.05% m/v NaN₃, at 37° C. in the dark. Glycation was prolonged up to 23 weeks (Bouma, 2003). Human hemoglobin (Hb) at 5 mg ml⁻¹ was incubated for 32 weeks at 37° C. with PBS containing 1 M of g6p and 0.05% m/v of NaN₃. In control solutions, g6p was omitted. After incubations, solutions were extensively dialyzed against distilled water and, subsequently, stored at 4° C.

Protein concentrations were determined with Advanced protein-assay reagent ADV01 (Cytoskeleton, Denver, Colo., USA). Glycation and formation of advanced glycation end products (AGE) was confirmed by measuring intrinsic fluorescent signals from advanced glycation end products; excitation wavelength 380 nm, emission wavelength 435 nm. In addition, binding of AGE-specific antibodies was determined. Presence of cross-β structure in albumin-AGE was confirmed by Congo red binding, Thioflavin T binding, the presence of β-sheet secondary structure, as observed with circular dichroism spectropolarimetry (CD) analyses, and by X-ray fiber diffraction experiments (Bouma, 2003). Presence of cross-β structure in Hb-AGE was conformed by tPA binding, CD analyses, transmission electron microscopy imaging of fibrillar structures and by Congo red fluorescence measurements. Amyloid preparations of, human γ-globulins were made as follows. Lyophilized γ-globulins (Sigma-Aldrich) was dissolved in a 1(:)1 volume ratio of 1,1,1,3,3,3-hexafluoro-2-propanol and trifluoro-acetic acid and subsequently dried under an air stream. Dried γ-globulins were dissolved in H₂O to an end concentration of 1 mg/ml and kept at room temperature for at least three days. Aliquots were stored at −20° C. and analyzed for the presence of cross-β structure. Fluorescence of Congo red and Thioflavin T was assessed, as well as tPA binding, in an ELISA and tPA activating properties in the chromogenic plasmin assay.

Other peptide batches with amyloid-like properties were prepared as follows: Human Aβ (1-40) Dutch type (DAEFRHDSGYEVHHQKLVFFAEDVGSNKGAIIGLMVGGVV (SEQ ID NO:3)), islet amyloid polypeptide (IAPP, KCNTATCATQRLANFLVHSSNNFGAILSSTNVGSNTY (SEQ ID NO:7)), amyloid: fragment of transthyretin (TTR11, YTIAALLSPYS (SEQ ID NO:31)), laminin α1-chain(2097-2108) amyloid core peptide (LAM12, AASIKVAVSADR (SEQ ID NO:32)), mouse non-amyloidogenic IAPP(20-29) core (mIAPP, SNNLGPVLPP (SEQ ID NO:9)), non-amyloid fragment FP10 of human fibrin α-chain(148-157) (KRLEVDIDIK (SEQ ID NO:6)) (Kranenburg, 2002) and human fibrin α-chain(148-160) amyloid fragment with Lys157Ala mutation (FP13, KRLEVDIDIAIRS (SEQ ID NO:33)) (Kranenburg, 2002). For pull-down experiments, Aβ and IAPP were dissolved in PBS at 1 mg ml⁻¹ and kept at room temperature for at least three weeks. Alternatively, amyloid Aβ, IAPP, FP13 and LAM12 were disaggregated in a 1:1 (v/v) mixture of 1,1,1,3,3,3-hexafluoro-2-isopropyl alcohol and trifluoroacetic acid, air-dried and dissolved in H₂O (Aβ, IAPP, LAM12: 10 mg ml⁻¹, FP13: 1 mg ml⁻¹). After three days at 37° C., peptides were kept at room temperature for two weeks, before storage at 4° C. Freshly dissolved Aβ (10 mg ml⁻¹) in 1,1,1,3,3,3-hexafluoro-2-isopropyl alcohol and trifluoroacetic acid was diluted in H₂O prior to immobilization on ELISA plates. TTR11 (15 mg ml⁻¹) was dissolved in 10% (v/v) acetonitrile in water, at pH 2 (HCl), and kept at 37° C. for three days and subsequently at room temperature for two weeks. mIAPP and FP10 were dissolved at a concentration of 1 mg ml⁻¹ in H₂O and stored at 4° C. Peptide solutions were tested for the presence of amyloid conformation by Thioflavin T (ThT) or Congo red fluorescence as described (Bouma, 2003). ThT and Congo red fluorescence was enhanced for amyloid peptides, and not for non-amyloid mIAPP, FP10 or freshly dissolved Aβ.

### Plasmin-α2-Anti-Plasmin and Factor XIIa Measurements

Factor XIIa and PAP levels were measured in citrated plasma of 40 apparently healthy controls and of 40 patients with systemic amyloidosis. Factor XIIa was measured with an ELISA (Axis-Shield Diagnostics, Dundee, UK). PAP complexes were measured with the ELISA of Technoclone (Vienna, Austria). The control group consisted of 19 male and 21 female subjects with an average age of 49.4 years (standard deviation 6.8 years). The patient population consisted of 17 male and 23 female subjects with an average age of 51.8 years (standard deviation 9.9 years). Patients' diagnosis was biopsy proven. All patients have provided informed consent prior to inclusion in this study and the study was approved by the local ethical committee.

### Cloning and Expression of Recombinant Fibronectin Type I Domains

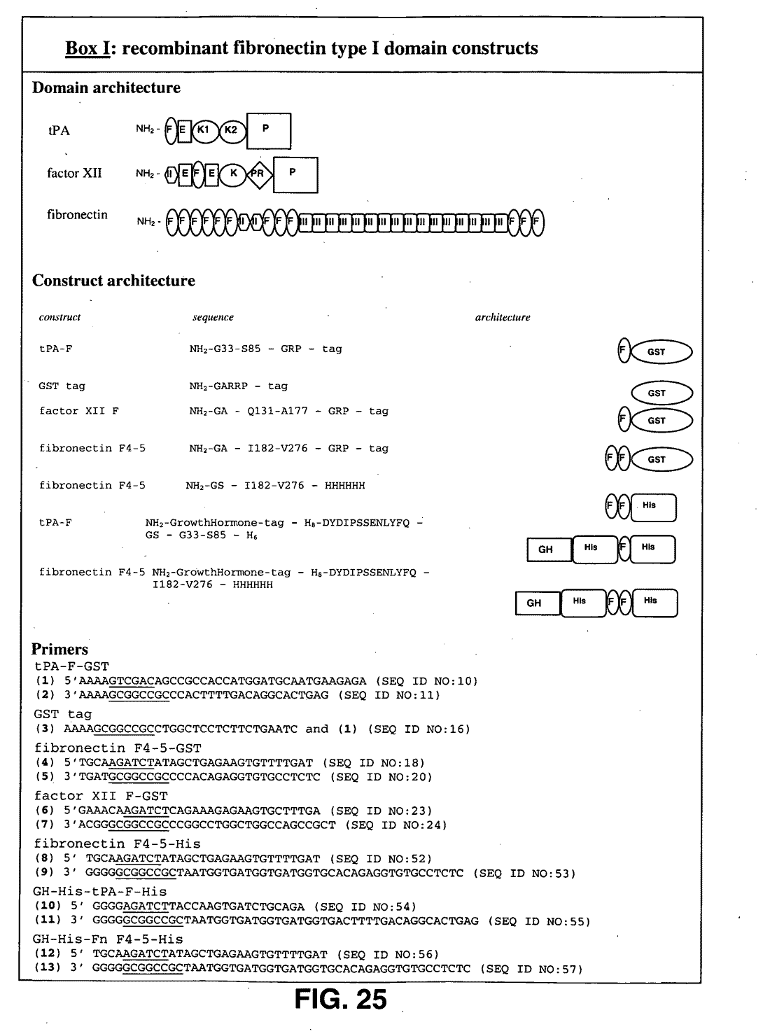

Amino acid sequences of recombinantly produced domains of tPA, fibronectin and factor XII, and the domain architecture of the recombinant constructs are depicted in FIG. 25. Amino acid residue numbering is according to SwissProt entries. Each construct has a carboxy terminal GST-tag (GST). Factor XII fibronectin type I domain (F) and fibronectin F4-5 are preceded by two amino acids (GA), following the C-terminus of the tPA propeptide. All F constructs are followed by the (G)RP sequence derived from the original pMT2-GST vector. For each recombinant construct, the oligonucleotides that were used for PCR are listed in FIG. 25. The relevant restriction sites are underlined. The tPA fibronectin type I domain (F, finger domain), together with the tPA propeptide, was amplified using 1 ng vector Zp17 containing tPA and oligonucleotides 1 and 2, digested with SalI and NotI and cloned into pMT2SM-GST. As a result, Schistosoma japonicum glutathion-S-transferase (GST) is fused to the C-terminus of the expressed constructs. The constructs were subsequently ligated with SalI and EcoRI in pGEM3Zf(−) (Promega, Madison, Wis., USA). The resulting plasmid was used as a cloning cassette for preparation of factor XII F and fibronectin F4-5 constructs.

The selection of fibronectin type I domains of fibronectin was based on the following reasoning. tPA binds to fibrin with its fibronectin type I domain and competes with fibronectin for fibrin binding. A fibrin binding site of fibronectin is enclosed in its fibronectin type I 4-5. It is shown here that the fibronectin type I domain of tPA mediates binding to amyloid. This also suggests that the fibrin-binding fibronectin type I domains of fibronectin can bind to amyloid. All domains were cloned after the tPA propeptide using a BglII restriction site that is present between the tPA propeptide region and the F domain (Johannessen, 1990), and the NotI or KpnI site that is present in front of the thrombin cleavage site (Gebbink, 1995). Subsequently, constructs were ligated HindIII and EcoRI in the pcDNA3.1 expression vector (Invitrogen, The Netherlands). This results in, e.g., pcDNA3.1-factor XII F-GST and pcDNA3.1-Fn F4-S-GST. In addition, the GST tag alone, preceded by the tPA propeptide, was cloned into pcDNA3.1. The separate GST-tag has five additional residues at the N-terminus (GARRP). tPA cDNA was a kind gift of M. Johannessen (NOVO Research Institute, Bagsvaerd, Denmark). The cDNA encoding for factor XII was a kind gift of F. Citarella (University of Rome, La Sapienza, Italy). S. A. Newman (New York Medical College, Valhalla, USA) kindly provided the cDNA encoding for an N-terminal fragment of human fibronectin, comprising fibronectin type I domains 4-5.

Alternatively, recombinant finger domains of fibronectin (F4-5) and tPA were expressed with a His-tag. Two fibronectin F4-5 constructs were cloned; one construct comprising the Ig_{κ} signal sequence (vector 71, ABC-expression facility, Utrecht University/UMC Utrecht). With two designed primers (8, 9, see FIG. 25), the fibronectin fragment was obtained from the construct pcDNA3.1-Fn F4-S-GST and BamHI and NotI restriction sites were introduced at the termini. In addition, cDNA encoding for a C-terminal His-tag was included in the designed primer. The cDNA fragment was cloned BglII-NotI in vector 71 that was digested with BamHI-NotI. Vector 71 has a BamHI site next to the Ig_{κ} signal sequence. See FIG. 25 for the construct details. A construct comprising the signal sequence of human growth hormone, the cDNA encoding for growth hormone (GH), an octa-His tag, a TEV cleavage site, the tPA-F insert and a C-terminal hexa-His tag was made using vector 122b (ABC-expression facility). The tPA-F-His cDNA was obtained using pcDNA3.1-tPA-F-GST as a template for a PCR with primers 10 and 11 (FIG. 25). The PCR insert was digested BglII-NotI; the vector was digested BamHI-NotI. The BamHI site is located next to the GH-His-TEV sequence. A second Fn F4-5 construct was made similarly to the GH-His-tPA-F-His construct (see FIG. 25).

tPA/Plasminogen Activation Assay And Factor XII Activation Assay

Plasmin activity was assayed as described (Kranenburg, 2002). Peptides and proteins that were tested for their stimulatory ability were regularly used at 100 μg ml⁻¹. The tPA and plasminogen concentrations were 200 pM and 1.1 μM, respectively. Chromogenic substrate S-2251 (Chromogenix) was used to measure plasmin activity. Conversion of inactive zymogen factor. XII to proteolytically active factor XII (factor XIIa) was assayed by measurement of the conversion of chromogenic substrate Chromozym-PK (Roche Diagnostics, Almere, The Netherlands) by kallikrein. Chromozym-PK was used at a concentration of 0.3 mM. Factor XII, human plasma prekallikrein (Calbiochem) and human plasma cofactor high-molecular weight kininogen (Calbiochem) were used at concentrations of 1 μg ml⁻¹. The assay buffer contained HBS (10 mM HEPES, 4 mM KCl, 137 mM NaCl, 5 μM ZnCl₂, 0.1% m/v BSA (A7906, Sigma, St. Louis, Mo., USA), pH 7.2). Assays were performed using microtiter plates (Costar, Cambridge, Mass., USA).

Peptides and proteins were tested for their ability to activate factor XII. 150 μg ml⁻¹ kaolin, an established activator of factor XII was used as positive control and solvent (H₂O) as negative control. The conversion of Chromozym-PK was recorded kinetically at 37° C. for 60 minutes. Assays were done in duplicates. In control wells, factor XII was omitted from the assay solutions and no conversion of Chromozym-PK was detected. In some assays, albumin was omitted from the reaction mixture. In another type of factor XII activation assay, chromrogenic substrate S-2222 (Chromogenix) was used to follow the activity of factor XII itself. With S-2222, activation of factor XII in plasma was established, using 60% v/v plasma, diluted with substrate and H₂O with or without potential cofactor. Furthermore, auto-activation of factor XII was established by incubating 53 μg/ml purified factor XII in 50 mM Tris-HCl buffer pH 7.5 with 1 mM EDTA and 0.001% v/v Triton-X110, with S-2222 and H₂O with or without potential cofactor.

Binding of tPA, Factor XII and the Fibronectin Type I Domains Thereof to Cross-β Structure-Containing Protein Aggregates

Previously, it was demonstrated that tPA specifically binds to any protein or peptide, as long as ligands have adopted amyloid-like cross-β structure conformation (Kranenburg, 2002). Moreover, binding of tPA to aggregates with cross-β structure is accompanied by activation of tPA. Similar binding and activation characteristics are indicated for factor XII, a serine protease with a similar domain architecture as tPA. Binding of factor XII to protein aggregates with cross-β structure is analyzed in an ELISA set-up. Activation of factor XII by aggregates comprising cross-β structure is analyzed according to the procedure described below. Next, binding of the fibronectin type I domain (F) of tPA and factor XII to amyloid-like aggregates was determined with ELISAs. Aggregates with cross-β structure are immobilized on Exiqon (Vedbaek, Denmark) or Nunc (amino strips, catalogue #076901) Immobilizer plates, or Greiner microlon high-binding plates. Binding of tPA or factor XII was detected with specific antibodies; monoclonal 374b (American diagnostica) for tPA and polyclonal anti-factor XII antibody (Calbiochem). Binding of F domains was determined with F domains comprising a biotin tag, glutathione S transferase tag or His₆-tag. F domains were obtained as described below. In control experiments, binding of K2P-tPA, a tPA analogue that lacks the N-terminal F-EGF-like domain-kringle 1 domain (Reteplase, Boehringer-Ingelheim, Germany), was tested. Binding of tPA and K2P-tPA was tested in the presence of 10 mM ε-amino caproic acid (εACA), a lysine analogue that abolishes the binding of the tPA kringle2 domain to solvent-exposed lysine residues.

### Thioflavin T Fluorescence

Fluorescence of Thioflavin T (ThT) protein/peptide adducts was measured as follows. Solutions of 25 μg/ml of protein or peptide preparations were prepared in 50 mM glycine buffer pH 9.0 with 25 μM ThT. Fluorescence was measured at 485 nm upon excitation at 435 nm. Background signals from buffer, buffer with ThT and protein/peptide solution without ThT were subtracted from corresponding measurements with protein solution incubated with ThT. Regularly, fluorescence of Aβ was used as a positive control, and fluorescence of FP10, a non-amyloid fibrin fragment (Kranenburg, 2002), was used as a negative control. Fluorescence was measured in triplicate on a Hitachi F-4500 fluorescence spectrophotometer (Ltd., Tokyo, Japan).

Thioflavin T fluorescence was determined for human blood platelets that were isolated as described above. The washed platelets in HEPES-Tyrode buffer (200,000/μl) were kept at room temperature. ThT fluorescence was determined at t=0 and at t=72 hours, after storage at room temperature. For the measurements, the platelets were diluted in assay buffer. Appropriate background signal readings were measured and subtracted.

Effects of Protein Aggregates with Cross-β Structure Conformation on the p38MAPK Pathway

Freshly isolated human blood platelets were obtained following the procedure described below. Seventy-five μl of the platelet stock was added to 25 μl of agonist solution and incubated at room temperature for one minute and five minutes. Cells were fixed with 3% v/v formaldehyde and incubated on ice for 15 minutes. Platelets were pelleted upon centrifugation for one minute at 8450*g, and pellets were resuspended in 60 μl reducing sample buffer. After six minutes at 100° C., samples were applied to SDS-PA gels for final Western blot analysis. Blots were first incubated with polyclonal anti-p38MAPK antibody (Cell Signaling Technology) and SWARPO. Then, blots were also incubated with monoclonal anti-actin antibody AC40 (Sigma) and RAMPO for scaling purposes. The relative degree of p38MAPK phosphorylation was determined using densitometric analysis of the blots.

Influence of Protein Aggregates with Cross-β Structure Conformation on Blood Platelet Aggregation

The influence of protein and peptide aggregates with cross-β structure conformation on blood platelet aggregation was tested with washed platelets in an aggregometric assay. Freshly drawn human aspirin-free blood was mixed gently with citrate buffer to avoid coagulation. Blood was spinned for 15 minutes at 150*g at 20° C. and supernatant was collected; platelet-rich plasma (PRP). Buffer with 2.5% trisodium citrate, 1.5% citric acid and 2% glucose, pH 6.5 was added to a final volume ration of 1:10 (buffer-PRP). After spinning down the platelets upon centrifugation for 15 minutes at 330*g at 20° C., the pellet was resuspended in HEPES-Tyrode buffer pH 6.5. Prostacyclin was added to a final concentration of 10 ng/ml, and the solution was centrifuged for 15 minutes at 330*g at 20° C., with a soft brake. The pellet was resuspended in HEPES-Tyrode buffer pH 7.2 in a way that the final platelet number was adjusted to 200,000/μl. Platelets were kept at 37° C. for at least 30 minutes, before use in the assays, to ensure that they were in the resting state.

For the aggregometric assays, 400 μl platelet solution was added to a glass tube with 100 μl containing the agonist of interest, fibrinogen and CaCl₂. Final concentrations of fibrinogen and CaCl₂ were 0.5 mg/ml and 3 mM, respectively. A stirring magnet was added and the apparatus (Whole-blood aggregometer, Chrono-log, Havertown, Pa., USA) was blanked. Aggregation was followed in time by measuring the absorbance of the solution that will decrease in time upon platelet aggregation. As a positive control, 0.5 U/ml thrombin was used. Aggregation was followed for ten minutes.

### Activation of tPA by β2-Glycoprotein I and Binding of Factor XII and tPA to β2-Glycoprotein I

Purification of β2-glycoprotein I (β2gpi) was performed according to established methods. Recombinant human β2gpi was expressed in insect cells and purified as described in de Laat et al. (de Laat, 2005). Plasma-derived 132gpi as used in the factor XII ELISA, the chromogenic plasmin assay and in the anti-phospholipid syndrome antibody ELISA (see below), was purified from fresh human plasma as described in Horbach et al. (Horbach, 1996). Alternatively, β2gpi was purified from either fresh human plasma or from frozen-thawed plasma on an anti-β2gpi antibody affinity column (Horbach, 1998).

Activation of tissue-type plasminogen activator (tPA) (Actilyse, Boehringer-Ingelheim) by β2gpi preparations was tested in a chromogenic plasmin assay (see above). One hundred μg/ml plasma β2gpi or recombinant β2gpi were tested for their stimulatory co-factor activity in the tPA-mediated conversion of plasminogen to plasmin and were compared to the stimulatory activity of cross structure-rich fibrin peptide FP13 (Kranenburg, 2002).

Binding of purified human factor XII from plasma (Calbiochem) or of purified recombinant human tPA to β2gpi purified from human plasma or to recombinant human β2gpi was tested in an ELISA. Ten μg of factor XII or tPA in PBS was coated onto wells of a Costar 2595 ELISA plate and overlayed with concentration series of the two β2gpi preparations. Binding of β2gpi was assessed with monoclonal antibody 2B2 (Horbach, 1998).

From preparations of β2gpi purified from fresh plasma or purified from frozen-thawed plasma, 33 μg was brought onto a 7.5% poly-acrylamide gel. After Western blotting, the nitrocellulose was incubated with 100× diluted anti-human factor XII antibody (Calbiochem) and subsequently 3000× diluted SWARPO (DAKO).

Purified β2gpi from human plasma (400 μg/ml final concentration) was incubated with 100 μM cardiolipin vesicles or with 250 μg/ml DXS500k. Fluorescence of β2gpi in buffer, cardiolipin or DXS500k in buffer, buffer and ThT alone, and of β2gpi-cardiolipin adducts and β2gpi-DXS500k adducts with or without ThT was recorded as described above.

Binding of Anti-β2Gpi Autoantibodies from Antiphospholipid Syndrome Auto-Immune Patients to Immobilized β2Gpi is Inhibited by Recombinant β2Gpi and not by Plasma-Derived β2gpi

When plasma-derived β2gpi is coated onto hydrophilic ELISA plates, anti-β2gpi autoantibodies isolated from plasma of antiphospholipid syndrome auto-immune patients can bind (data kindly provided by B. de Laat, UMC Utrecht). To study the influence of co-incubations of the coated β2gpi with antibodies together with plasma β2gpi or recombinant β2gpi, concentration series of β2gpi were added to the patient antibodies. Subsequently, binding of the antibodies to coated β2gpi was assayed.

### Structural Analysis of Oxidized Low-Density Lipoprotein

Low-density lipoproteins (LDL) were isolated from fresh (<24 hours) human plasma, obtained from the Dutch bloodbank, that was kept at 10° C. LDL was isolated essentially as earlier described. Plasma was centrifuged in an ultracentrifuge for three subsequent cycles. The LDL fraction was isolated and stored under N₂, at 4° C. Before experiments, native LDL (nLDL) was dialyzed overnight at 4° C. against 0.9% w/v NaCl. To obtain oxidized LDL (oxLDL) with varying degrees of oxidation, native was first dialyzed against 0.15 M NaCl solution containing 1 mM NaNO₃, overnight at 4° C. Then, nLDL was diluted to 3-5 mg/ml, and CuSO₄ was added to a final concentration of 25 μM and incubated at 37° C. The degree of oxidation was followed by measurement of diene-formation at λ=234 nm (Ultyrospec 3000 Spectrophotometer (Pharmacia Biotech)). To stop the oxidation reaction, LDL was dialyzed against 0.15 M NaCl, 1 mM NaNO₃ and 1 mM EDTA.

In time, an increase of the oxidation of LDL, as measured by specific diene fluorescence at 243 nm, was completed with Thioflavin T fluorescence and Congo red fluorescence measurements. Fluorescence measurements were performed as described above. In addition, the ability of nLDL and oxLDL to induce tPA activation was tested in the chromogenic plasmin assay. For this purpose, 24% oxidized LDL was used. Finally, the ability of oxLDL to activate factor XII in plasma was tested, as determined by following the conversion of the substrate S-2222, that is cleaved when activated factor XII is present (see above). Activation assays were performed in the wells of 96-well ELISA plates, at 37° C.

### Binding of Amyloid-Specific Dyes to a Fibrin Clot

Pooled human plasma of healthy donors was clotted by adding either phospholipids, CaCl₂ and kaolin when aPTTs are concerned, or tissue factor-rich thromboplastin and CaCl₂ when PT assays are concerned. APTTs and PTs are performed in the presence of concentration series of the amyloid-specific dyes Congo red, Thioflavin S (ThS) or Thioflavin T (ThT), accompanied by the appropriate buffer controls, i.e., Na₂SO₄ for Congo red and NH₄Cl for ThS and ThT. Coagulation velocities under influence of amyloid-specific dyes were measured, or the binding of the dyes was established by visual inspection or by use of direct-light microscopy or fluorescence microscopy.

### Results

### Example 1

### Cross-β Structure is Present in Fibrin and in Synthetic Peptides Derived from Fibrin

It is demonstrated that a fibrin clot stains with Congo red (not shown) and exhibits Thioflavin T fluorescence (FIG. 2, Panel A), indicative of the presence of amyloid structure in a fibrin clot. Using Congo red staining (not shown), circular dichroism measurements and X-ray diffraction analysis, it is shown that synthetic peptides derived from the sequence of fibrin adopt cross-β structure (FIG. 2, Panels B, C). These peptides possess tPA-binding and tPA-activating properties. The presence of cross-β structure in these peptides was found to correlate with the ability to stimulate tPA-mediated plasminogen activation (FIG. 2, Panel D).

In conclusion, these data provide evidence for physiological occurrence/relevance for formation of cross-β structure and the role of this structural element in binding of tPA to fibrin.

### Example 2

### Aβ Contains Cross-β Structure, Binds Plasmin(Ogen) and tPA, Stimulates Plasminogen Activation, Induces Matrix Degradation and Induces Cell Detachment that is Aggravated by Plasminogen and Inhibited by CpB

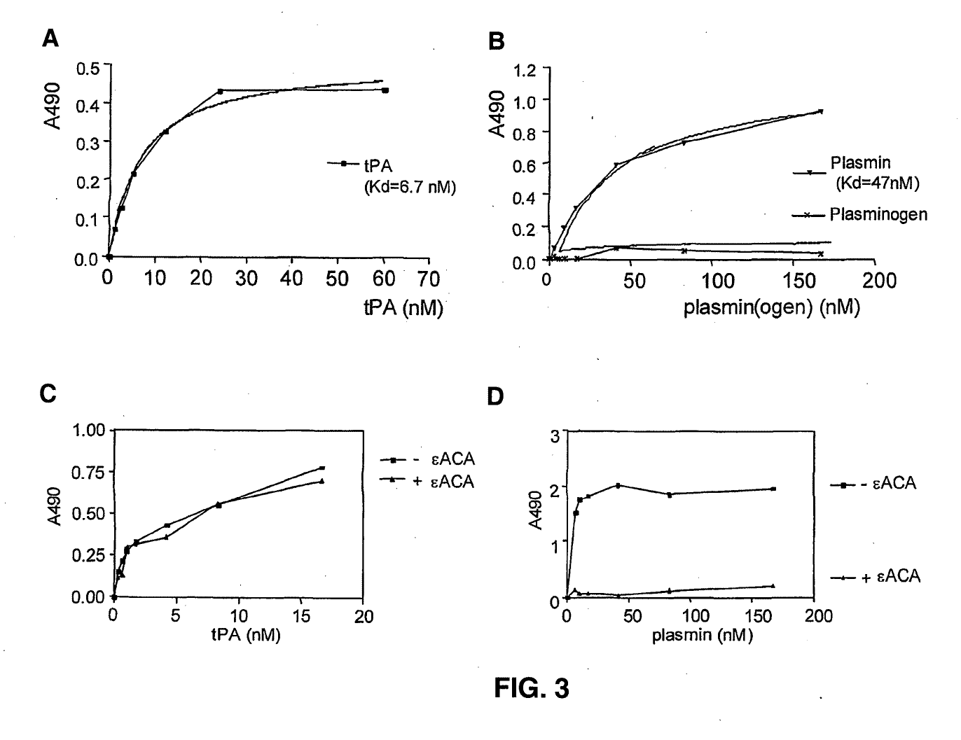

To test whether tPA, plasminogen and plasmin bind Aβ, solid-phase binding assays were performed. Aβ was coated onto plastic 96-well plates and binding of the peptide to either plasmin(ogen) or to tPA was assessed by overlaying the coated peptide with increasing concentrations of either tPA, plasminogen or plasmin. Binding was assessed using specific antibodies to either plasmin(ogen) or to tPA by performing ELISA. FIG. 3, Panel A, shows that tPA binds to Aβ with a Kd of about 7 nM, similar to the Kd of tPA binding to fibrin. In contrast, no detectable binding of plasminogen to Aβ was found (FIG. 3, Panel B). However, activated plasminogen (plasmin) does bind to Aβ, and does so with a Kd of 47 nM. The fact that (active) plasmin, but not (inactive) plasminogen, binds to Aβ, suggests that plasmin activity and, hence, the generation of free lysines, is important for binding of plasmin to Aβ. To test this, use was made of the lysine analogue e-aminocaproic acid (βACA) and tested binding of plasmin and tPA to Aβ in its presence. It is shown that the binding of plasmin to Aβ is completely abolished in the presence of εACA (FIG. 3, Panel D). In contrast, εACA has no effect on the tPA-Aβ interaction (FIG. 3, Panel C). Thus, it is concluded that plasmin binds to free lysines that were generated during the incubation period, presumably via its lysine-binding Kringle domain(s). In line with this, the Kd of plasminogen Kringle domain binding to free lysines in fibrin is similar to the Kd for plasmin binding to Aβ.

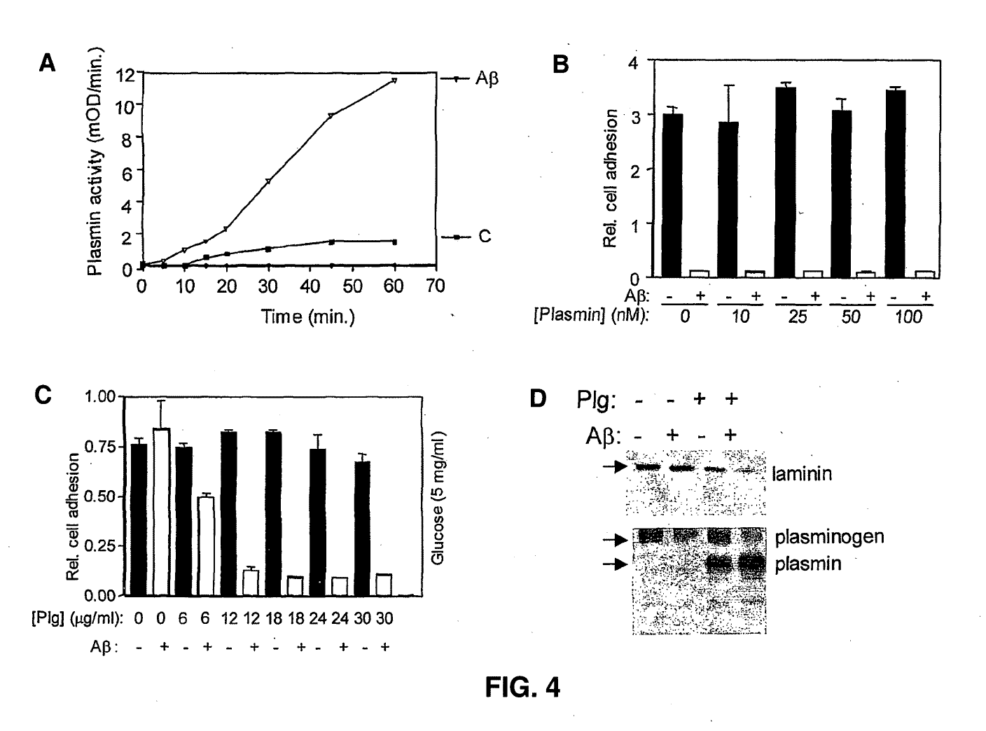

The kinetics of plasminogen activation in the absence and the presence of Aβ was investigated. Aβ potently stimulates the activation of plasminogen by tPA (FIG. 4, Panel A). However, it was found that the reaction proceeds with second-order, rather than with first-order, kinetics. The possibility was considered that the generation of free lysines during the reaction was causing this phenomenon (see below). tPA-mediated plasmin generation has been implicated in neuronal cell death caused by ischemia or by excitotoxic amino acids. Recent data suggest that plasmin can degrade Aβ and thereby prevents Aβ toxicity. It was found that 48 hours following the addition of Aβ to a culture of differentiated N1E-115 cells, the majority of cells had died and detached from the matrix (not shown). When added together with Aβ, plasmin (up to 100 nM) was unable to ameliorate Aβ-induced cell detachment. Even prolonged pre-incubations of Aβ with 100 nM plasmin did not affect Aβ-induced cell detachment (FIG. 4, Panel B). Subsequently, the possibility was considered that plasmin generation may potentiate rather than inhibit Aβ-induced cell detachment and survival. To test this, N1E-115 cells were exposed to suboptimal concentrations of Aβ and low concentrations of plasminogen for 24 hours. In the absence of Aβ, plasminogen has no effect on cell adhesion (FIG. 4, Panel C). However, plasminogen has a dramatic potentiating effect on Aβ-induced cell detachment. The minimal levels of plasminogen that are required to potentiate Aβ-induced cell detachment (10-20 μg/ml) are well below those found in human plasma (250 μg/ml). Plasmin-mediated degradation of the extracellular matrix molecule laminin precedes neuronal detachment and cell death in ischemic brain. Whether Aβ-stimulated plasmin generation leads to laminin degradation was tested. Cell detachment was accompanied by degradation of the extracellular matrix protein laminin (FIG. 4, Panel D).

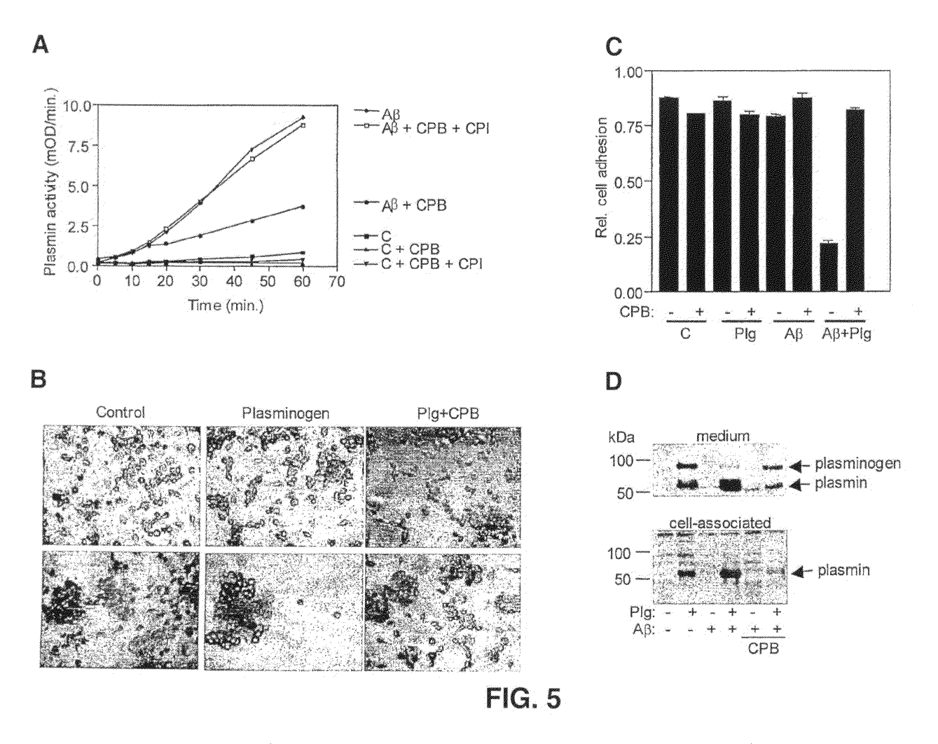

The possibility was considered as to whether the generation of free lysines during AO-stimulated plasmin formation was responsible for the observed second order kinetics. To test this, use was made of carboxypeptidase B (CpB), an enzyme that cleaves of C-terminal lysine and arginine residues) and the CpB-inhibitor CPI. FIG. 5, Panel A, shows that in the presence of CpB, the generation of plasmin is greatly diminished. Furthermore, this effect depends on CpB activity as it is abolished by co-incubation with CPI. FIG. 5, Panel A, also shows that CpB does not completely abolish Aβ-stimulated plasmin generation, but that the reaction proceeds with slow first-order kinetics. These data suggest that the (plasmin-mediated) generation of free lysines during the reaction is essential for efficient Aβ-stimulated plasmin generation, presumably by supporting plasminogen and tPA binding through interaction with their respective Kringle domains. A similar dependency on the generation of C-terminal lysines has been shown for efficient fibrin-mediated plasmin generation.⁵⁸ These results show that Aβ-stimulated plasmin formation is regulated by carboxypeptidase B in vitro. Thus, if cell detachment is the result of plasmin generation, CpB may affect Aβ-induced cell detachment and/or viability. It is shown that cell detachment induced by plasminogen and Aβ is completely prevented by co-incubation with CpB (FIG. 5, Panels B and C). This is accompanied by a complete inhibition of Aβ-stimulated plasmin formation, both in the medium and on the cells (FIG. 5, Panel D).

### Example 3

### Endostatin can Form Amyloid Fibrils Comprising Cross-β Structure

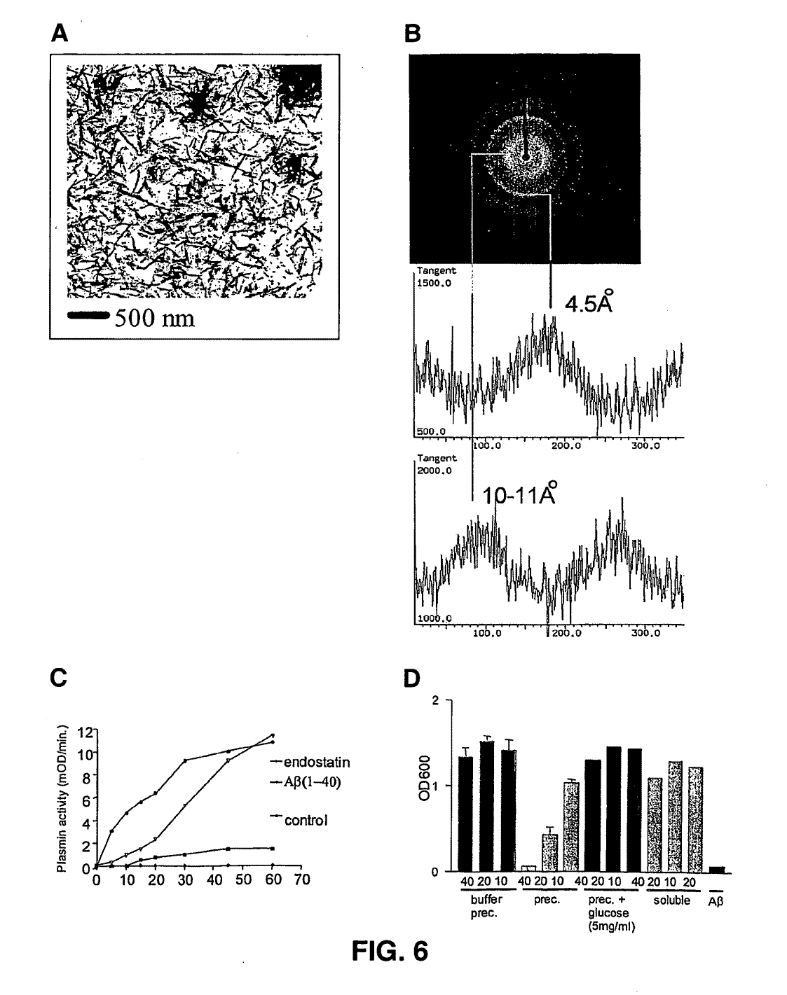

Using Congo red staining (not shown), X-ray diffraction analysis and TEM, the presence of cross-β structure in aggregated endostatin from Escherichia coli, as well as from Pichia pastoris, and the ability of endostatin to form amyloid fibrils is demonstrated (FIG. 6, Panels A and B). Bacterial endostatin produced reflection lines at 4.7 Å (hydrogen-bond distance), as well as at 10-11 Å (inter-sheet distance). The reflection lines show maximal intensities at opposite diffraction angles. The fiber axis with its 4.7 Å hydrogen bond repeat distance is oriented along the vertical capillary axis. This implies that inter-sheet distance of 10-11 Å is perpendicular to these hydrogen bonds. This is consistent with the protein being a cross-β sheet conformation with a cross-β structure. Intramolecular sheets in a globular protein cannot cause a diffraction pattern that is ordered in this way. From the amount of background scattering, it follows that only part of the protein takes part in cross-β structure formation. It was found that the presence of cross-β structures in endostatin correlates with its ability to stimulate tPA-mediated plasminogen activation (FIG. 6, Panel C) and correlates with neuronal cell death (FIG. 6, Panel D).

It is demonstrated herein that endostatin is an example of a denatured protein that is able to stimulate the suggested cross-β pathway.

### Example 4

### IAPP Binds tPA and Stimulates tPA-Mediated Plasminogen Activation

Amyloid deposits of IAPP are formed in the pancreas of type II diabetic patients. IAPP can cause cell death in vitro and is, therefore, thought to contribute to destruction of β-cells that is seen in vivo, which leads to insufficient insulin production. IAPP forms fibrils comprising cross-β structure.

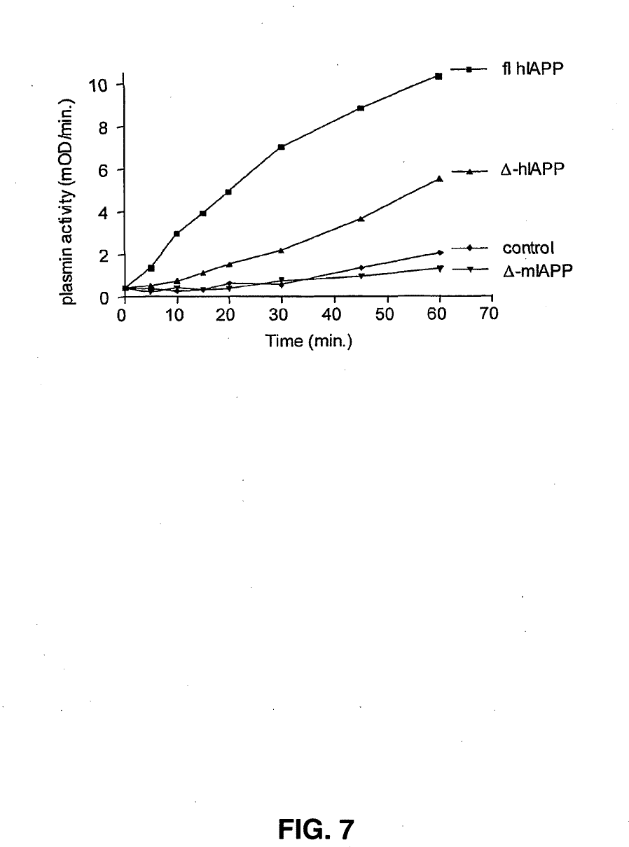

Whether IAPP could stimulate tPA-mediated plasminogen activation was tested (FIG. 7). Indeed, similar to Aβ, IAPP can enhance the formation of plasmin by tPA.

### Example 5

### Glycated Albumin Binds. Thioflavin T and tPA, and Aggregates as Amyloid Fibrils Comprising Cross-β Structure

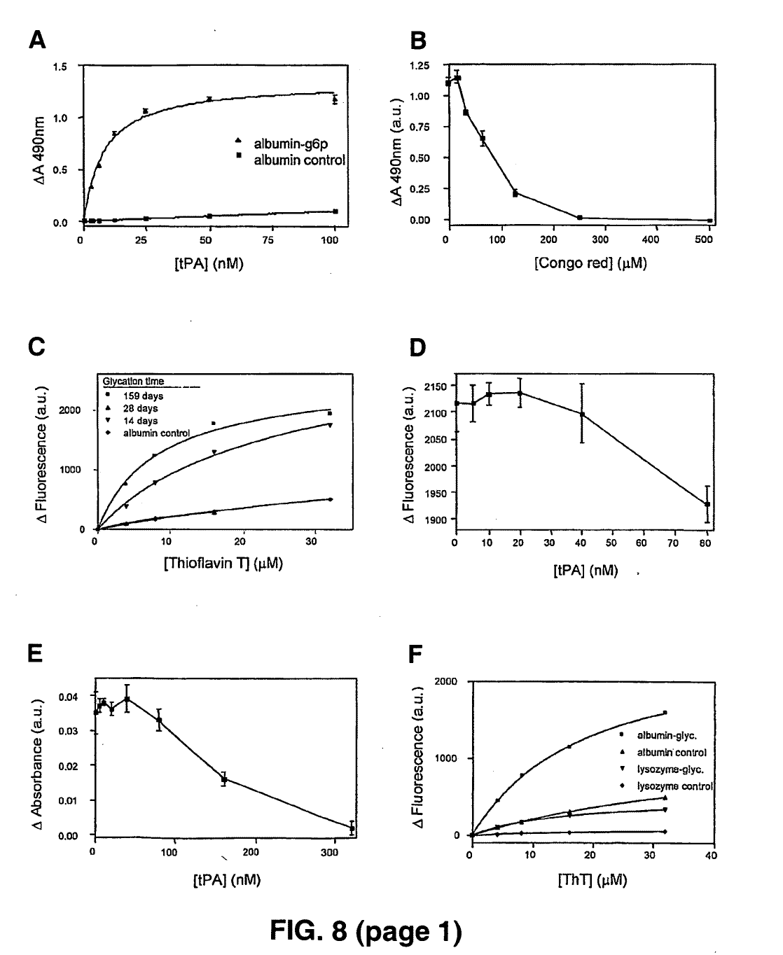

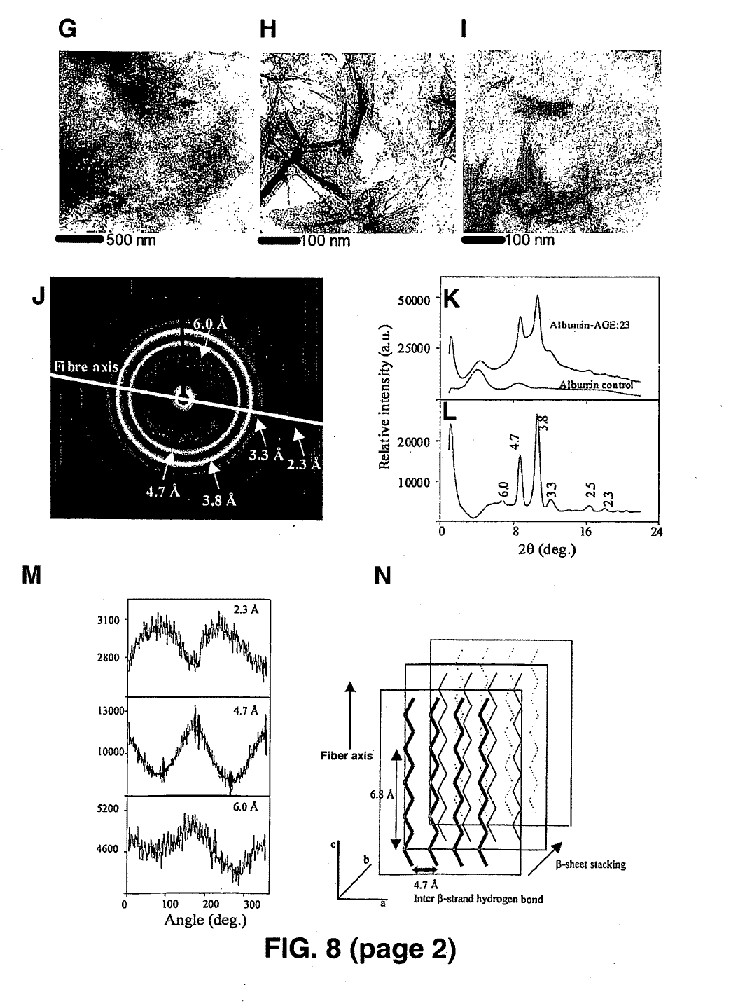

It has been demonstrated that glycation of several proteins can induce or increase the ability of these proteins to bind tPA and stimulate tPA-mediated plasmin formation. It is shown herein that glycation of albumin with g6p not only confers high-affinity tPA binding to albumin (FIG. 8, Panel A), but also leads to its ability to bind Thioflavin T (FIG. 8, Panel C). Binding of tPA can be competed with Congo red (FIG. 8, Panel B). In addition, binding of Thioflavin T to glycated albumin can be competed by tPA (FIG. 8, Panels D, E). The fact that Congo red and/or Thioflavin T and tPA compete, illustrates that they have either the same or overlapping binding sites.

Analyses with TEM of the g6p-modified albumin preparations revealed that after a four-week incubation, amorphous albumin aggregates are formed (FIG. 8, Panel G), which exhibits a CD spectrum indicative for the presence of 7% of the albumin amino acid residues in β-sheet (Table 1). Prolonged incubation (up to 23 weeks) resulted in a switch to highly ordered sheet-like fibrous structures, with a length of approximately 500 nm and a diameter ranging from about 50 to 100 nm FIG. 8, Panel H). These fibers showed an increase to 19% β-sheet, when analyzed with CD spectropolarimetry (Table 1). Albumin from a different batch that was glycated in the same way, already showed bundles of fibrous aggregates after a two-week period of incubation (FIG. 8, Panel I), whereas an increase in β-sheet content is not detected with CD spectropolarimetry (Table 1). In each bundle, about ten separate linear 3-S-nm-wide fibers with a length of 200-300 nm can be identified. On top of each bundle, regularly distributed spots are seen, with a diameter of approximately 5 nm. These spots may be accounted for by globular albumin molecules that are bound to the fibers, or alternatively, that are partly incorporated in the fibers. Aggregates were absent in control albumin (not shown) and no β-sheets were measured using CD spectropolarimetry (Table 1). The fibrous structures obtained after two-week and 23-week periods of glycation enhance the fluorescence of Thioflavin T (ThT) in a similar way, whereas the amorphous precipitates obtained after four weeks hardly increased the fluorescent signal.

X-ray fiber diffraction analyses revealed that albumin-g6p (23 weeks) comprises a significant amount of crystalline fibers (FIG. 8, Panels J, L), whereas diffraction patterns of albumin-g6p (two weeks) and albumin-g6p (four weeks) show features originating from amorphous precipitated globular protein, very similar to the patterns obtained for albumin controls (FIG. 8, Panel K). For albumin-g6p (23 weeks), the 4.7 Å repeat corresponds to the characteristic hydrogen-bond distance between β-strands in β-sheets. The 2.3 and 3.3 Å repeats have a preferred orientation perpendicular to the 4.7 Å repeat (FIG. 8, Panel M). Combining the 2.3 and 3.3 Å repeats suggests that the fiber axis is oriented perpendicular to the direction of the hydrogen bonds, with a repeat of 6.8 Å. This dimension corresponds to the length of two peptide bonds and indicates that β-strands run parallel to the fiber axis. This implies that the albumin-g6p (23 weeks) structure is composed of cross-β structure consisting of packed β-sheets of hydrogen-bonded chains (FIG. 8, Panel N). A similar orientation is found in amyloid fibrils of the first predicted α-helical region of PrP^{c}. When the a-axis is 9.4 Å, or alternatively 4.7 Å, and the c-axis is 6.8 Å, the 2.5 and 6.0 Å repeats can only be indexed as (h k 1). This implies a highly ordered b-axis repeat, corresponding to the inter β-sheet distance. With a-axis and c-axis of 4.7, or 9.4 Å and 6.8 Å, respectively, the strong 3.8 Å repeat should be indexed as (2 0 1) or (1 0 1). Considering all observations, it is clear that the albumin-g6p fibers (23 weeks) are built up by cross-β structures, a characteristic feature of amyloid fibrils.

These results show that due to incubation and/or modification with sugar moieties, cross-β structures in albumin are formed that are able to support tPA binding.

### Example 6

### Glycation of Hemoglobin Induces tPA Binding and Fibril Formation

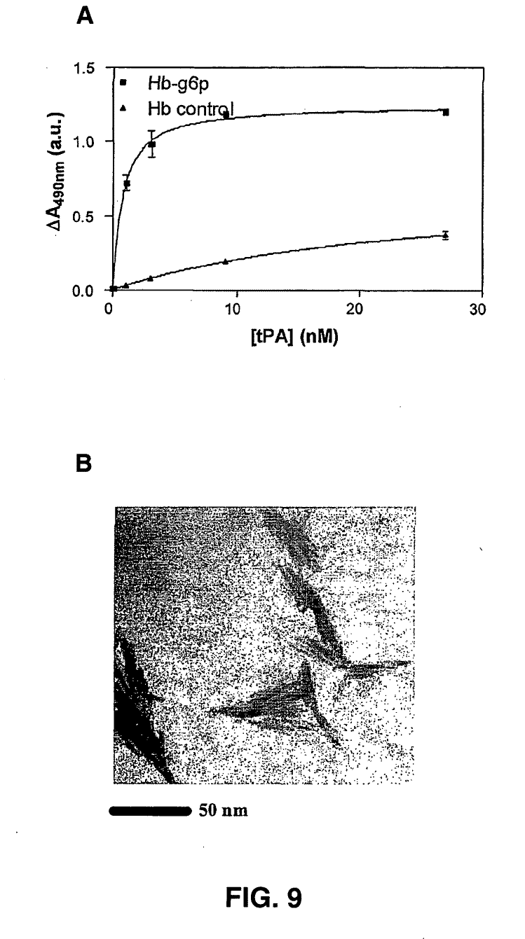

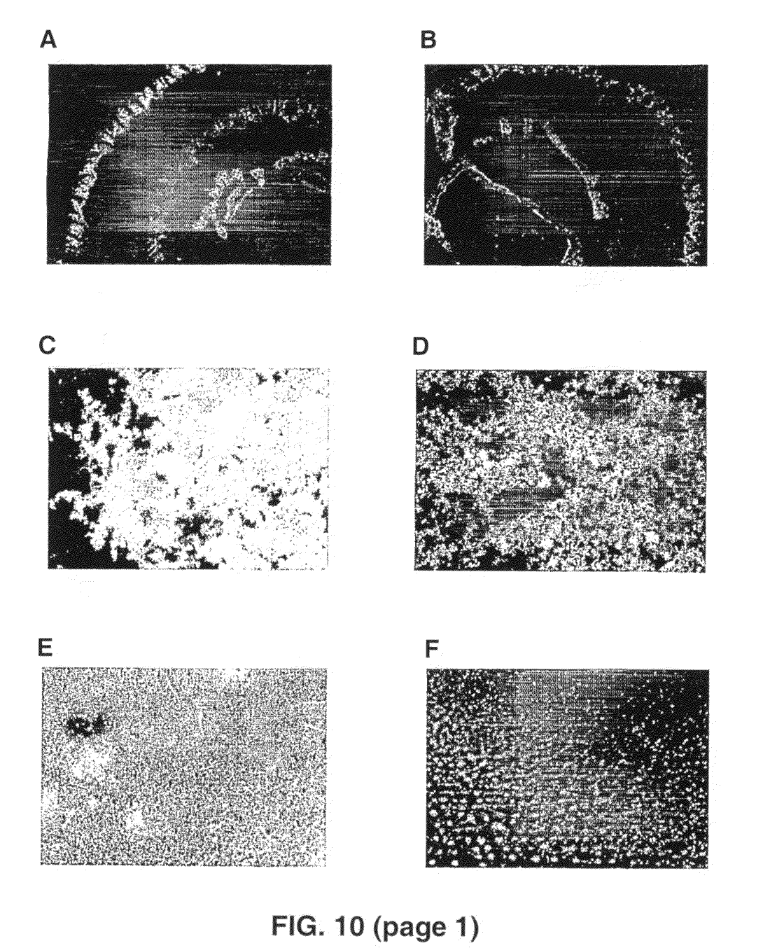

Incubation of human hemoglobin with g6p resulted in high-affinity tPA binding (FIG. 9, Panel A). Amorphous aggregated Hb-g6p adducts including fibrils were observed with TEM (FIG. 9, Panel B), whereas control Hb did not aggregate (not shown). Human Hb of diabetes mellitus patients has the tendency to form fibrillar aggregates, once more than 12.4% of the Hb is glycated (Table 2).

### Example 7

### Amyloid Albumin is Formed Irrespective of the Original Carbohydrate (Derivative)

From the above listed observations, it is clear that modification of —NH₂ groups of albumin with g6p induces formation of amyloid cross-β structure. The next question addressed was whether triggering of refolding of globular albumin into an amyloid fold was a restricted property of g6p, or whether amyloid formation occurs irrespective of the original carbohydrate or carbohydrate derivative used for AGE formation. Albumin solutions were incubated for 86 weeks at 37° C. with 1 M g6p, 250 mM DL-glyceraldehyde/100 mM NaCNBH₃, 1 M β-D-(−)-fructose, 1 M D(+)-glucose, 500 mM glyoxylic acid/100 mM NaCNBH₃, and corresponding PBS and PBS/NaCNBH₃ buffer controls. Glyceraldehyde and glyoxylic acid are carbohydrate derivatives that are precursors of AGE in Maillard reactions. After 86 weeks, albumin-glyceraldehyde and albumin-fructose were light-yellow/brown suspensions. Controls were colorless and clear solutions. Albumin-glucose and albumin-glyoxylic acid were clear light-yellow to light-brown solutions. Albumin-g6p:86 was a clear and dark brown solution. AGE formation was confirmed by autofluorescence measurements using AGE-specific excitation/emission wavelengths (not shown), binding of moab anti-AGE 4B5 (not shown) and binding of poab anti-AGE (not shown). As expected, albumin-glyoxylic acid did not show an autofluorescent signal due to the fact that (mainly) non-fluorescent carboxymethyl-lysine (CML) adducts are formed.

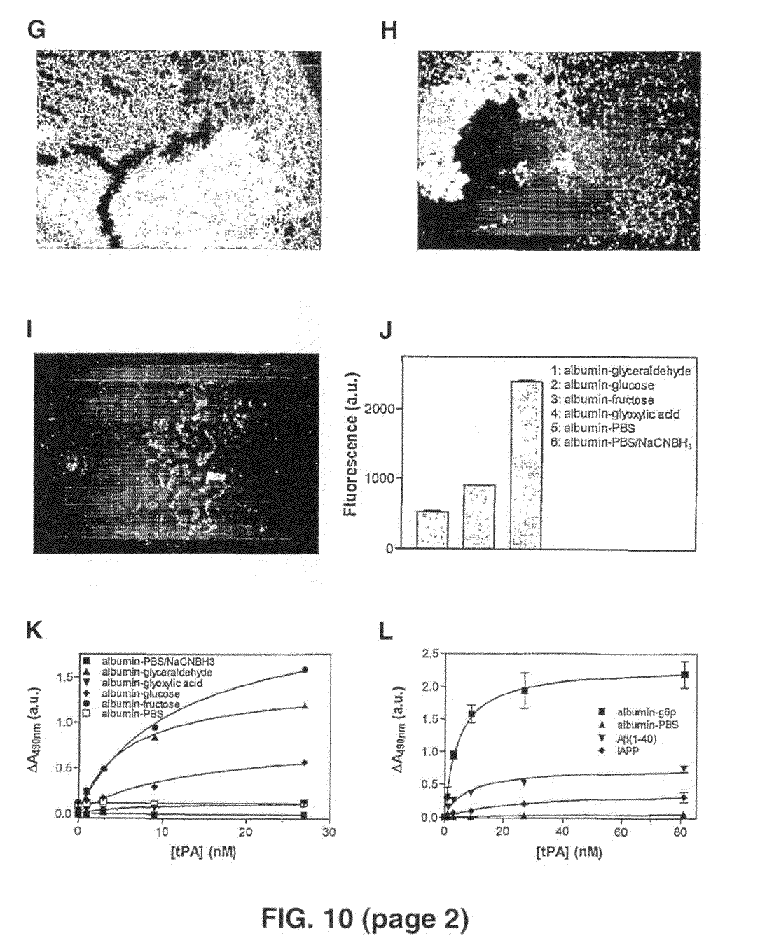

The autofluorescence data and the binding of AGE-specific antibodies listed above show that various carbohydrates and carbohydrate derivatives can lead to similar AGE structures. Using g6p as starting point for AGE formation, it was shown that albumin adopted amyloid properties, similar to those observed in well-studied fibrils of Aβ and IAPP. Therefore, testing for the presence of amyloid structures in the albumin-AGE adducts obtained with alternative carbohydrates and derivatives was performed. Measurements were taken for fluorescence of albumin-AGE-ThT solutions (FIG. 10, Panel J) and of air-dried albumin-AGE preparations that were incubated with Congo red (FIG. 10, Panels A-I). Incubation of albumin with glyceraldehyde, glucose or fructose resulted in an increased fluorescent signal of ThT (FIG. 10, Panel J). After subtraction of background signals of ThT and buffer, no specific amyloid—ThT fluorescence was measured for albumin-glyoxylic acid and buffer controls. Albumin-g6p, albumin-glyceraldehyde and albumin-fructose gave a Congo red fluorescent signal similar to signals of Aβ and IAPP (FIG. 10, Panels C-E,G-H). With albumin-glucose, a uniformly distributed pattern of fluorescent precipitates is observed (FIG. 10, Panel F). With albumin-glyoxylic acid and buffer controls, hardly any staining is observed (FIG. 10, Panels A-B, I). These ThT and Congo red fluorescence data show that, in addition to albumin-g6p, albumin-glyceraldehyde, albumin-glucose and albumin-fructose have amyloid-like properties. To further substantiate these findings, testing for binding of amyloid-specific serine protease tPA in an ELISA was performed. The enzyme bound specifically to albumin-g6p, albumin-glyceraldehyde, albumin-glucose and albumin-fructose (FIG. 10, Panels K-L) and to positive controls Aβ and IAPP, as was shown before (Kranenburg, 2002). No tPA binding is observed for albumin-glyoxylic acid and buffer controls.

From the ThT, Congo red and tPA data, it is clear that inducing amyloid properties in albumin is not an exclusive property of g6p. Apparently, a spectrum of carbohydrates and carbohydrate derivatives, comprising g6p, glucose, fructose, glyceraldehyde, and likely more, has the capacity to trigger the switch from a globular native fold to the amyloid cross-β structure fold, upon their covalent binding to albumin.

### Example 8

### Analysis of Congo Red Binding and tPA Binding to Aβ

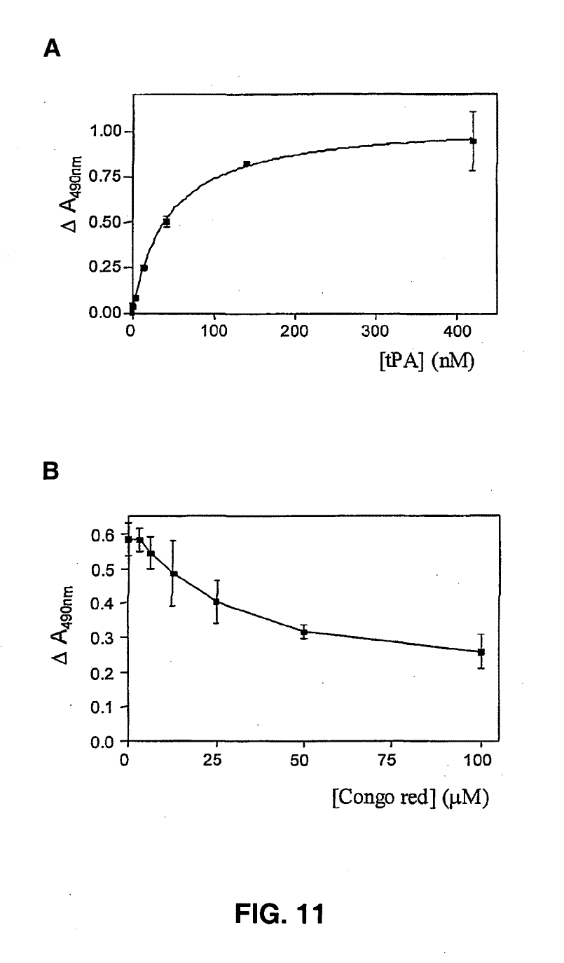

It has been demonstrated that Aβ can bind tPA and Congo red. It is shown that the binding of tPA to Aβ can be competed by Congo red (FIG. 11). These results support the finding that structures in Aβ, fibrin and glycated albumin are similar and are able to mediate the binding to tPA.

### Example 9

### Binding of Human FXII to Amyloid Peptides and Proteins that Contain the Cross-β Structure Fold

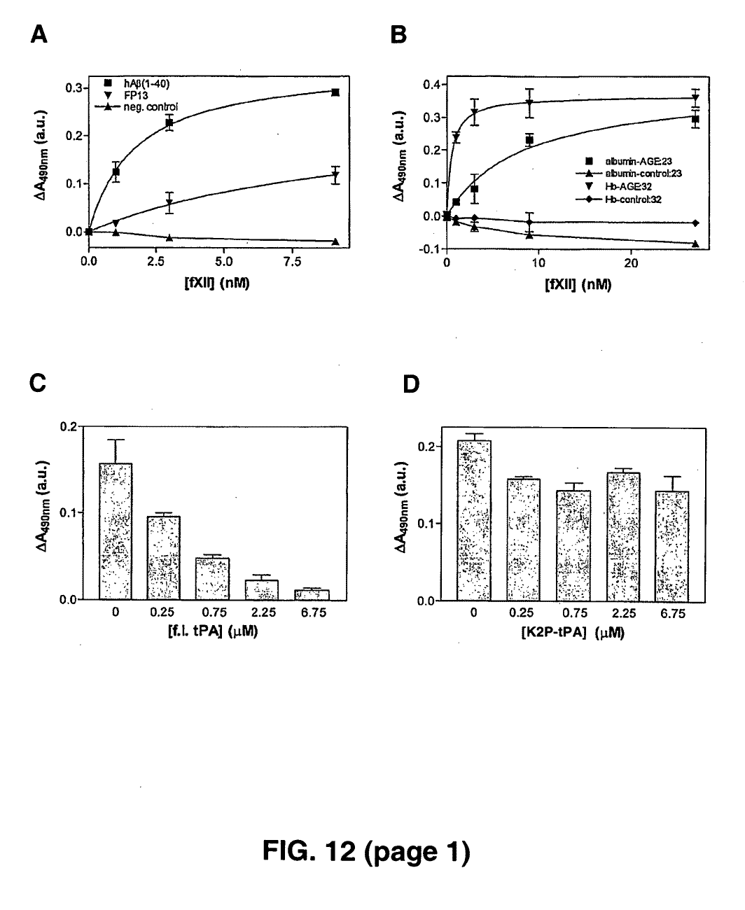

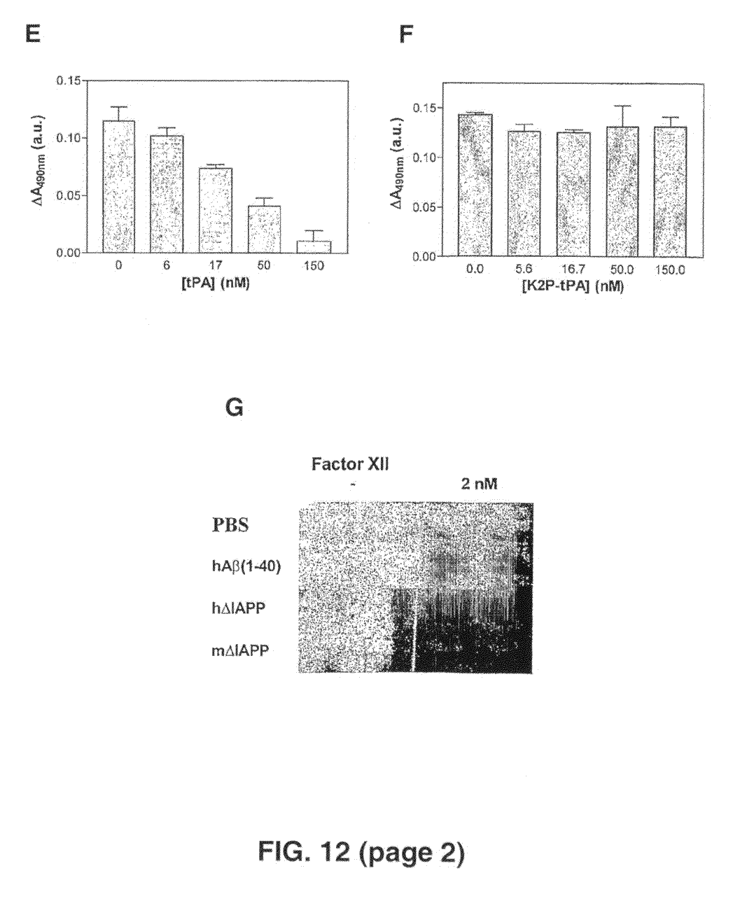

The graphs in FIG. 12 show that FXII binds specifically to all amyloid compounds tested. k_{D}s for hAβ(1-40), FP13, albumin-AGE and Hb-AGE are approximately 2, 11, 8 and 0.5 nM, respectively. The data obtained with the competitive FXII-tPA ELISA show that tPA efficiently inhibits binding of FXII to amyloid (poly)peptides (FIG. 12). From these data, it is concluded that FXII and full-length tPA compete for overlapping binding sites on hAβ(1-40). K2P-tPA does not inhibit FXII binding. Binding of FXII to albumin-AGE is also effectively abolished by tPA but not by K2P-tPA, similar to what was observed for hAβ(1-40). This indicates that FXII may bind in a similar manner to hAβ(1-40) and albumin-AGE. In addition, these data show that the first three domains of tPA (finger, EGF-like, kringle 1) seem to be involved in the inhibitory effect of full-length tPA on interactions between FXII and amyloid hAβ(1-40) or albumin-AGE. Using a dot-blot assay, binding of FXII to spotted amyloid hΔIAPP and hAβ(1-40) was tested. No binding of FXII was observed for negative controls PBS and MΔIAPP (FIG. 12). However, FXII specifically bound to hAβ(1-40), as well as to hΔIAPP (FIG. 12). These data, together with the ELISA data shown in FIG. 12, Panels A-F, suggest that FXII can bind to polypeptides that do not share amino acid sequence homology, though which share the cross-β structure fold. This is in accordance with recent data on interactions between tPA and polypeptides that contain the amyloid-specific fold.

### Example 10

### Binding of tPA to the Cross-β Structure-Containing Molecules, Aβ and Glycated Albumin Requires the Presence of an N-Terminal Region in tPA, which Contains the Finger Domain

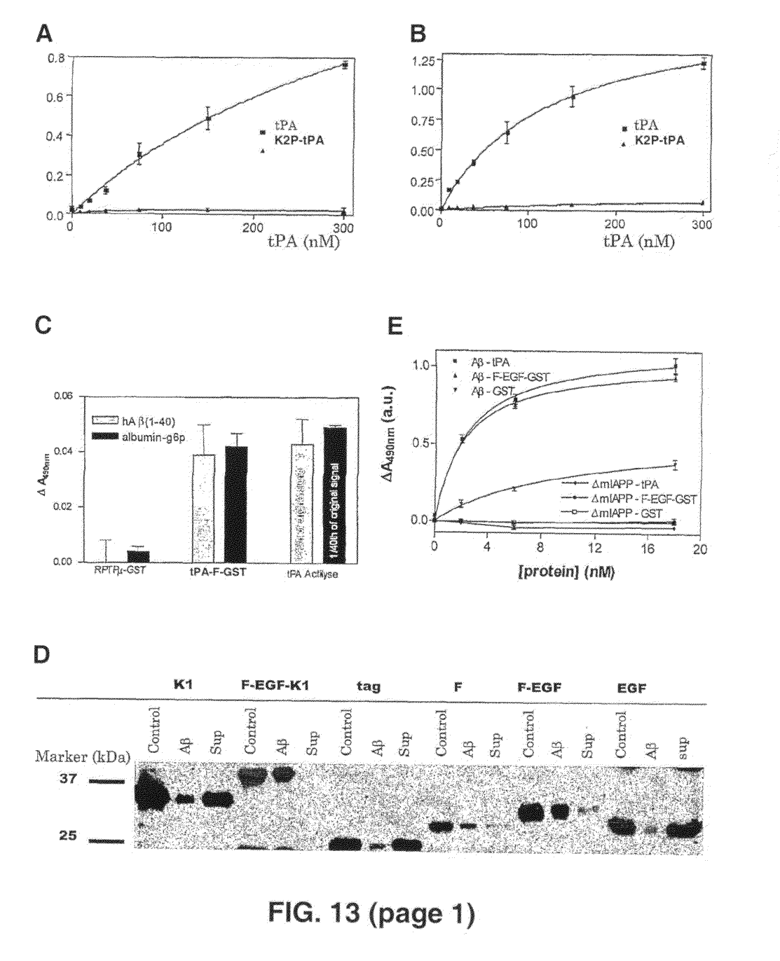

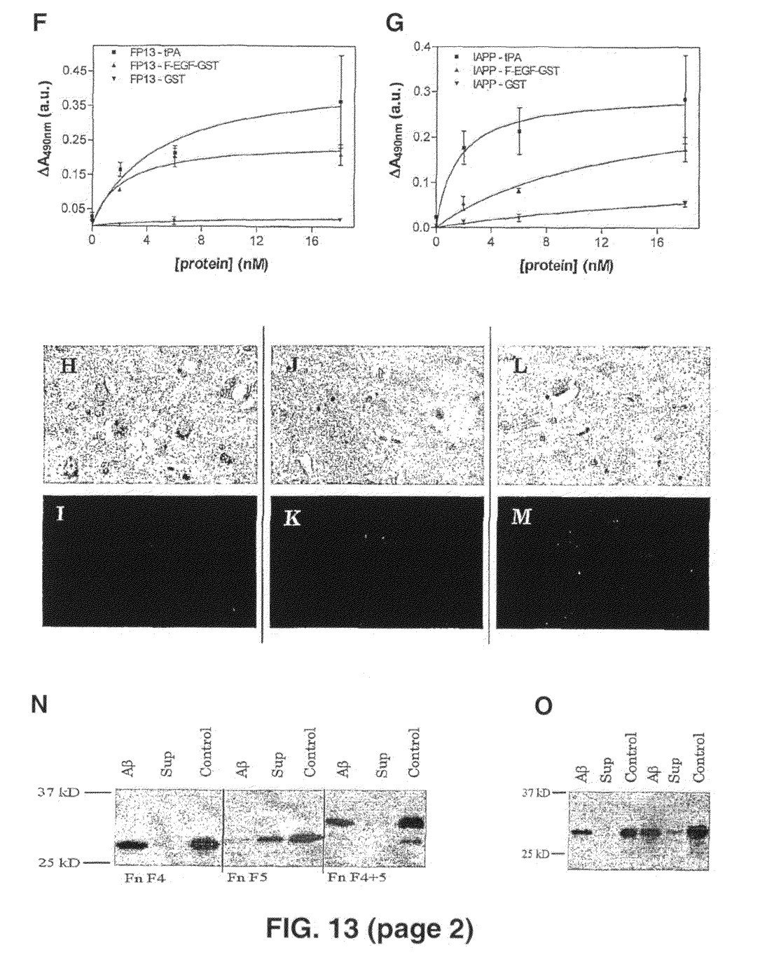

Several domains in tPA have been assigned to mediate binding to fibrin or fibrin fragments. However, it is unknown which domain of tPA is needed for binding to Aβ or other cross-β structure-containing molecules. It is shown that a mutated tPA, termed reteplase, which lacks the N-terminal finger, EGF and kringle 1 domain (K2-tPA) is unable to bind cross-β structure-comprising molecules (FIG. 13, Panels A, B). These results suggest that the N-terminal region is required for binding of tPA to fibrils comprising cross-β structure.

Expression and Purification of tPA-F-GST and RPTP-GST

Purification of the GST-tagged constructs tPA-F-GST and RPTPμ-GST(control) from 293T medium using glutathione coupled to Sepharose 4B beads resulted in single bands of approximately 35 kDa and 150 kDa, respectively (not, shown). Traces of BSA, originating from the FCS used in the medium, were also present.

ELISA: Binding of tPA-F-GST and RPTP-GST to Human Aβ(1-40) and Glycated Albumin

In the ELISA, control tPA bound to both human Aβ(1-40) and albumin-g6p in the presence of excess εACA (FIG. 13, Panel C). This shows that in the assay used, tPA is capable of binding to fibrous amyloids, in a kringle2-independent manner. The tPA-F domain bound to human Aβ(1-40) and to albumin-g6p, whereas no binding was observed with RPTPμ-GST. Therefore, binding observed with tPA-F-GST is specific and does not originate from the GST tag. This result points to the tPA finger domain as a specific domain designed by nature for binding to cross-β structured amyloid fibrils.

cDNA constructs in pcDNA3 of the F, F-EGF, EGF, F-EGF-K1 and K1 fragments of human tPA were prepared. Recombinant proteins with a C-terminal GST tag were expressed in BHK cells and secreted to the medium. Medium from BHK cells expressing the GST tag alone was used as a control. Conditioned medium was used for pull-down assays using Aβ and IAPP fibrils, followed by Western blot analyses. Efficient binding to Aβ is evident for all three tPA mutants that contain the finger domain, i.e., F-GST, F-EGF-GST and F-EGF-K1-GST (FIG. 13D). The K1-GST and EGF-GST constructs, as well as the GST tag alone, remain in the supernatant after Aβ incubation. A similar pattern was obtained after IAPP pull-downs (not shown).

Binding was compared of purified tPA-F-EGF-GST, recombinant full-length Actilyse tPA and a GST control to immobilized amyloid Aβ, amyloid fibrin fragment α₁₄₈-160 FP13, amyloid IAPP and to non-amyloid mΔIAPP control (FIG. 13, Panels E-G). Full-length tPA and tPA-F-EGF-GST bind to all three amyloid peptides; for AD, k_{D}s for tPA and F-EGF are 2 and 2 nM, respectively, for FP13 5 and 2 nM, for IAPP 2 and 13 nM. No binding to non-amyloid mΔIAPP is observed (FIG. 13, Panel E). GST does not bind to FP13 and IAPP, while some binding is detected to Aβ. This may reflect the small fraction of GST that bound to Aβ in the pull-down assay (FIG. 13, Panel D).

With immunohistochemical analysis, binding of the purified recombinant tPA-F-EGF-GST construct to paraffin sections of human brain afflicted with AD was tested. Presence of amyloid depositions was confirmed by the Dept. of Pathology (UMC Utrecht) using standard techniques. In the experiments, these amyloid depositions were located using Congo red fluorescence (FIG. 13, Panels I, K, M). In FIG. 13, Panels H-K, it is clearly seen that areas that are positive for Congo red binding coincides with areas that are positive for tPA-F-EGF-GST binding. Control stain with GST does not show specific binding of the tag alone (FIG. 13, Panels L-M).

At present, based on sequential and structural homology, next to tPA three proteins are known that contain one or more finger domains, i.e., HGFa (one F domain), FXII (one F domain), Fn (one stretch of six F domains, two stretches of three F domains). From the above-listed results, it is concluded that the F domain of tPA plays a crucial role in binding of tPA to amyloid (poly)peptides. It is hypothesized that the finger domain could be a general cross-β structure-binding module. Presently, four proteins, tPA, FXII, HGFa and fibronectin, are known that contain a finger motif. FIG. 14, Panel A, schematically depicts the localization of the finger module in the respective proteins. FIG. 14, Panel B shows an alignment of the human amino acid sequences of the finger domains in these four proteins. FIG. 14, Panel C shows a schematic representation of the three-dimensional structure of the finger domain of tPA, and of the fourth and fifth finger domains of fibronectin. To test the hypothesis that finger domains, in general, bind amyloid, the F domains of HGFa and FXII were cloned, as well as the fourth and fifth F domains of Fn, which are known for their capacity to bind to fibrin. Using a pull-down assay, it was shown that Fn F4-GST and Fn F4-S-GST, as well as FXII F-GST and HGFa-F-GST, specifically bind to Aβ (FIG. 13, Panels M-N) and IAPP (not shown). Fn F5-GST binds to Aβ to some extent, however, it is extracted less efficiently from the medium and seems to be partly released during the washing procedure of the amyloid pellet (FIG. 13, Panel M). No construct was left in the medium after extraction of positive control tPA-F-EGF-GST, whereas no negative control GST was detected in the pellet fraction (not shown). These data show that binding to amyloid (poly)peptides is not a unique capacity of the tPA-F domain, yet a more general property of the F domains tested. Moreover, these data indicate that observed binding of FXII to amyloid (poly)peptides, as shown in FIG. 13, Panels A, H, is regulated via the F domain.

### Example 11

### Amyloid-Binding Domain of tPA

The finger domain of tPA has been shown to be of importance for high-affinity binding to fibrin. The present results using Reteplase (K2P-tPA) and F-tPA, F-EGF-tPA and F-EGF-K1-tPA indicate an important role for the N-terminal finger domain of tPA in binding to stimulatory factors other than fibrin. Thus far, all these factors bind Congo red and contain cross-β structure. Furthermore, the binding site of fibronectin for fibrin has been mapped to the finger domain tandem F4-F5. It has been demonstrated that plasminogen activation by full-length tPA, in the presence of fibrin fragment FCB2, can be inhibited by fibronectin. Taken together, these observations suggest that tPA and fibronectin compete, via their finger domain, for the same or overlapping binding sites on fibrin. The data now show that the F4-5 domains of Fn bind to amyloid Aβ.

### Example 12

### Binding of Anti-AGE Antibodies to Amyloid (Poly)Peptides and Binding of Anti-Aβ to Protein-AGE Adducts

Recently, O'Nuallain and Wetzel (O'Nuallain, 2002) showed that antibodies elicited against a peptide with amyloid characteristics, can bind to any other peptide with similar amyloid properties, irrespective of amino acid sequence. Based on these data and on the observations that tissue-type plasminogen activator and factor XII can bind to a family of sequence-unrelated polypeptides that share the amyloid-specific cross-β structure fold, it was hypothesized that a broader class of proteins can display affinity towards this structural unit, rather than towards a linear or conformational epitope, built up by specific amino acid residues. This hypothesis prompted the question as to whether antibodies elicited against albumin-AGE that contains the amyloid cross-β structure fold, also display the broad-range specificity towards any (poly)peptide that bears this cross-β structure fold.

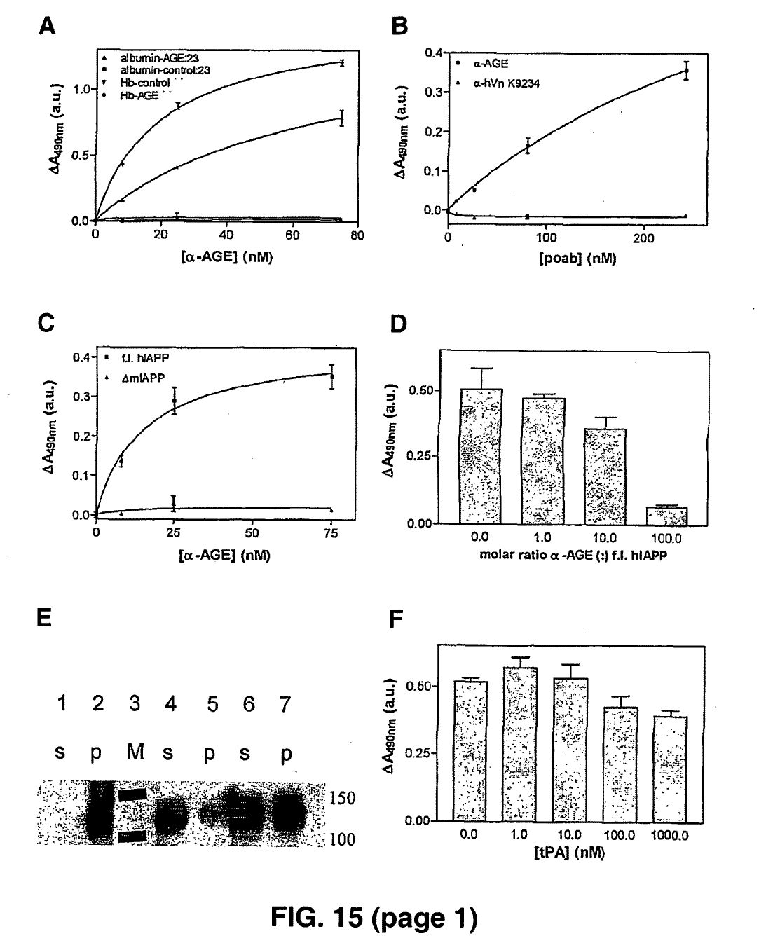

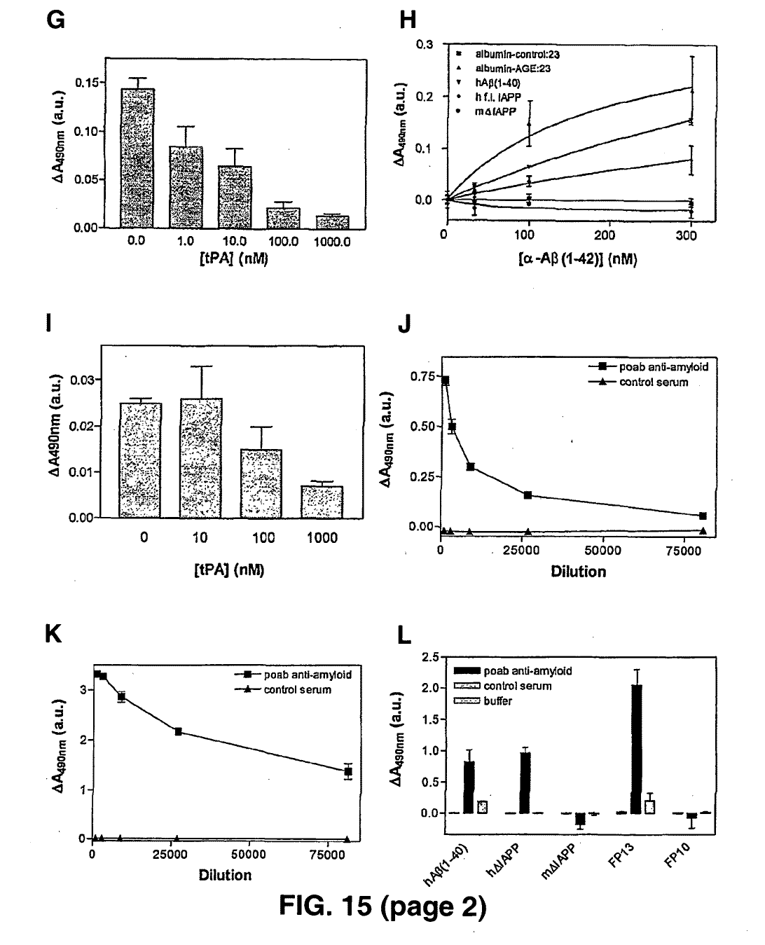

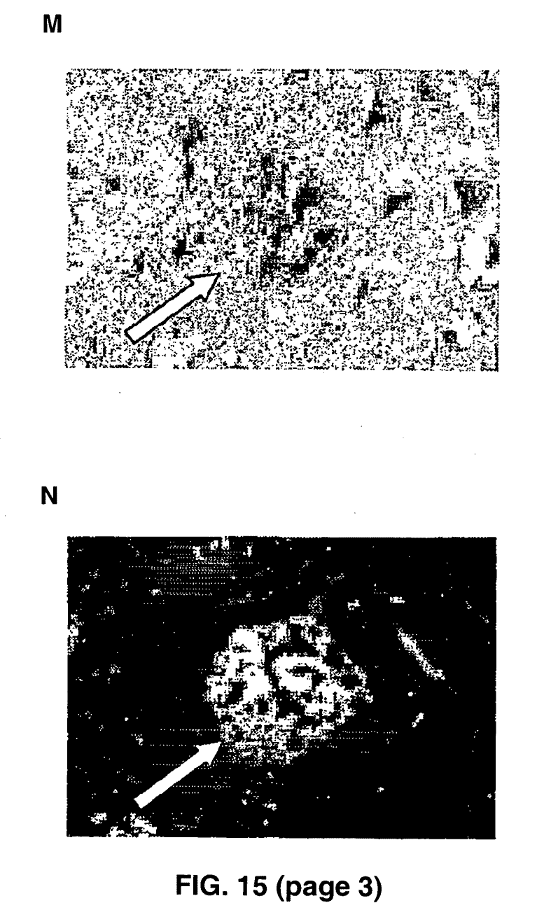

In an ELISA set-up, β-AGE1, which was elicited against g6p-glycated albumin-AGE, binds to amyloid albumin-AGE:23 (K_{d}=66 nM) and Hb-AGE:32 (K_{d}=20 nM), as well as to Aβ(1-40) (K_{d}=481 nM) and IAPP (K_{d}=18 nM) (FIG. 15, Panels A-C). Negative controls were non-glycated albumin and Hb, non-amyloid peptide mouse ΔIAPP for IAPP and polyclonal anti-human vitronectin antibody α-hVn K9234 for Aβ. To test whether the same fraction of α-AGE1 binds to IAPP and Aβ, the antibody was pre-incubated with LAPP fibrils, followed by pelleting of the fibrils, together with the possible amyloid-binding fraction of α-AGE1. Binding of α-AGE1, left in the supernatant, to Aβ(1-40) was reduced (FIG. 15, Panel D). This indicates that the same fraction of α-AGE1 binds to IAPP and Aβ(1-40). With a pull-down assay, the binding of anti-AGE1 to amyloid peptides in an alternative way was assessed. After incubation of anti-AGE1 solutions with amyloid fibrils Aβ(16-22) (FIG. 15, Panel E; lane 1-2), Aβ(1-40) FIG. 15, Panel E; lane 4-5) and IAPP (FIG. 15, Panel E, lane 6-7), and subsequent pelleting of the amyloid fibrils, the supernatant was completely depleted from α-AGE1 by Aβ(16-22). With IAPP, approximately 50% of the antibody is found in the amyloid fraction, whereas less antibody is pelleted with Aβ(1-40). These data obtained in a complementary way again show that anti-AGE1 can bind to amyloid peptides, which share no amino acid sequence homology with albumin-AGE:23, though which share the cross-β structure fold. In addition, the observation that binding of tPA to amyloid peptides inhibits binding of anti-AGE1, also indicates that anti-AGE1, like tPA, binds to the cross-β structure fold (FIG. 15, Panels F-G). The observation that tPA reduces anti-AGE1 binding to Aβ to a lesser extent than the reduction seen with IAPP, is putatively related to the higher number of anti-AGE1 binding sites on coated Aβ, when compared with IAPP (see FIG. 15, Panels B-C), and to the higher affinity of tPA for IAPP (k_{D}=6 nM) than for Aβ (k_{D}=46 nM), when using Exiqon ELISA plates (not shown). The binding data together suggest that anti-AGE1 binds to this amyloid fold, irrespective of the (poly)peptide that bears the cross-β structure fold, which identifies anti-AGE1 as a member of the class of multiligand cross-β structure-binding proteins.

Based on the above-listed results obtained with anti-AGE1, it was tested whether commercially available rabbit anti-human Aβ(1-42) H-43 also displays broad-range specificity towards any (poly)peptide with unrelated amino acid sequence, though with amyloid characteristics. Indeed, with an ELISA, it could be shown that H-43 not only binds to Aβ(1-40), but also to IAPP and albumin-AGE (FIG. 15, Panel H). In addition, binding of H-43 to immobilized IAPP was effectively diminished by tPA (FIG. 15, Panel I). These observations together show that anti-Aβ(1-42) H-43 can bind to other amyloid (poly)peptides in a way similar to multiligand cross-β structure-binding protein tPA.

ELISAs with polyclonal mouse anti-albumin-AGE/Aβ show that the antibody not only binds to these antigens, but that it specifically binds to other amyloid peptides than those used for immunization (FIG. 15, Panels J-L). Similar to the rabbit anti-AGE1 antibody and anti-Aβ(1-42) H-43, anti-albumin-AGE/Aβ displays affinity for the amyloid peptides tested, irrespective of amino acid sequence. This suggests that also mouse anti-albumin-AGE/Aβ is a multiligand amyloid-binding antibody.

Based on the amyloid-binding characteristics of anti-AGE1, anti-Aβ(1-42) H-43 and anti-albumin-AGE/Aβ, the amyloid-binding fraction of anti-AGE2 was purified, which is elicited against albumin-AGE:23, with Aβ fibrils irreversibly coupled to a column. This fraction was used for immunohistochemical analysis of a human brain section that is afflicted with Alzheimer's disease. In FIG. 15, Panel M, it is clearly seen that the antibody binds specifically to the spherical amyloid deposition, indicated by the Congo red fluorescence, shown in FIG. 15, Panel N.

The finding that anti-amyloid and anti-AGE antibodies display affinity for a broad range of sequentially unrelated (poly)peptides, as long as the cross-β structure fold is present, is in agreement with the recently published data by O'Nuallain and Wetzel (O'Nuallain, 2002) and Kayed et al. (Kayed, 2003). From several older reports in literature, it can be distilled that anti-cross-β antibodies can be obtained. For example, cross-reactive antibodies against fibrin and Aβ and against Aβ and hemoglobin are described. It is indicated herein that fibrinogen and hemoglobin-AGE adopt the cross-β structure fold, which suggests that the cross-reactivity observed for anti-Aβ antibodies was in fact binding of anti-cross-β structure antibodies to similar structural epitopes on Aβ, fibrinogen and hemoglobin.

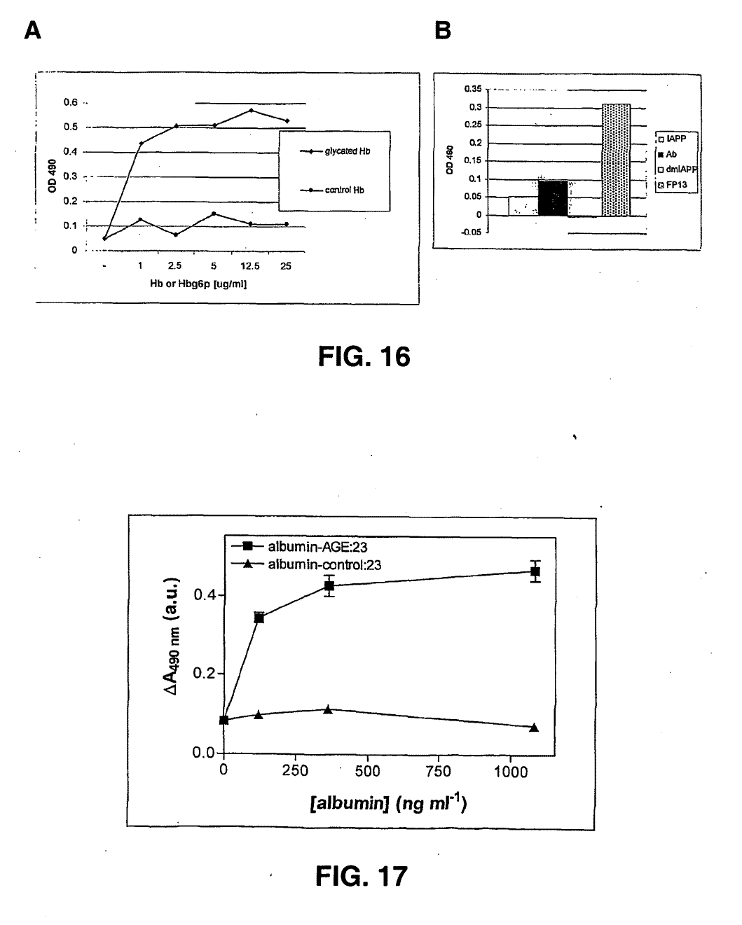

Based on the results with the poly-clonal anti-AGE and amyloid antibodies, it was hypothesized that anti-cross-β structure antibodies could be obtained. Therefore, the spleen of mice immunized with glycated BSA and Aβ with myeloma cells were fused. Potential anti-cross-β structure antibodies were subsequently selected by examining binding of hybridoma produced antibodies to glycated hemoglobin and IAPP. Using this procedure, a monoclonal antibody 3H7 that recognizes glycated hemoglobin, as well as several peptides that contain the cross-β structure (FIG. 16), was isolated. No binding was observed to unglycated hemoglobin or a synthetic peptide that does not form amyloid fibrils (mΔIAPP).

### Example 13

### Sandwich ELISA: Fishing Amyloid Structures from Solution

Using a sandwich ELISA approach with coated tPA that was overlayed with amyloid albumin-AGE:23 in solution, followed by detection with broad-range anti-Aβ(1-42) H-43 (FIG. 17), detection of cross-β structure-containing proteins in solution was successful.

It is herein disclosed that the three-dimensional structures of the tPA finger domain and the fibronectin finger domains 4-5 reveal striking structural homology with respect to local charge-density distribution. Both structures contain a similar solvent-exposed stretch of five amino acid residues with alternating charge; for tPA Arg7, Glu9, Arg23, Glu32, Arg30, and for fibronectin Arg83, Glu85, Lys87, Glu89, Arg90, located at the fifth finger domain, respectively. The charged-residue alignments are located at the same side of the finger module. These alignments may be essential for fibrin binding.

Based on these observations, results and the herein-disclosed similarities, it is shown that the same binding sites for tPA become present in all proteins that bind and activate tPA and that this binding site comprises cross-β structure.

Taken together, the data show that cross-β structure is a physiological relevant quarternary structure element, which appearance is tightly regulated and which occurrence induces a normal physiological response, i.e., the removal of unwanted biomolecules. The existence of a general system, which is termed “cross-β structure pathway,” to remove unwanted biomolecules is disclosed herein for the first time. These results show that this mechanism is fundamental to nature and controls many physiological processes to protect organisms from induced damage or from accumulating useless or denatured biomolecules. If by whatever means deregulated, this system may cause severe problems. On the other hand, if properly used, this system may be applicable for inducing cell death in specific target cells like, for example, tumor cells.

### Example 14

The fibrinolytic cascade and the contact activation cascade of the hemostatic system are triggered by protein aggregates: Tissue-type plasminogen activator and factor XII interact with protein aggregates comprising amyloid-like cross-β structure, via their fibronectin type I domain.

tPA, Factor XII, Fibronectin and the Fibronectin Type I Domains of tPA, Factor XII and Fibronectin Bind to Protein Aggregates With Cross-β Structure Conformation

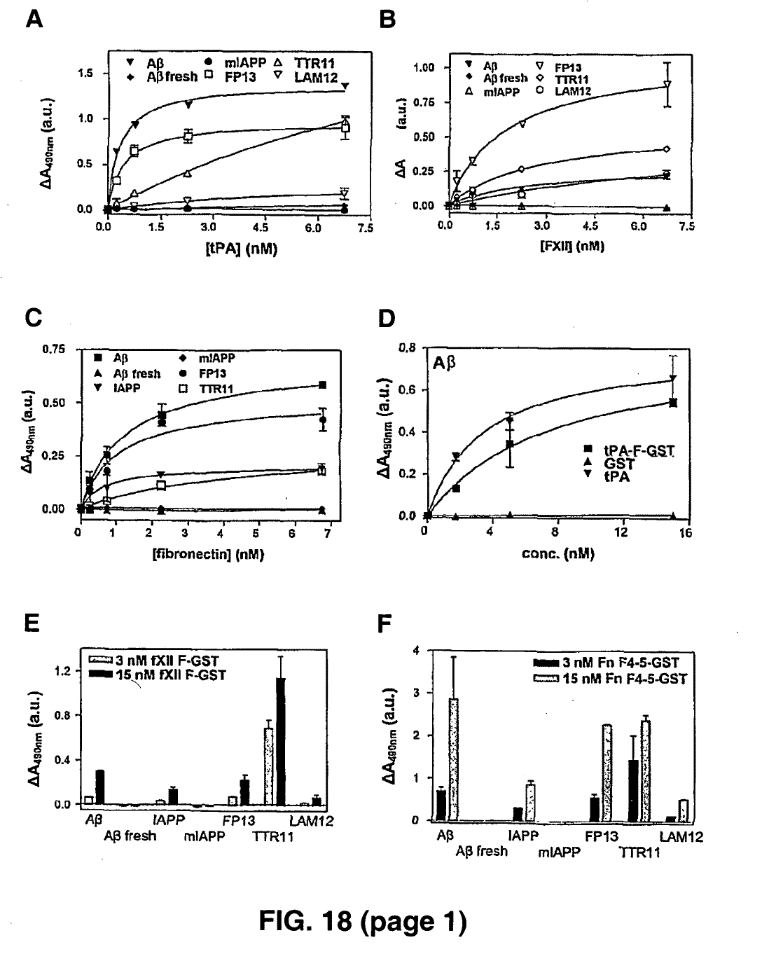

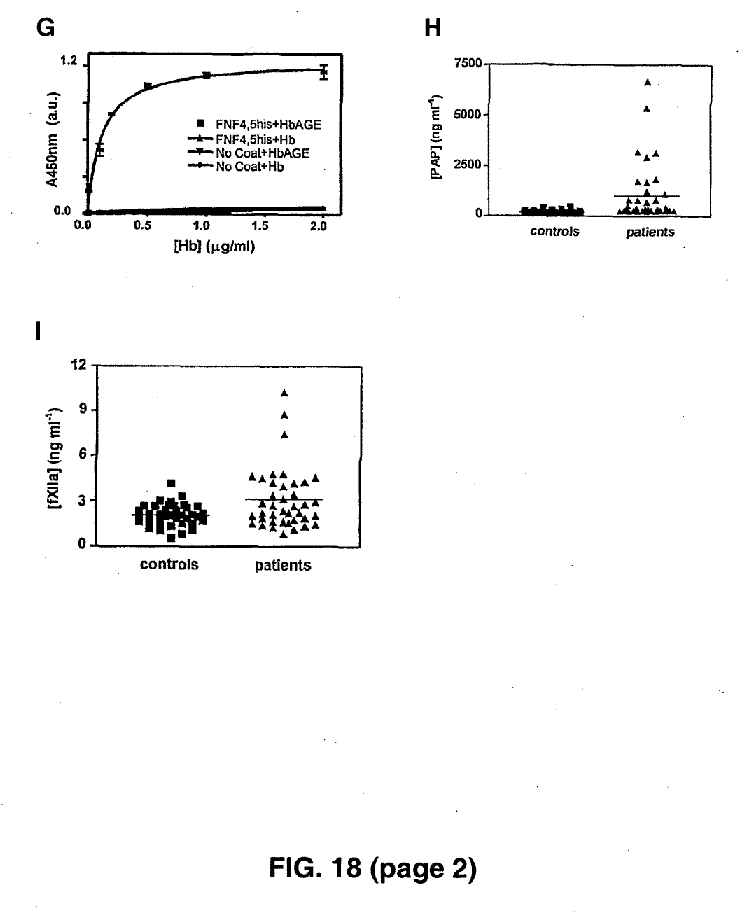

Previously, it was established that tissue-type plasminogen activator interacts with protein and peptide aggregates that comprise the cross-β structure conformation, a structural element found in amyloid-like polypeptide assemblies (Kranenburg, 2002; Bouma, 2003). This analysis has now been expanded to other proteins that resemble tPA domain architecture and to separate domains of tPA. Binding of full-length tPA, factor XII and fibronectin, as well as of fibronectin type I (finger, F) domains of tPA and factor XII and F4-5 of fibronectin, to protein and peptide aggregates with cross-β structure conformation was analyzed in an ELISA. In FIG. 18, it is shown that the full-length proteins, as well as the recombinant F domains, bind specifically to cross-β structure-rich compounds. Binding of tPA and factor XII was established for immobilized amyloid-β(1-40) (Aβ) with amyloid-like properties, fibrin peptide FP13, that encompasses the tPA activating sequence 148KRLEVDIDIKIR160 of the fibrin α-chain, TTR11, which is an 11 amino acid residues peptide from transthyretin that forms cross-β structure, and LAM12, which is a 12 amino acid residues peptide from laminin that forms cross-β structure (FIG. 18, Panels A, B). Negative controls were freshly dissolved, monomerized Aβ and non-amyloid murine islet amyloid polypeptide (mIAPP). For fibronectin, human amyloid IAPP is depicted instead of LAM12 (FIG. 18, Panel C). The separate F domains also bind to aggregates with cross-β structure, as depicted for Aβ and tPA-F in FIG. 18, Panel D, and for all aggregates and factor XII F and fibronectin F4-5 in FIG. 18, Panels E and F. In addition, immobilized fibronectin F4-5 with a His-tag specifically captures glycated hemoglobin with amyloid-like properties in solution (FIG. 18, Panel G).

Activation of Factor XII and tPA by Protein Aggregates with Amyloid-Like Cross-β Structure

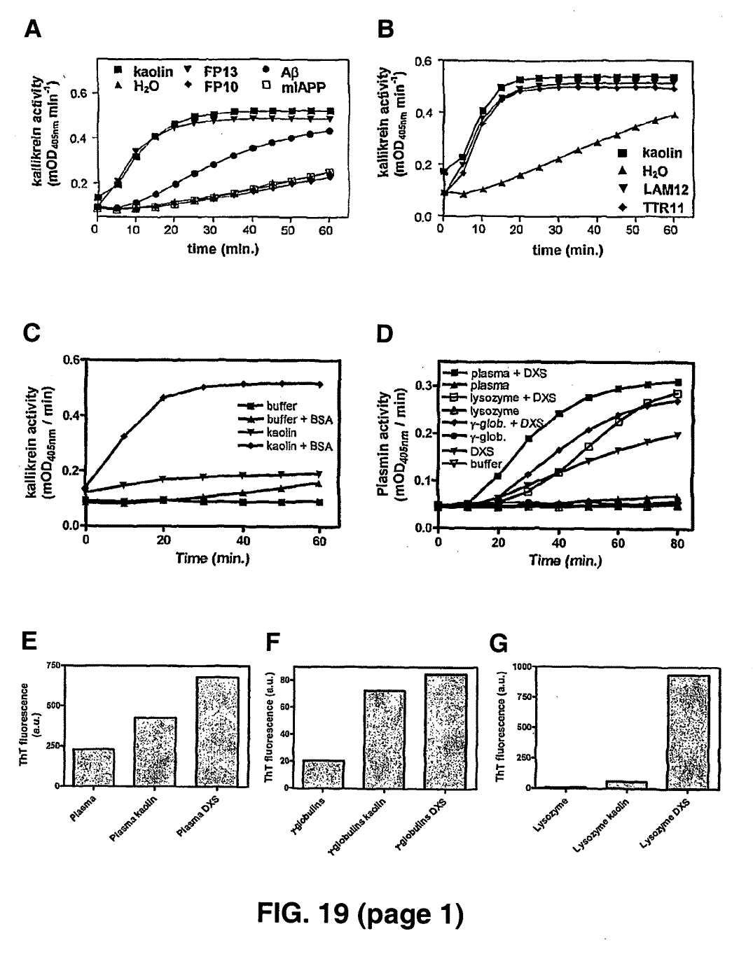

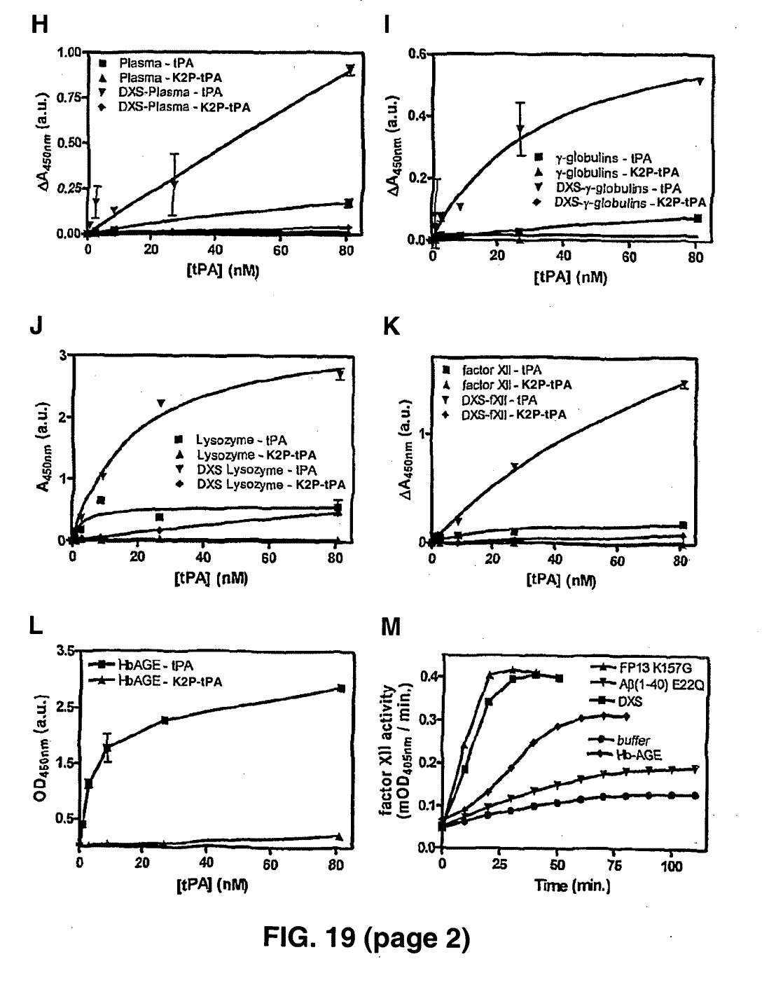

Contacting factor. XII to artificial negatively charged surfaces results in its activation, as measured by the conversion of prekallikrein to kallikrein, which can convert chromogenic substrate Chromozym PK (FIG. 19). Now, it is demonstrated that peptide aggregates with cross-β structure conformation, the protein conformation found in amyloid, also stimulate factor XII activation (FIG. 19). Moreover, it is demonstrated that kaolin is able to stimulate factor XII activation only when a protein cofactor, e.g., albumin, is present in the assay buffer (FIG. 19, Panel C). Contacting another established factor XII activating surface, i.e., dextran sulphate 500,000 Da (DXS500k), with various proteins, including lysozyme, γ-globulins, whole plasma and factor XII itself, results in the introduction of amyloid-like properties in the proteins, e.g., activation of tPA (FIG. 19, Panel D), binding of Thioflavin T (FIG. 19, Panels E-G) and binding of tPA (FIG. 19, Panels H-K), indicative for the formation of cross-β structure in the protein aggregates after exposure to the negatively charged surface. The ability of protein aggregates with cross-β structure conformation to induce auto-activation of factor XII was also tested. For this purpose, purified factor XII was incubated with substrate S-2222 and either buffer, or 1 μg/ml DXS500k, 100 μg/ml FP13 K157G, 10 μg/ml Aβ(1-40) E22Q and 10 μg/ml Hb-AGE. All three amyloid-like aggregates are able to induce factor XII auto-activation (FIG. 19, Panel M). FP13 K157G and Hb-AGE have a potency to induce auto-activation that is similar to the established surface activator DXS500k, whereas the potency of the Aβ(1-40) E22Q is somewhat lower.

The aforementioned observations show that both the contact system and the fibrinolytic system are activated in conformational diseases, or amyloidoses. Activation of these systems in patients with systemic amyloidoses was measured. Increased activation of the fibrinolytic system is detected by measuring the levels of plasmin in complex with its circulating inhibitor α2-anti-plasmin (PAP). Increased activity of the contact system is detected by measuring the levels of activated factor XII. Both PAP levels (3192 ng ml⁻¹ vs 217 ng ml⁻¹) and factor XIIa levels (3.1 ng ml⁻¹ vs 2.1 ng ml⁻¹) were elevated in patient plasma (FIG. 18, Panels H, I). Twelve out of 16 patients with elevated factor XIIa levels also had elevated PAP levels. These measurements show that systemic amyloidosis is accompanied by increased levels of activated serine proteases, and disclose a role for amyloid in activation of tPA and factor XII in vivo.

Factor XII, tPA, Fibronectin and their Recombinant Fibronectin Type I Domains Interact with Aggregates Comprising Cross-β Structure

Like tPA, factor XII, fibronectin, tPA-F domain, factor XII F domain and fibronectin F4-5 domains bind to peptide aggregates with cross-β structure conformation. In addition, a chemically synthesized F domain of tPA (T. Hackeng, Academic hospital Maastricht, The Netherlands), the fibronectin F10-12 domains and the hepatocyte growth factor activator F domain bind to amyloid-like cross-β structure-rich aggregates (B. Bouma, data not shown). Moreover, like tPA, factor XII becomes activated by amyloid-like aggregates. This has not only been established in an indirect way by measuring activated kallikrein from prekallikrein upon activation of factor XII, but also in a direct way by measuring auto-activation of factor XII upon exposure to amyloid-like protein aggregates (see FIG. 19). The data also indicate that several negatively charged surfaces that are well known for their ability to activate factor XII, i.e., kaolin and DXS500k, need a protein cofactor to gain stimulatory capacities. Binding of Thioflavin T and tPA after exposure of proteins to DXS500k show that the protein aggregate cofactors adopt the cross-β structure conformation, that is essential for both the factor XII activation and the tPA activation.

The data show that both the fibrinolytic cascade and the contact system of blood coagulation become activated by activation of tPA and factor XII via protein aggregates with amyloid-like cross-β structure conformation, respectively. Moreover, the presence of amyloid-like protein conformation in the circulation or elsewhere in the body is a risk factor for inducing pathological activation of the fibrinolytic cascade and/or the contact activation system.

The data on factor XII activation allow for a further analysis of the role of cross-β structure in factor XII activation. For example, factor XII auto-activation by cross-β structure is analyzed by contacting purified factor XII to cross-β structure in the presence of a chromogenic substrate that is converted when factor XII is activated. In addition, the influence of cross-β structure-binding proteins and compounds on the activation of factor XII in the presence of cross-β structure is studied. The observation that both tPA and factor. XII become activated by proteins that are contacted to DXS500k further show that the fibrinolytic cascade and the contact activation cascade of the hemostatic system is activated by a common mechanism, in which protein aggregates comprising amyloid-like cross-β structure play an initiating role.

The data show increased levels of PAP and fXIIa in amyloidosis patients. These results show a role for amyloid-like protein aggregates in the activation of tPA and factor XII in the amyloidosis patients. These results also provide a molecular explanation for bleeding diathesis and coagulation abnormalities that are recognized complications in amyloidosis patients. Dysregulation of the fibrinolytic system and the contact system by amyloid underlies this pathology directly by amyloid-mediated activation of tPA and indirectly by factor XII-mediated formation of bradykinin, which releases tPA. In addition to a role for fibronectin type I domain-comprising tPA and factor XII in amyloidosis, a role for HGFA and fibronectin in conformational diseases is clear. Incorporation of fibronectin in amyloid deposits serves a function in neurite outgrowth in Alzheimer's disease, and represents a defense mechanism against amyloid deposition by shielding of toxic structures and by inhibiting fibril extension. Hepatocyte growth factor (HGF, scatter factor), a physiological substrate of HGFA, is increased in the brain of AD patients and HGF levels in plasma are elevated in amyloid A amyloidosis and light-chain amyloidosis. The data now show that fibronectin type I-amyloid interactions are important for these activities.

### Example 15

Isolated blood platelets become activated via their p38MAPK pathway and aggregate upon exposure to polypeptides with cross-β structure conformation.

Blood Platelets Become Activated and Aggregate Upon Exposure to Proteins with Cross-β Structure Conformation, Express Amyloid-Like Structures and Show Increased Binding of Amyloid Dye Thioflavin T Upon Aging

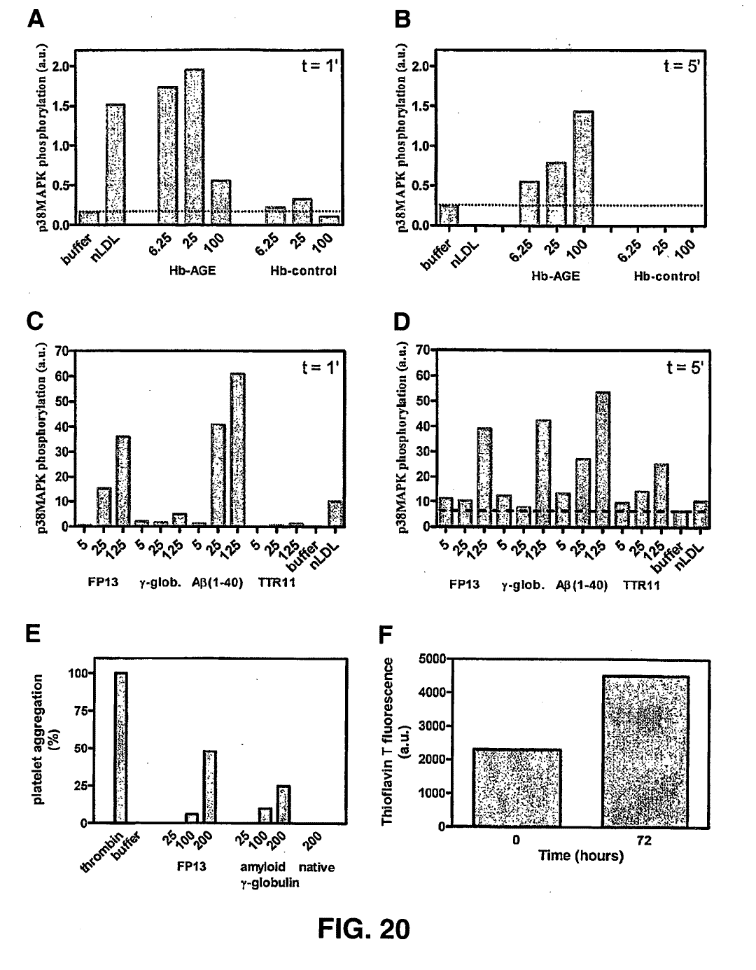

Incubation of freshly isolated platelets with various compounds that contain cross-β structure conformation results in activation of the p38MAPK pathway, as determined by analysis of p38MAPK phosphorylation. Incubation of platelets with amyloid hemoglobin-AGE results in platelet activation similar to the positive control native low-density lipoprotein after one minute (FIG. 20, Panel A). After five minutes, Hb-AGE shows a prolonged activation, whereas p38MAPK is not phosphorylated by nLDL stimulation anymore (FIG. 20, Panel B). Incubation with control hemoglobin results in background levels of p38MAPK phosphorylation, similar to buffer. Amyloid peptides FP13 and Aβ already potently induce p38MAPK phosphorylation after one minute incubation (FIG. 20, Panel C), whereas amyloid denatured γ-globulins and transthyretin amyloid fragment TTR11 induce p38MAPK phosphorylation only after five minutes stimulation (FIG. 20, Panel D). In a blood platelet aggregometer, the influence of amyloid FP13 and denatured γ-globulins was tested and compared to the effect of thrombin on aggregation. Negative controls were HEPES-Tyrode buffer and 200 μg/ml native γ-globulins. Both FP13 and denatured γ-globulins with amyloid-like conformation induce platelet aggregation in a dose-dependent manner (FIG. 20, Panel E). In a separate experiment, the influence of platelet aging on Thioflavin T binding upon storage at room temperature was assayed. After 72 hours, Thioflavin T fluorescence was approximately doubled, showing an increase in the amount of cross-β structure (FIG. 20, Panel F).

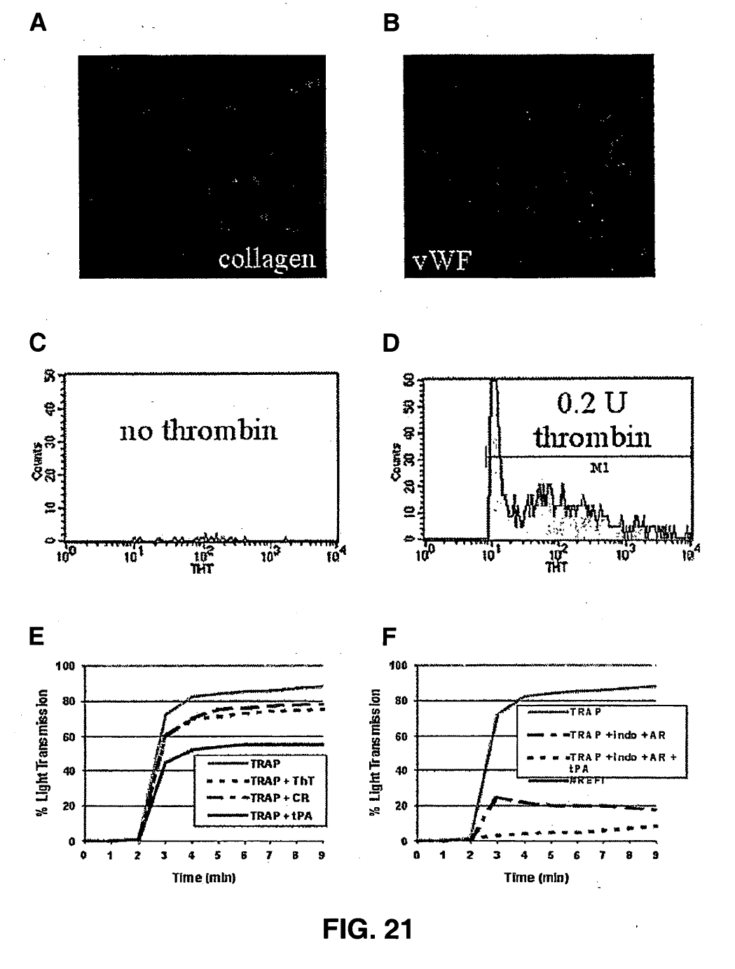

Recent insights have indicated that the formation of amyloid is not necessarily the result of a defect in the normal folding or clearance pathway, but that amyloid is also formed through normal biological proteolytic processing. It was found that (i) activation of platelets induces amyloid at their cell surface and (ii) that platelets adhered to von Willebrand Factor (vWF) or collagen surface under flow express amyloid domains (FIG. 21, Panels A, B). Expression is at the cell body and areas of spreading are negative (vWF surface) and at tips of aggregates (collagen), suggesting that adhered and aggregated platelets express areas of rich and poor in amyloid. Platelets stimulated with TRAP (an activator of the PAR-1 receptor without proteolytic properties and incapable of converting released fibrinogen into fibrin) and thrombin (an activator of PAR-1 and PAR-4 through proteolysis and an activator of fibrin formation) express amyloid as visualized using the fluorescent amyloid dyes Congo Red and ThT in a FACS analysis (FIG. 21, Panels C, D). Resting platelets do not express amyloid at the cell surface (FIG. 21, Panel C).

To test whether amyloid may influence platelet aggregation by classical stimuli, it was tested whether amyloid-specific dyes and the amyloid-binding protein tPA affect platelet aggregation. Indeed, using optical aggregometry, it was observed that Congo Red, ThT, as well as tPA, inhibited platelet aggregation. Dose response studies show up to 30% inhibition by 200 μM Congo red and up to 45% inhibition by 200 μM ThT (FIG. 4, Panel-E). tPA (1 μM) even induced 55% inhibition of thrombin-induced aggregation. The inhibition persisted in platelets treated with indomethacin (aspirin-like) and AR-C6993MX (clopidogrel-like), indicating that amyloid contributed to platelet aggregation via mechanisms independent of thromboxane A₂ formation or P2Y12 stimulation through released ADP (FIG. 21, Panel F).

### Cross-β Structure is Thrombogenic

Based on the observations that compounds that comprise cross-β structure induces activation in platelets of the p38MAPK pathway and induces platelet aggregation, it was concluded that the exposure of platelets to proteins with cross-β structure in the circulation has similar effects. Platelets stored under conditions recommended by the blood bank show increased binding of an amyloid dye. Therefore, aging platelets themselves may contain proteins that have adopted the activating cross-β structure conformation. Isolating only those cells in donor blood that display relatively minimal ThT binding is beneficial for the acceptor of the platelets.

The pilot studies have demonstrated the presence of amyloid on activated and adhered platelets. For the development of effective anti-thrombogenic agents, knowledge on which protein or proteins are forming amyloid-like structures on platelets is required. In addition, knowledge on which pathways are involved in extracellular exposure upon platelet activation of putatively intracellularly stored amyloid-like conformation-rich proteins, is beneficial for the development of treatment strategies that prevent amyloid exposure on platelets. With this knowledge, pathway inhibitors are developed solely or in addition to blockers/scavengers of amyloid-like aggregates.

### Example 16

Relationship between the structure of β2-glycoprotein I, the key antigen in patients with Antiphospholipid syndrome, and antigenicity

### The Anti-Phospholipid Syndrome and Conformationally Altered β2-Glycoprotein I

The anti-phospholipid syndrome (APS) is an auto-immune disease characterized by the presence of anti-β2-glycoprotein I auto-antibodies. Two of the major clinical concerns of the APS are the propensity of auto-antibodies to induce thrombosis and the risk for fetal resorption. Little is known about the onset of the auto-immune disease. Recent work has demonstrated the need for conformational alterations in the main antigen in APS, β2-glycoprotein I (β2gpi), before the initially hidden epitope for auto-antibodies is exposed (de Laat, 2004; de Laat, 2005; Matsuura, 1994). Binding of native β2gpi to certain types of ELISA plates mimics the exposure of the cryptic epitopes that are apparently present in APS patients. It has been demonstrated that anti-β2gpi auto-antibodies do not bind to globular β2gpi in solution, but only then when β2gpi has been immobilized to certain types of ELISA plates (de Laat, 2004; de Laat, 2005; Matsuura, 1994). Thus, the globular and native form of the protein is not the primary antigen in the autoimmune disease. Auto-antibodies seem to be elicited against a conformationally altered form of autologous β2gpi, which would fit in the “danger” model of immunology (Matzinger, 2002a; Matzinger 2002b). This proposed mechanism could explain the observed risk for thrombosis in APS patients. Auto-antibodies prevent clearance of conformationally altered β2gpi with exposed amyloid cross-β structure epitopes. This induces activation of the contact system, platelet activation and tissue factor expression on endothelial cells.

Factor XII and tPA Bind to Recombinant β2gpi and to β2gpi Purified from Frozen-Thawed Plasma, and not to β2gpi Purified from Fresh Plasma

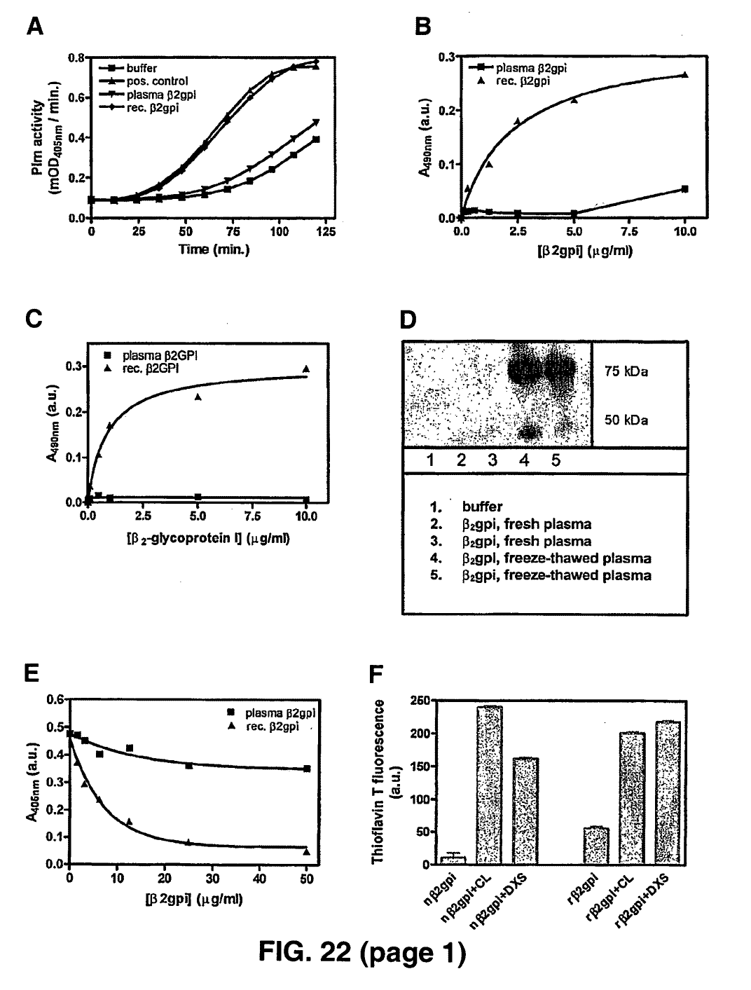

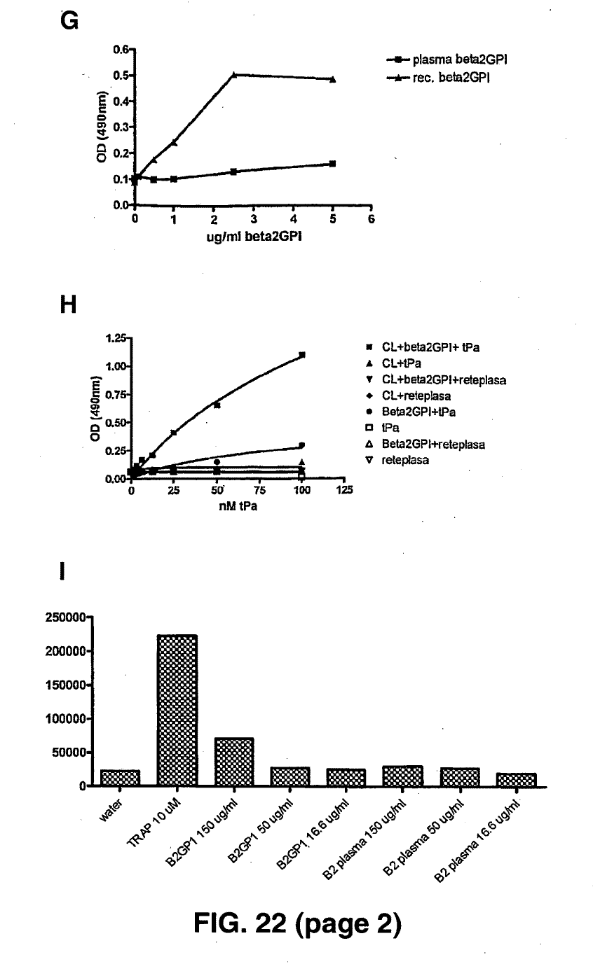

Recombinant β2gpi and not β2gpi purified from fresh plasma, stimulates effectively the tPA-mediated conversion of plasminogen to plasmin, as measured as the conversion of the plasmin-specific chromogenic substrate S-2251 (FIG. 22, Panel A). Factor XII and tPA do not bind to β2gpi purified from fresh human plasma (FIG. 22, Panels B, C). Recombinant β2gpi, however, binds to factor XII with a k_{D} of 20 nM and to tPA with a k_{D} of 51 nM (FIG. 22). In addition, when β2gpi is purified from plasma that was frozen at −20° C. and subsequently thawed, factor XII co-elutes from the anti-β2gpi antibody affinity column, as shown on Western blot after incubation of the blot with anti-factor. XII antibody (FIG. 22, Panel D). In FIG. 22, Panel E, the inhibitory effect of recombinant β2gpi on binding of anti-β2gpi auto-antibodies isolated from patients with anti-phospholipid syndrome to immobilized β2gpi is shown. It is shown that plasma-derived β2gpi in solution has no effect on the antibody binding to immobilized β2gpi. FIG. 22, Panel F shows that exposure of β2gpi to cardiolipin or dextransulphate 500,000. Da introduces an increased ThT fluorescence signal, indicative for a conformational change in β2gpi accompanied with the formation of cross-β structure. Again, recombinant β02gpi initially gave a higher Thioflavin T fluorescence signal than native β2gpi purified from plasma. In addition, tPA binds with higher affinity and to a higher extent to β2gpi bound to immobilized cardiolipin, than to β2gpi that is directly immobilized on wells of an ELISA plate (B. de Laat, data not shown). These observations also show that cardiolipin has a denaturing effect, thereby inducing amyloid-like conformation in β2gpi, necessary for tPA binding.

Binding of recombinant β2gpi and β2gpi purified from plasma to tPA has also been assessed in an alternative set-up. TPA was immobilized onto the wells of an ELISA plate and subsequently overlayed first with concentration series of recombinant β2gpi or of plasma β2gpi, and an anti-β2gpi antibody (FIG. 5, Panel-G). In FIG. 5, Panel H, it is shown that exposure of β2gpi to cardiolipin, immobilized on the wells of an ELISA plate, renders β2gpi with tPA-binding capacity. Binding of β2gpi directly to the ELISA plate results in less tPA binding. These observations, together with the observation that exposure of β2gpi to cardiolipin vesicle-induced ThT-binding capacity (FIG. 22, Panel F), show that introducing β2gpi to a denaturing surface induces formation of amyloid-like cross-β structure conformation.

In FIG. 20, it is shown that blood platelets are activated and aggregate upon exposure to protein aggregates with cross-β structure. Therefore, it was tested whether recombinant β2gpi, that shows properties reminiscent to an aggregate with cross-β structure conformation, and β2gpi purified from human plasma, are able to induce platelet activation (FIG. 22, Panel I). Exposure of platelets to recombinant β2gpi results in somewhat higher phosphorylation of p38MAPK. From these results, it is concluded that β2gpi exposing the amyloid-like cross-β structure conformation may serve as an activator of platelets, resulting in platelet aggregation. In this way, β2gpi is turned into a thrombogenic protein, giving a rationale to the observed thrombogenic activity seen in patients with the APS.

### Epitopes for Auto-Antibodies are Specifically Exposed on Non-Native Conformations of β2gpi Comprising Cross-β Structure

FIG. 22 shows that preparations of β2gpi react with amyloid cross-β structure markers. In addition, exposure of β2gpi to cardiolipin introduces tPA binding capacity (data not shown). The β2gpi preparations with cross-β structure conformation express epitopes that are recognized by anti-β2gpi auto-antibodies isolated from APS patient plasma. Furthermore, exposure of β2gpi to cardiolipin or dextran sulphate 500,000 Da induces an increased fluorescence when ThT is added, indicative for the formation of cross-β structure when β2gpi contacts a negatively charged surface. Interestingly, it has previously been observed that exposure of β2gpi to cardiolipin is a prerequisite for the detection of anti-β2gpi antibodies in sera of immunized mice (Subang, 2000). These combined observations point to a role for conformational changes in native β2gpi, necessary to expose new immunogenic sites. The cross-β structure element is part of this initially absent epitope. To further establish this view, immunization studies with native β2gpi and conformationally altered β2gpi, with or without cross-β structure, can be performed. Sources of conformationally altered β2gpi are recombinant β2gpi, or β2gpi exposed to any denaturing surface, e.g., cardiolipin and DXS500k. In vitro cellular assays and in vivo mouse models help to gain insight into the putative role of the cross-β structure in auto-immunity. Cross-reactivity of antibodies directed against conformationally altered β2gpi with certain pathogens adds to the combined ideas that early episodes of pathogen infection induce anti-β2gpi auto-antibodies and to the newly disclosed hypothesis that anti-β2gpi auto-antibodies are raised against a form of β2gpi with cross-β structure. Antibodies against the pathogens are putatively directed to amyloid-like proteins, present at the surface (Gebbink, 2005), which would then explain cross-reactivity with host proteins comprising cross-β structure, including β2gpi.

With the observations that support the idea that cross-β structure in part build up an epitope recognized by autoimmune antibodies, the studies are expanded to other diseases and complications in which auto-antibodies play a role. For example, hemophilia patients with anti-factor VIII auto-antibodies are screened for the presence of antibodies in their plasma that recognize the cross-β structure conformation. A more detailed analysis reveals whether putative cross-β structure-binding antibodies specifically bind in part to cross-β structure in the antigen, or whether the antibodies bind to cross-β structure present in any unrelated protein.

### Example 17

### The Artherogenic Form of Low-Density Lipoproteins, Oxidized LDL, Exhibits Amyloid-Like Structural Properties

### Oxidation of Low-Density Lipoprotein Particles Contributes to the Pathogenesis of Artherogenesis

Misfolding and aggregation of the apoB protein fraction of LDL upon oxidation, its resistance to proteolysis and its cytotoxicity are motifs commonly seen for amyloid-like protein aggregates. Moreover, multiligand receptors with affinity for amyloid-like structures, i.e., CD36, RAGE, scavenger receptor A and scavenger receptor B-I, are also receptors for oxidized adducts of lipoproteins. Therefore, it has been suggested that the role of amyloid-like apoB in oxidized LDL particles during atherosclerosis is similar to the pathogenic role of amyloid-like aggregates in protein misfolding diseases.

### Oxidized LDL Displays Amyloid-Like Features Pointing to the Presence of Cross-β Structure Conformation

Freshly isolated low-density lipoprotein (LDL) was oxidized upon incubation with 25 μM CuSO₄, for various incubation times. In time, the degree of oxidation was determined by reading the absorbance of diene structures at 234 nm, as well as the fluorescence upon incubation of oxidized LDL (oxLDL) with Congo red or Thioflavin T (FIG. 23. Panels A, B). With a 24% oxidized oxLDL preparation, the ability to activate tPA in the chromogenic plasmin activation assay was determined and compared to native LDL. It was clearly seen that upon oxidation, LDL gains tPA activating properties (FIG. 23, Panel C). In an additional experiment, the ability of oxidized LDL to activate factor XII in plasma was determined in a chromogenic assay using substrate S-2222. Like amyloid fibrin-derived peptide FP13 K157G, oxLDL stimulates the conversion of S-2222, indicative for the ability of oxLDL to induce factor XII activation (FIG. 23, Panel D).

The Pathological Role of oxLDL During Atherosclerosis is Related to the Presence of Amyloid-Like Cross-β Structure Conformation in the apoB Fraction

Previous data show that oxLDL plays an important role in the pathological events seen during atherosclerosis. The role of the interaction between oxLDL particles and multiligand receptor CD36 on the surface of blood platelets has been demonstrated. CD36 is one of the known cellular receptors with broad range ligand specificity, including specificity for amyloid-like structures. The data now add to the idea that the cross-β structure conformation in oxLDL, as well as in amyloid-β and in glycated proteins, is the true ligand binding site that is important in mediating signals outside/in the platelets. Activation of platelets by oxLDL results in platelet aggregation that contributes to the thrombogenic conditions seen during atherosclerosis. The observations that amyloid-like protein aggregates with cross-β structure conformation are also able to activate platelets and to induce platelet aggregation fit in the idea that protein aggregates comprising amyloid-like structures, including oxLDL, play a pivotal role in the pathological conditions that come with thrombogenesis. Moreover, the data show that oxLDL contributes to pathological conditions via two additional ways. First, the fibrinolytic cascade is activated by inducing tPA activation. Second, oxLDL activates the contact system of blood coagulation by inducing factor XII activation. The ability of oxLDL to both activate tPA and factor XII is similar to what is observed with many of the amyloid-like peptides and proteins with cross-β structure. Therefore, this ability of oxLDL points to the presence of cross-β structure in the apoB protein part of the oxidized LDL particles.

### Example 18

### A Fibrin Clot Comprises Amyloid-Like Cross-β Structure Conformation

A Fibrin Clot Binds Amyloid-Specific Dyes Congo red, Thioflavin T and Thioflavin S

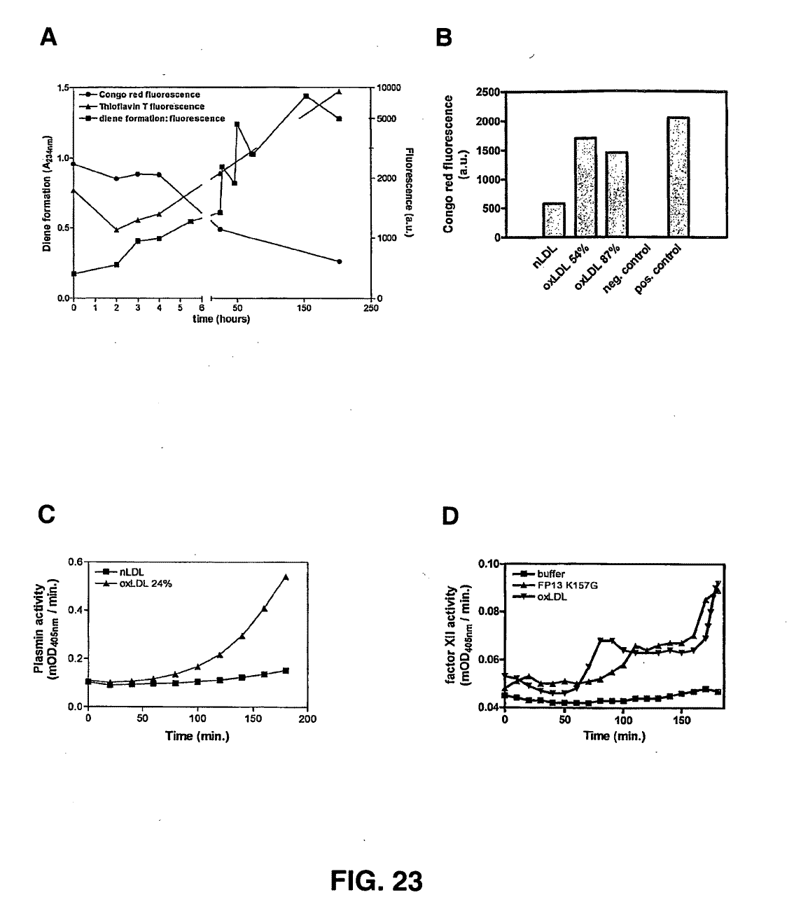

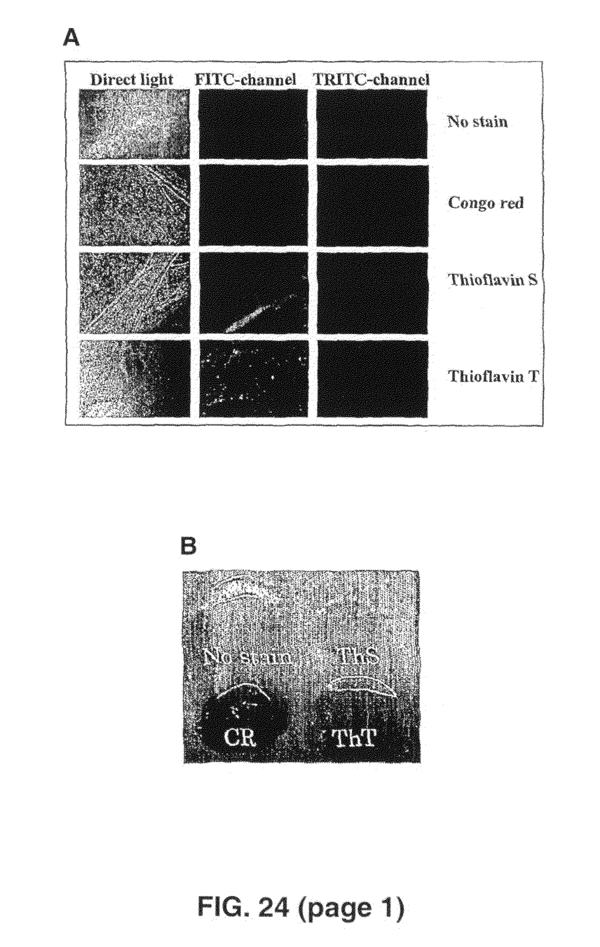

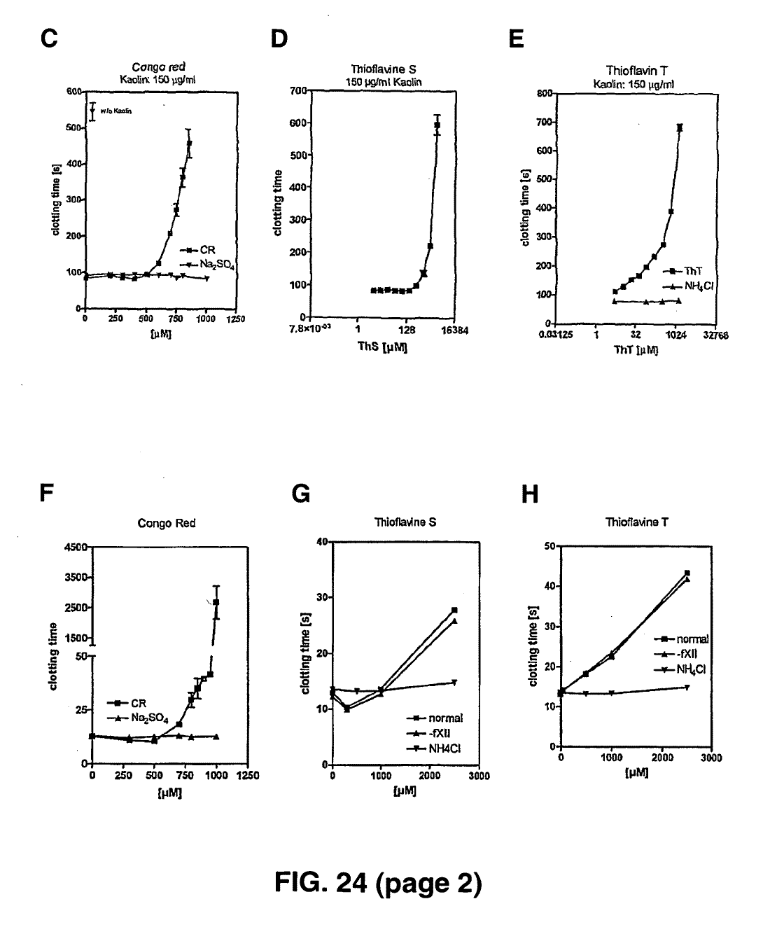

Incubation of a preformed fibrin clot with the amyloid-specific dyes Congo red (CR), Thioflavin S (ThS) or Thioflavin T (ThT) results in specific binding of the dyes, as assayed with direct-light microscopy and fluorescence microscopy (FIG. 24, Panels A, B). In addition, formation of a fibrin clot is delayed in the presence of the amyloid-specific dyes, as established both in aPTTs (FIG. 24, Panels C-E) and in PTs (FIG. 24, Panels F-H). These data indicate that a fibrin clot is composed of aggregates comprising the amyloid-specific cross-β structure conformation. Moreover, the data show that formation of a three-dimensional fibrin polymer network is dependent on cross-β structure formation, as introduction of amyloid-binding dyes inhibit fibrin assembly into clots. The previously disclosed observations (see above) showed that platelet activation and aggregation is induced by cross-β structure-rich activators (FIG. 20). Moreover, it is shown that platelet activation is accompanied by the surface exposure of amyloid-like aggregates of proteins with cross-β structure conformation (FIG. 21). Combined with the observation that a fibrin clot comprises cross-β structure conformation, it is now obvious that polymerized fibrin in a blood clot serves as the cross-β structure-rich source for (further) platelet activation.

### Examples 19-30

### Introduction

### Role of Crossbeta Structure at the Surface of Pathogens in Triggering the Hemostatic System and the Fibrinolytic System

During infection with, for example, pathogens, such as bacteria, fungi, parasites and viruses, components of the hemostatic system and the fibrinolytic pathway are activated. Proteins with a crossbeta structure conformation at the surface of various pathogens mediate infection. Examples 19-30 provide a number of experiments, such as cell-based bioassays, blood enzyme activation tests and coagulation tests with a series of pathogens to provide evidence that compounds capable of specifically interacting with crossbeta structures are suitable to, at least in part, inhibit and/or prevent and/or counteract and/or abolish and/or reverse and/or diminish and/or interfere with activating properties of pathogens towards the hemostatic and fibrinolytic systems. The experiments provide leads for therapeutics for treatment of an undesired blood coagulant state, based on crossbeta structures and/or crossbeta structure-binding compounds.

### Pathogens

Amyloid core proteins have been identified as being part of the core of several classes of pathogens. Those pathogens with cross-beta structure at their surface provide suitable models to analyze the role of cross-beta structure-comprising proteins in hemostasis.

### Determination of the Presence of Crossbeta Structure Protein Conformation on Pathogens

With pathogens, a series of analyses is performed that provides insight in the presence of crossbeta structure protein conformation. Standard Congo red and Thioflavin T-binding assays are conducted. Interaction with crossbeta structure-binding proteins, tissue-type plasminogen activator, and factor. XII is assessed using chromogenic assays. For this purpose, concentration series of the pathogens are mixed with 100-1000 pM tPA, 5-200 μg/ml plasminogen and 0.1-1 mM chromogenic plasmin substrate S2251 (Chromogenix), and conversion of plasminogen to plasmin upon tPA activation by crossbeta structure is followed in time during 37° C.-incubation. For factor XII activity measurement, concentration series of pathogens are mixed with 0.1-50 μg/ml factor XII, 0-5 μg/ml prekallikrein, 0-5 μg/ml high molecular weight kininogen, and either chromogenic factor XII substrate S2222 (Chromogenix) for direct measurement of factor XII activity, or chromogenic kallikrein substrate Chromozym-PK (Boehringer-Mannheim) for indirect factor XII activity, and substrate conversion is followed in time spectrophotometrically during 37° C. incubation. All of the above-listed analyses are preferably performed with solutions before and after centrifugation for one hour at 100,000*g, or preferably before and after filtration using a 0.2 μm filter. Positive controls that are preferably included in the assays are glycated hemoglobin, amyloid-β and amyloid γ-globulins, prepared by incubation of γ-globulins in H₂O at 37° C., after dissolving lypohilized γ-globulins in 1,1,1,3,3,3-hexafluoro-2-propanol and trifluoro acetic acid, followed by air-drying.

### Crossbeta Structure-Binding Compounds Used as Potential Inhibitors of Crossbeta Structure-Mediated Induction of a Pro-Coagulant State

To be able to analyze the role of crossbeta structure-comprising proteins on pathogens in hemostasis, a series of crossbeta structure-binding compounds is included in the assays as potential inhibitors of crossbeta structure-mediated effects on hemostasis. Crossbeta structure-binding compounds are included in the assays at concentrations of 1-5000 μg/ml, or 1 nM-1 mM. Examples of crossbeta structure-binding compounds that are used are Congo red, Thioflavin T, Thioflavin S, tPA, factor XII, fibronectin, finger domains derived from tPA, factor XII, fibronectin or HGFA, sRAGE, sLRP, LRP cluster 2, LRP cluster 4, (hybridoma) antibodies, IgIV (a fraction that is either not enriched or is enriched by applying a crossbeta structure affinity column), soluble extracellular fragment of LOX-1, or molecular chaperones like, for example, clusterin, haptoglobin, BiP/grp78, HSP60, HSP70, HSP90, gp96 (see Tables 4-5 for more examples of crossbeta structure-binding compounds).

### ABBREVIATIONS

aPTT, activated partial thromboplastin time; BCA, Bicinchoninic Acid; BiP/grp78, Immunoglobulin heavy: chain-binding protein/Endoplasmic reticulum lumenal Ca²⁺-binding protein; cbs, crossbeta structure; CD, Cluster of Differentiation; CFA, colonization stimulating factor; DC, dendritic cell; DMEM, Dulbecco's Modified Eagle Medium; EC, endothelial cell; E. coli, Escherichia coli; ELISA, enzyme-linked immunosorbent assay; F, finger domain/fibronectin type I domain; FCS, fetal calf serum; Fn, fibronectin; HBS, HEPES-buffered saline; HEPES, {2-(4-(2-Hydroxyethyl)-1-piperazinyl)ethanesulfonic Acid}; HGFA, hepatocyte growth factor activator; HMWK, high molecular weight kininogen; HSP, heat-shock protein; HLA-DR, D-related human leukocyte antigen; HUVEC, human umbilical vein endothelial cell; IgIV, immunoglobulins intravenous; IMDM, Iscove's Modified Dulbecco's Medium; IL, interleukin; IvIG, intravenous immunoglobulins; IFN, interferon; LB, luria broth; LRP (═CD91) low-density lipoprotein receptor-related protein; LOX, lecton-like receptor for oxidized low-density lipoprotein; MHC, human leukocyte antigen; MTT, mitochondrial metabolic activity; MFI, mean fluorescent intensity; NO, nitric oxide; PBMC, peripheral blood mononuclear cells; PBS, phosphate-buffered saline; PE, phycoerythrin; PRP, platelet-rich plasma; PT, prothrombin time; PMA, phorbol 12-myristate 13-acetate; RPMI, Roswell Park Memorial Institute; ROS, reactive oxygen species; S. aureus, Staphylococcus aureus; S. pyogenes, Streptococcus pyogenes; sRAGE, soluble fragment of receptor for advanced glycation endproducts; TBS, Tris(hydroxymethyl)aminomethane Hydrochloride-buffered saline; ThT, thioflavin T; TLR, Toll-like receptor; TNF-α, tumor necrosis factor-α; TRAP, synthetic thrombin receptor activating peptide; TPA, Tetra-Phorbol-Acetate; tPA, tissue-type plasminogen activator; ULS, universal linkage system.

### Materials and Methods

### Cloning, Expression and Purification of the Soluble Extracellular Domains of Receptor for Advanced Glycation Endproducts

The soluble extracellular part of the receptor for AGE (sRAGE) was cloned, expressed and purified as, follows (Q.-H. Zeng, Prof. P. Gros, Dept. of Crystal and Structural Chemistry, Bijvoet. Center for Biomolecular Research, Utrecht University, Utrecht, The Netherlands). Human cDNA of RAGE was purchased from RZPD (clone IRALp962E1737Q2, RZPD, Berlin, Germany). For PCRs, the gagatctGCTCAAAACATCACAGCCCGG (SEQ ID NO:34) forward primer was used comprising a BglII site, and the gcggccgcCTCGCCTGGTTCGATGATGC (SEQ ID NO:35) reverse primer with a NotI site. The soluble extracellular part of RAGE comprises three domains spanning amino acid residues 23-325. The PCR product was cloned into a pTT3 vector containing an amino-terminal His-tag and a thrombin cleavage site. The sRAGE was expressed in 293E hamster embryonic kidney cells at the ABC-protein expression facility (Utrecht University, Utrecht, The Netherlands). Concentrated cell culture medium was applied to a Hi-trap Chelating HP Ni²⁺-NTA column (Amersham Biosciences Europe, Roosendaal, The Netherlands). The running buffer was 25 mM Tris-HCl, 500 mM NaCl, pH 8.0. The protein was eluted by using a step gradient of 0 to 500 mM imidazole. Purity of the His-sRAGE was depicted from Coomassie stained SDS-PAGE gels. After concentration, the buffer was exchanged to 20 mM Tris-HCl, 200 mM NaCl, 100 μM phenylmethylsulfonyl fluoride (PMSF), pH 8.0. Various stocks at 1, 5 and 20 mg ml⁻¹ were first kept at 4° C. for several weeks and then stored at −20° C. In this way, the PMSF will be sufficiently inactivated at 4° C.

### Total Chemical Synthesis of Fibronectin Type I Domains

Total chemical synthesis by solid phase peptide synthesis (SPPS) and native chemical ligation of the F domains of hepatocyte growth factor activator (HGFA, SwissProt entry Q04756) and tPA (SwissProt entry P00750) was performed in the laboratory of Dr. T. M. Hackeng (Academic Hospital Maastricht, The Netherlands), according to described procedures known to a person skilled in the art. After synthesis of the peptide, the Fmoc group of the lysine can be selectively removed and biotinylated to eventually give finger-biotin²⁰⁰⁻²⁴⁰ in order to allow immobilization of finger on Streptavidin-coated surfaces. In addition, fluorescent labels can be attached. Previously, due to the length of the module and the high content of β-sheet structure, finger could only be successfully synthesized from two pieces that were joined by means of Native Chemical Ligation (NCL). The ligation process involves the chemoselective reaction between a C-terminal thioester and an N-terminal cysteine residue, resulting in a native peptide bond at the site of ligation (Dawson, 1994; Dawson 2000; Hackeng, 1999). To ensure correct disulfide bond formation, a two-step folding procedure was applied in which first, the unprotected cysteines were oxidized and second, the acetamidomethyl (Acm)-protected cysteines after treatment with iodine. A correctly folded bioactive finger domain that binds to crossbeta structures was obtained. The HGFA-F domain was supplied with an acetylated lysine residue. Products were analyzed on a reversed phase HPLC column and with mass spectrometry.

### IgIV

Human broad spectrum immunoglobulin G (IgG) antibodies, referred to as “intravenous Ig” (“IVIg” or “IgIV”), “gammaglobulin,” “intravenous immune globulin,” “intravenous immunoglobuiin” or otherwise, were obtained from the local University Medical Center Utrecht pharmacy department. Octagam from Octapharma (Octapharma International Services N.V., Brussels, Belgium), dosage 2.5 gr. in 50 ml, lot 4270568431, exp. 05-2006, hereinafter referred to as IgIV was used. Octagam is supplied as a ready-to-use solution comprising 50 mg/ml IgIV. Other components are 100 mg/ml maltose and less than 5 μg/ml, Triton X-100 and less than 1 μg/ml tri-n-butyl phosphate. It is stored at 4° C. According to the manufacturer, Octagam mainly consists of IgGs (≧95%), with a minor IgA fraction (≦5%). The distribution over the four IgG isotypes is: IgG1, 62.6%; IgG2, 30.1%; IgG3, 6.1%; IgG4, 1.2%. Octagam is preferably used at room temperature, and at 37° C. in several bioassays. Solutions were kept at room temperature for at least 30 minutes before use.

Culturing of Staphylococcus aureus Newman and Escherichia coli TOP10, and Preparing Reference and Tester Cell Samples.

Staphylococcus aureus Newman, which was a kind gift of Dr. Jos van Strijp and Dr. Kok van Kessel (Dept. of Microbiology, University Medical Center Utrecht, The Netherlands), was plated on a blood plate from a stock stored at −70° C., and incubated overnight at 37° C. The plate was stored at 4° C. Escherichia coli strain TOP10 (Invitrogen, 44-0301) was plated on agar with Luria broth medium from a −80° C. glycerol stock, and incubated overnight at 37° C. The plate was stored at 4° C. Overnight cultures of 5 ml in Luria broth medium were grown at 37° C. with vigorous shaking and aeration, by streaking a single colony with the tip of a pipette and transferring the tip to the medium in a 15-ml tube. The cell density in overnight cultures was determined by measuring the absorbance at 600 nm (A₆₀₀=1 is equivalent with 0.8×10⁹ cells/ml). Cells were pelleted by centrifugation for 1 minute at 16,000*g or for 10 minutes at 3,000*g. Medium was discarded. One half of the cells were resuspended in PBS in 1/10 of the original medium volume (10× concentration of the cells) by pipetting and swirling. These cells were used as reference cells. The second half of the cells was designated as “tester” cells and was resuspended in PBS with 5 mM Thioflavin T (again 1/10 of the original medium volume), by pipetting and swirling, as with all subsequent handlings. The tester cells were incubated for five minutes at room temperature with constant swirling. Cells were pelleted by centrifugation for 30 seconds at 16,000*g and supernatant was discarded. Cells were resuspended in 5 mM Congo red in PBS and incubated in a way similar to the Thioflavin T incubation. After pelleting the cells, they were resuspended in PBS and again pelleted by centrifugation for 30 seconds at 16,000*g. Supernatant was discarded. Finally, tester cells were resuspended in a solution of 25 μM tissue-type plasminogen activator (Actilyse, Boehringer-Ingelheim) and 25 mg/ml intravenous immunoglobulins (IgIV, Octagam, Octapharma), in approximately 1/150 of the original medium volume (75× concentration of the cells). After a 30-minute incubation, cells were pelleted, resuspended in PBS (approximately 1/10 of the original medium volume) and kept at room temperature for use at the same day or kept at 4° C. for later use within 72 hours. E. coli cell suspension was yellow-light orange after all subsequent incubations, S. aureus cell suspension was red. Initial cell densities of the overnight cultures were 1.8×10⁹ cells/ml for the S. aureus and 1.3×10⁹ for the E. coli cells. The work suspensions had cell densities of 1.8×10¹⁰ cells/ml and 1.8×10¹⁰ cells/ml, respectively.

Reference and tester Staphylococcus aureus Newman cells or Escherichia coli TOP10 cells obtained as described above were applied in a series of bioassays. Cells at various indicated densities were analyzed for their activity with respect to i) ROS production in murine ECs, ii) platelet aggregation, iii) plasma coagulation in PT and aPTT analyses, and iv) tissue factor expression in THP-1 monocytes. In addition, plasmin generation in a chromogenic tPA/plasminogen activation assay is assessed and activation of factor XII and prekallikrein is assessed in a chromogenic assay. For this purpose, dilution series of the pathogen cells are either mixed with final concentrations of 400 pM tPA, 0.2 μM plasminogen (purified from human plasma) and 0.8 mM chromogenic plasmin substrate S2251 (Chromogenix) or chromogenic plasmin substrate Biopep-1751 (Biopep, France) in a physiological buffer, or with 0.3 mM chromogenic kallikrein substrate Chromozym-PK (Roche Diagnostics, Almere, The Netherlands), 1 μg/ml zymogen factor XII (#233490, Calbiochem, EMD Biosciences, Inc., San Diego, Calif.), human plasma prekallikrein (#529583, Calbiochem) and human plasma cofactor high-molecular weight kininogen (#422686, Calbiochem). For the factor XII assay, the assay buffer contained HBS (10 mM HEPES, 4 mM KCl, 137 mM NaCl, pH 7.2). Plasmin or kallikrein generation is followed in time upon 37° C. incubation by measuring the A₄₀₅ absorbance each minute for two to three hours. Buffer serves as a negative control, concentration series of glycated hemoglobin and/or of amyloid γ-globulins, prepared as described above, and/or 150 μg/ml kaolin serve as positive controls.

Production of Reactive Oxygen Species by Mouse Microvascular bEnd.3 Endothelial Cells

To assess production of reactive oxygen species by cultured mouse microvascular bEnd.3′ endothelial cells (ECs), cells are seeded at 128,000 cells/well of a 96-well plate (Costar, 3904). After adherence for six hours, cells are washed twice with PBS and cultured overnight in DMEM with 0.1% bovine serum albumin (DMEM from Gibco with 4500 mg/l glucose, GlutaMAX and pyruvate, enriched with 100 μg/ml penicillin and streptomycin and 10% fetal calf serum). Cells are subsequently washed once with PBS enriched with 1 mM CaCl₂, 0.5 mM MgCl₂ and 0.1% w/v glucose (“enriched PBS”) and incubated for 30 minutes at 37° C. in the dark with 75 μl of CM-H2DCFDA (Invitrogen C6827) from a 10 μM stock in PBS. Then, cells are washed twice with enriched PBS and incubated for 15 minutes at 37° C. in the dark, either with 190 μl enriched PBS, or 190 μl enriched PBS with 1 μM Nω-Nitro-L-arginine methyl ester hydrochloride. For the analysis of ROS production, 10 μl of tester samples and controls are added to separate wells, and fluorescence is measured every two minutes for 70 minutes upon excitation at 488 nm with the emission wavelength set to 538 nm.

In one series of experiments, bEnd.3 cells were exposed to 160× diluted stocks of E. coli TOP10 or S. aureus (final cell densities 8.1×10⁷ cells/ml and 1.13×10⁸ cells/ml, respectively) in buffer or in buffer with either 1.25 mg/ml IgIV (Octagam), or finger domains (see below), or 220 μM Congo red, or 220 μM Thioflavin T (ThT), or 1.1 μM tPA, and ROS levels were followed in time upon 37° C. incubation. The bacteria were pre-incubated with PBS or the crossbeta structure-binding compounds at concentrations of 25 mg/ml IgIV, 0.8 mg/ml finger domains, 4.4 mM Congo red, 4.4 mM Thioflavin T, or 22 μM tPA, respectively, for approximately one hour at room temperature. Subsequently, the bacterial cell suspensions were diluted twenty-fold in the cell culture medium with the ECs. As a source of finger domains, a mixture was prepared consisting of recombinant human tPA finger (F) with a C-terminal His-tag, which was expressed in Saccharomyces cerevisiae (Biotechnology Application Center (BAC-Vlaardingen/Naarden, The Netherlands)), a chemically synthesized hepatocyte growth factor activator (HGFA) finger domain (Dr. T. Hackeng, Academic Hospital Maastricht, The Netherlands) and recombinant human fibronectin finger domain tandem 4 and 5 with a C-terminal His-tag, which was expressed in HEK 293E cells (ABC-Expression Facility, Utrecht). The cDNA constructs were prepared following standard procedures known to a person skilled in the art, as described above. S. A. Newman (New York Medical College, Valhalla, USA) and A. Muro (ICGEB, Trieste, Italy) kindly provided the cDNA encoding for HGFA, for an N-terminal fragment of human fibronectin, comprising fibronectin type I domains 4-5, and for a C-terminal fibronectin fragment, comprising fibronectin type I domains 10-12, respectively. Domain boundaries of fibronectin F4-5 and tPA-F were taken from the human fibronectin and human tPA entries in the Swiss-Prot database (P02751 for fibronectin, P00750 for tPA) and comprised amino acids NH₂-I182-V276-COOH of fibronectin and NH₂-G33-S85-COOH of tPA. Affinity purification of the expressed proteins was performed using His₆-tag-Ni²⁺ interaction and a desalting step. For HGFA, residues 200 to 240 (Swiss-Prot entry Q04756) were taken. Stock solutions of fibronectin. F4-5, tPA-F and HGFA-F were mixed to final concentrations of 0.9 mg/ml, 0.7 mg/ml and 1.25 mg/ml, respectively. The final concentration of finger domains is approximately 0.8 mg/ml.

### Fibrin Clot Lysis Assay

Fibrin clots were prepared from normal pooled citrated plasma (freshly frozen normal pool of healthy volunteers) in wells of an ELISA plate (Costar, catalogue number 2595, Cambridge, Mass., USA). Fifty μl of plasma was mixed 1(:)1 with 50 μl reaction mix containing phosphatidylcholine/phosphatidylserine vesicles at 8 and 32 μM, from a stock of 0.36/1.43 mM in 25 mM Tris-HCl, 150 mM NaCl pH 7.3, 16.6 mM CaCl₂. 10 μl of a 10× HBS stock (1×HBS is 10 mM HEPES, 4 mM KCl, 137 mM NaCl, pH 7.2) and 10 μl tissue factor (Innovin, catalogue number B4212-50, Dade Behring Marburg GmbH, D-35041 Marburg, Germany). Clotting was allowed to proceed during a 10- to 15-minute incubation at 37° C. Subsequently, 50 μl with 1 μM tPA or K2P-tPA in HBS was added and lysis at 37° C. was followed in time by reading the absorbance, each 30 seconds at 405 nm on a spectrophotometer (Spectramax, Molecular Devices Ltd, Wokingham, England). To test the influence of sRAGE on lysis efficiency, sRAGE was added to the tPA solution. In control wells, tPA or K2P-tPA was omitted from the solutions. Background signals obtained with these wells were subtracted. Samples were measured in six-fold and averaged. Lysis by tPA was set at 100%.

Induction of Platelet Aggregation by Crossbeta Structure and Pathogens with Crossbeta Structure

The influence of proteins comprising crossbeta structure, S. aureus Newman bacterium cells and E. coli TOP10 bacterium cells on blood platelet aggregation was tested with washed platelets (platelet-rich plasma, PRP) in an aggregometric assay. Freshly drawn human aspirin-free blood was mixed gently with citrate buffer to avoid coagulation. Blood was spinned for 15 minutes at 150*g at 20° C. and supernatant was collected; platelet-rich plasma (PRP) with an adjusted final platelet number of 300,000 platelets/μl. Platelets were kept at 37° C. for at least 30 minutes before use in the assays to ensure that they were in the resting state. Platelets of two donors were isolated separately on different days.

For the aggregometric assays, 270 μl platelet solution was added to a glass tube and prewarmed to 37° C. A stirring magnet was added and rotation was set to 900 rpm, and the apparatus (Whole-blood aggregometer, Chrono-log, Havertown, Pa., USA) was blanked. A final volume of 30 μl was added, containing the agonist of interest (pathogen) and/or the premixed antagonist of interest (pathogen pretreated with crossbeta structure-binding molecules), prediluted in HEPES-Tyrode buffer pH 7.2. Final S. aureus concentration was 1.8×10⁹ cells/ml; for E. coli 1.3×10⁹ cells/ml. Aggregation was followed in time by measuring the absorbance of the solution that will decrease in time upon platelet aggregation. As a positive control, 5 μM of synthetic thrombin receptor-activating peptide TRAP was used. Aggregation was recorded for 15 minutes and expressed as the percentage of the transmitted light (0-100%). For experiments with indomethacin, AR-C69931MX and iloprost, the platelets were preincubated with 30 μM and 50 nM and 20 ng/ml antagonists for 30, two, and two minutes (respectively) before aggregation.

Activation of the Contact System of Coagulation by E. coli with Amyloid Curli

E. coli strain MC4100 was grown using two different conditions on agar with colonization stimulating factor (CFA), using protocols known to a person skilled in the art. E. coli on one plate were grown for approximately 44 hours at 26° C. to induce expression of amyloid curli core protein comprising crossbeta structure. A second plate was cultured for 24 hours at 37° C., which suppresses curli expression. Cells were scraped from the plates and suspended in PBS. Cell density was measured and equalized. The two E. coli preparations were tested for their ability to activate factor XII and prekallikrein in an in vitro assay for determination of contact system of coagulation-activating properties. For this purpose, an E. coli density of 2.08×10⁹ cells/ml was used in the assay that was performed as described above.

Analysis of Tissue Factor Expression by THP-1 Upon Stimulation with S. aureus that were Pre-Incubated with Buffer or Crossbeta Structure-Binding Compounds

For tissue factor expression analysis purposes, THP-1 cells were cultured in IMDM without gentamycin and streptomycin. At day 0, one ml of cells was seeded at 1×10⁶ cells/ml in the wells of six-well culture plates. At day 1, cells were stimulated for six hours at 37° C. with S. aureus that were pre-incubated with PBS or with crossbeta structure-binding compounds ThT, Congo red, tPA and IgIV, as described above, at a cell density of 1.8×10⁷/ml (regular culturing conditions). Negative control was buffer. After six hours, the cells were pelleted by centrifugation and resuspended in 100 μl TBS (50 mM Tris-HCl, 150 mM NaCl, pH 7.0-7.3). Next, the cells were frozen and thawed for four subsequent cycles. Cells were centrifuged for ten minutes at 16,000*g and the supernatant was used for analysis of tissue factor (TF) expression. First, protein concentrations were determined using an established protein concentration assay (Bicinchoninic Acid (BCA) Protein Assay). Protein concentrations were equalized between samples with TBS to correct for variations in cell density. For analysis of TF levels, 50 μl of cell lysate was mixed with 50 μl TBS comprising 10 μg/ml factor X, 5 U/ml recombinant activated factor VII (rFVIIa, Novoseven, NovoNordisk, left-over vial that returned from the clinic and that was no longer suitable for human use) and 5 mM CaCl₂, and 50 μl of a 4.5 mM stock of chromogenic activated factor X substrate S2765 in H₂O, in wells of a 96-well plate. Conversion of the substrate by activated factor X at 37° C. was recorded in time for 100 minutes, by absorbance readings at 405 nm. As an additional control, factor X activity was assessed with S. aureus cells only, omitting the monocytes.

In a second series of experiments, THP-1 monocytes were incubated in a similar manner with 40 μg/ml glycated hemoglobin or 10 μg/ml amyloid-β(1-40). Negative control was buffer, positive control was 10 μg/ml lipopolysaccharide.

### Example 29

### Binding of Anti-β2gpi Autoantibodies is Inhibited by tPA

Auto-antigen human β2-glycoprotein I (β2gpi) purified from plasma with a monoclonal antibody column was immobilized on a high-absorbing ELISA plate. It is commonly assumed that the auto-antigen denatures at this surface. Binding of purified human IgG auto-antibodies against β2gpi was determined by incubating 100 μg/ml antibody in the wells of the ELISA plate with β2gpi. The purified auto-antibodies were pre-incubated with a concentration series of tPA, before mixtures were added to the wells. tPA was used at concentrations ranging from 0 to 500 nM. Binding of the auto-antibodies was assessed with anti-human IgG antibody with alkaline-phosphatase.

### Example 30

### Materials and Methods of Mouse Tail Bleeding Assay

For the analysis of the influence of crossbeta structure-binding compounds on in vivo coagulation and/or platelet aggregation, the mouse tail cut assay was performed to determine bleeding time. For this approach, fifty 11- to 13-week-old male black six C57BL/6JOlaHsd mice were used according to a protocol that was approved by the local ethical committee for animal experiments (Utrecht University, The Netherlands). Mice were injected intravenously (i.v.) with 100 μl buffer (PBS) or buffer with compound. In the control group (n=14), PBS was injected i.v. in the tail vein. After five to twenty minutes, the mice were anesthetized in a chamber with 5% Isofluoran (induction), followed by anesthesia with 2-2.5% Isofluoran using a mask during the course of the experiment (maintenance). Mice were kept at a warmed blanket (37° C.) with their tail hanging off the table. Five mm was cut off the tail with a scissors and blood was collected in cups. Time between injection and the tail cut was recorded, as well as the time between the start of bleeding and when bleeding (was) stopped. End points were stop of bleeding, bleeding time lasting longer than 20 minutes, which was actively stopped by burning, and reaching a bled volume of over 200 μl due to fast bleeding. Prolonged bleeding for over 20 minutes and relatively excessive bleeding were both set to a bleeding time of 20 minutes. As a positive control for expected prolonged bleeding, 10 I.E./mouse heparin (Leo Pharmaceutical Products B.V., 5000 IE/ml) i.v. in 100 μl 0.9% NaCl (n=8) were used. Hepatocyte growth factor activator (HGFA) finger domain or fibronectin type I domain was used at 4.7 mg/ml. One hundred μl was injected i.v., resulting in an approximate final concentration of 234 μg/ml based on an estimated blood volume of 2 ml/mouse (n=14). Human intravenous immunoglobulins (Octagam, OctaPharmia) from a 50 mg/ml stock as supplied by the manufacturer were used 20 times diluted (n=14).

A chemically synthesized hepatocyte growth factor activator (HGFA) finger domain was synthesized (Dr T. Hackeng, Academic Hospital Maastricht, The Netherlands). For HGFA, residues 200 to 240 (Swiss-Prot entry Q04756) were taken.

### Results

### Example 19

### In Vitro Murine bEend.3 Endothelial Cell Activation Assay: ROS Production (I)

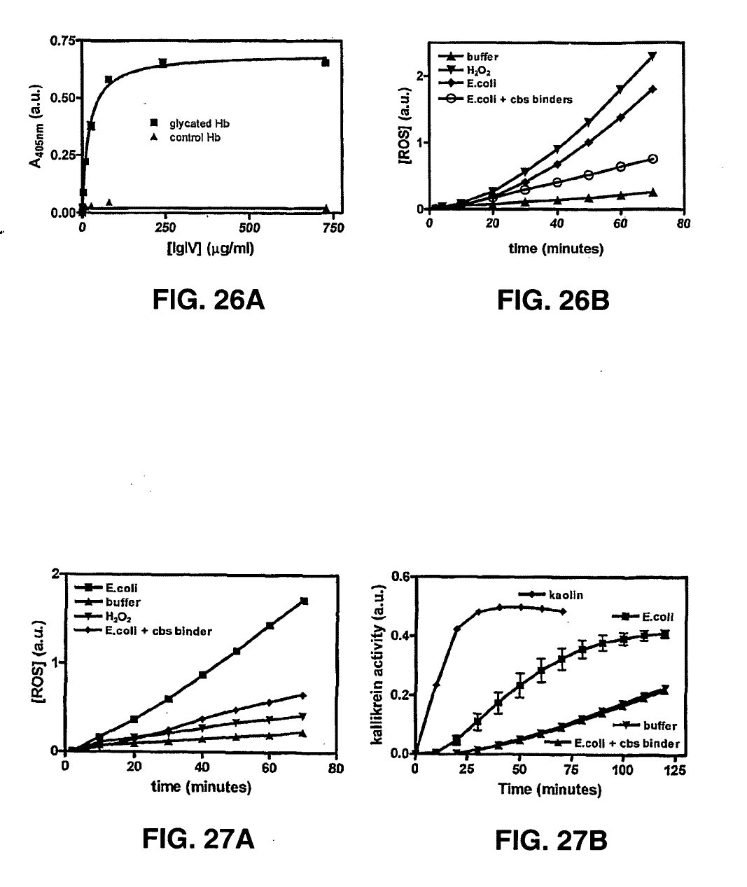

To determine whether crossbeta structure-binding compounds Thioflavin T, Congo red, tissue-type plasminogen activator (tPA) and IgIV are able to reverse adverse effects of pathogens on ECs, bEnd.3 cells were exposed to 6×10⁸ E. coli TOP10 cells/ml and ROS production by the ECs was measured in time. For this purpose, bEnd.3 cells were cultured overnight at a density of 128,000 cells/well of a 96-well plate. The overnight grown E. coli cells were either resuspended in PBS before 20× dilution in cell culture medium at 1.2×10⁹ cells/ml (20× stock), or resuspended in 2.5 mM. Congo red and 5 mM Thioflavin T in PBS after centrifugation and discarding the LB medium, incubated for ten minutes at room temperature with swirling, pelleted and dissolved at 1.2×10⁹ cells/ml (20× stock) in 25 μM tPA and 25 mg/ml IgIV by swirling. As an example, binding of IgIV to crossbeta structure is shown for glycated albumin (FIG. 26A). BEnd.3 cells were incubated with PBS or with 100 μM H₂O₂ as negative and positive control for ROS induction, respectively (FIG. 26B). FIG. 26B shows the increased ROS production when bEnd.3 cells are exposed to E. coli cells. Pre-incubation of E. coli cells with Congo red, Thioflavin T, tPA and IgIV reduces ROS production significantly (FIG. 26B). This inhibition of ROS production by bEnd.3 ECs upon exposure to E. coli cells that are pre-incubated with crossbeta structure-binding compounds show that the crossbeta structure at the surface of the E. coli cells mediate pathogenic effects on ECs.

In a second series of experiments, E. coli TOP10 cells were pre-incubated in a serial set-up with PBS comprising first, 2.5 mM Thioflavin T, then 5 mM Congo red and finally, a mixture of 25 μM tPA and 25 mg/ml IgIV, respectively. Control cells were kept in PBS. Finally, E. coli cells were resuspended in PBS at 1.3×10¹⁰ cells/ml. Notably, pelleted cells appeared brownish-yellow and the cell suspension was orange-yellow after the incubations with crossbeta structure-binding compounds, whereas the control cells in PBS were light brown. To determine the influence of the two E. coli preparations on ROS production by bEnd.3′ ECs, 8×10⁷ E. coli cells/ml were exposed to the ECs. In FIG. 27A, increased ROS production upon stimulation of ECs with the control E. coli cells is observed. An excess of vascular ROS flux correlates with the onset of atherothrombosis. ROS induce oxidant stress that promotes EC malfunctioning, causes oxidative injury to vascular cells, oxidizes lipoproteins and accelerates atherothrombogenesis. Pre-treatment of the E. coli with crossbeta structure-binding compounds decreases the potency to induce ROS production (FIG. 27A).

### Example 20

### Factor XII/Prekallikrein Activation by E. Coli

To test the potency of E. coli cells to induce factor XII/prekallikrein activation, cells at 1.3×10⁷ cells/ml were tested in a chromogenic factor XII activation assay using chromogenic kallikrein substrate Chromozym-PK. Activation of factor XII to factor XIIa and subsequently prekallikrein to kallikrein is observed upon incubation of the proteins with E. coli cells (FIG. 27B). Pre-treatment of the cells with PBS containing first, 5 mM Thioflavin T, then 5 mM Congo red and finally, a mixture of 25 μM tPA and 25 mg/ml IgIV, resulted in background factor XII activation, similar to when buffer is used as a negative control. These results show that incubation of E. coli with crossbeta structure-binding compounds reduces the potency to activate the intrinsic pathway of coagulation in hemostasis, thereby preventing a pathogen-induced procoagulant state.

### Example 21

### Induction of Platelet Aggregation by Cross Beta Structures on Pathogens

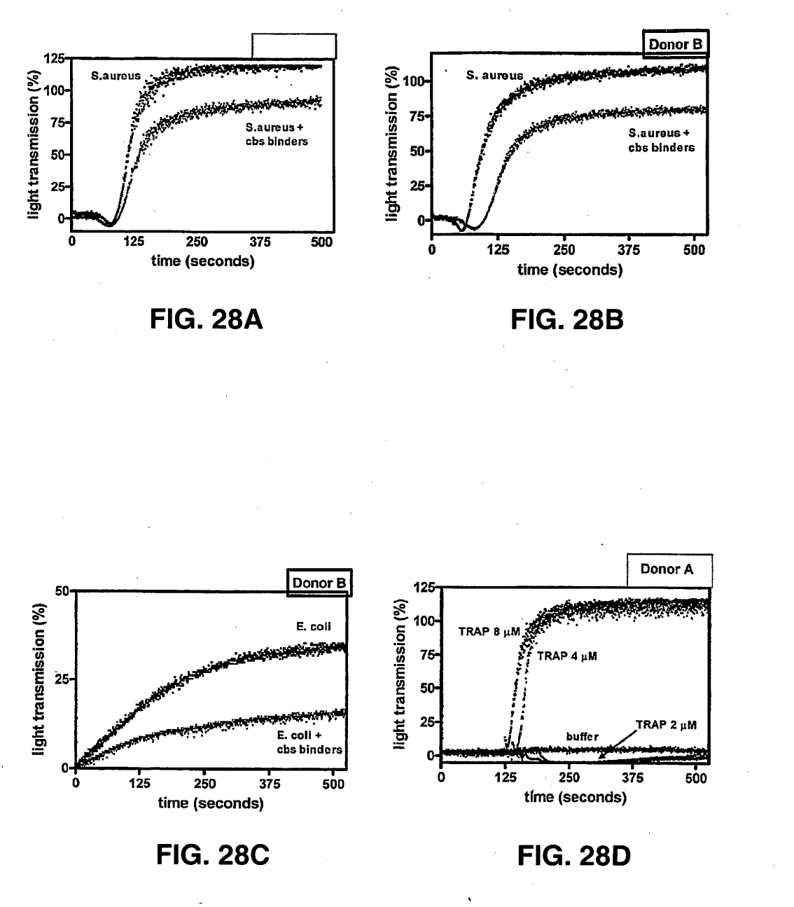

PRP was obtained from blood obtained from the local UMC Utrecht mini donor facility. Introduction of 1.8×10⁹ S. aureus Newman cells/ml or 1.3×10⁹ E. coli TOP10 cells/ml in PRP readily results in platelet aggregation (FIG. 28). The S. aureus is a more potent stimulator of platelet aggregation than the E. coli strain, when similar cell densities are tested. Both PRP of donors A and B respond similarly to S. aureus, whereas only PRP obtained from donor A was activated by E. coli. Pre-treatment of the S. aureus cells and the E. coli cells with, respectively, 2.5 mM Thioflavin T, 5 mM Congo red, and 25 μM tPA+25 mg/ml IgIV inhibits pathogen-induced cell aggregation (FIG. 28). These data show that surface-exposed cross-beta structures induce platelet aggregation. Binding of a crossbeta structure-binding compound to the surface-exposed cross-beta structures counteracts cross-beta structure-induced platelet aggregation.

### Example 22

### Binding of Crossbeta Structure-Binding Compounds to E. coli TOP10 and S. aureus Newman

Binding of crossbeta structure-binding compounds to E. coli TOP10 and S. aureus Newman after incubation of the bacteria with Thioflavin T, Congo red, tPA and IgIV, as described in the Materials and Methods section, was determined in two ways. First, binding of crossbeta structure-binding dyes Thioflavin T (yellow) and Congo red (red) to the bacterium, cells was verified by visual inspection. The E. coli appeared as yellowish-orange cells, showing that Thioflavin T was bound to the cells and to a lesser extent Congo red. The S. aureus cells were intense red, indicative for Congo red binding. Due to the intense red color, yellow Thioflavin T could not be seen. In conclusion, S. aureus binds more Congo red than E. coli, whereas, no comparative qualitative measure can be given for Thioflavin T binding. Obviously, Thioflavin T is bound to E. coli.

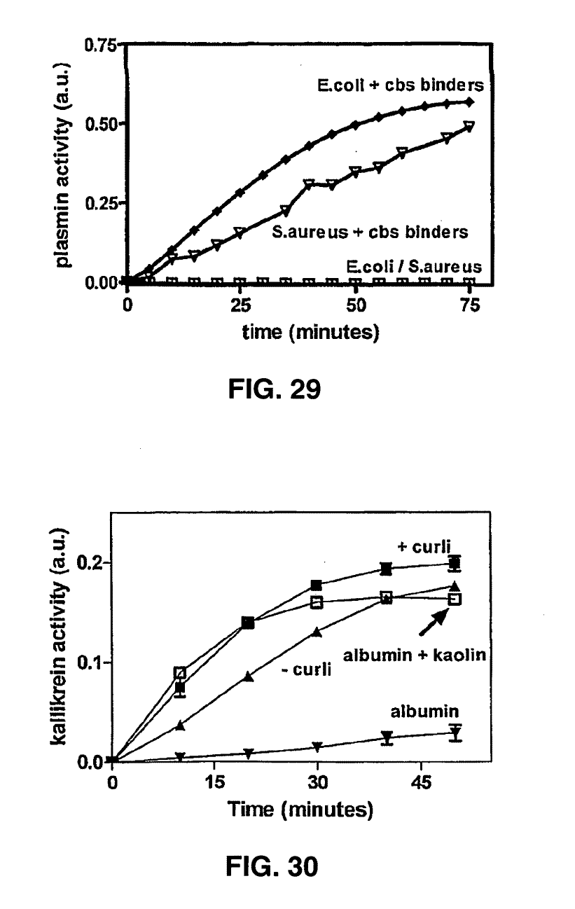

Whether tPA is bound to the E. coli and S. aureus after incubation with 25 μM tPA, was assessed with a tPA/plasminogen chromogenic activation assay, as described above. Plasminogen and chromogenic plasmin substrate Biopep-1751 were mixed with E. coli or S. aureus incubated with buffer only, or with E. coli or S. aureus that were pre-incubated with, amongst other crossbeta structure-binding compounds, tPA. Plasmin generation by tPA, measured as conversion of the substrate, only occurs when an external source of tPA activity is introduced in the reaction mixture (FIG. 29).

### Example 23

### Activation of the Contact System of Blood Coagulation by E. coli with Amyloid Curli Protein

It has been established that E. coli bacteria express an amyloid core protein, curli, at the cell surface, depending on culturing conditions. When E. coli MC4100 is cultured on CFA agar for 44 hours at 26° C., expression of curli is facilitated, whereas, no curli is expressed when cells are grown for 24 hours at 37° C. Curli with crossbeta structure are an important determinant for binding properties of the E. coli towards fibronectin of the host, the fibronectin being a crossbeta structure-binding protein through the ability of the finger domains (fibronectin type I domains) 4, 5, 10, 11 and 12, which are capable of binding to proteins comprising crossbeta structure. The E. coli with and without amyloid curli comprising crossbeta structure were applied to a chromogenic factor XII/prekallikrein activation assay. When factor XII becomes activated, kallikrein is formed from prekallikrein by activated factor XII, and kallikrein substrate Chromozym-PK is converted, which is measured by absorbance readings at 405 nm in time. In FIG. 30, it is shown that E. coli with curli is a more potent activator of the contact system of coagulation than E. coli lacking−curli. These observations show that amyloid curli are involved in activating the contact system.

### Example 24

### In Vitro Murine bEend.3 Endothelial Cell Activation Assay: ROS Production (II)

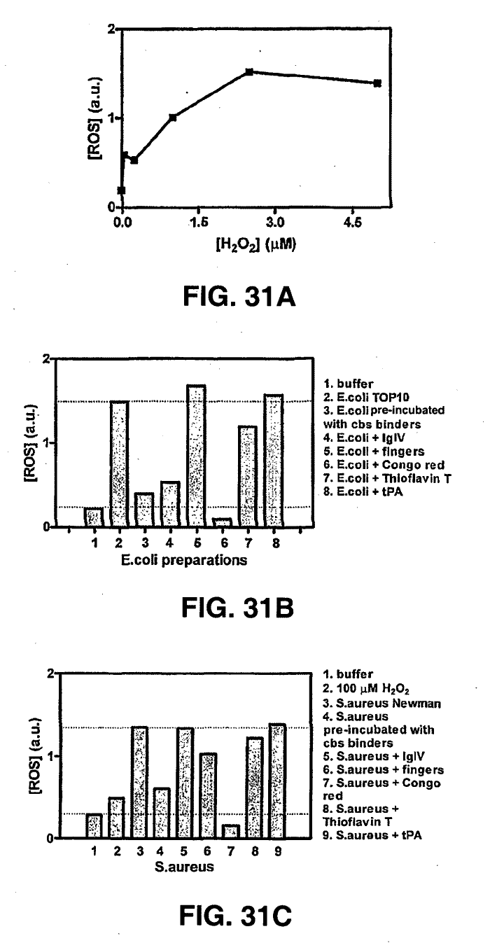

To test whether crossbeta structure-binding compounds ThT, Congo red, IgIV, tPA and finger domains of fibronectin, HGFA and tPA have the potency to reverse adverse effects of pathogens E. coli TOP10 and S. aureus Newman on bEnd.3 ECs with respect to ROS expression, the ECs were exposed to 8.1×10⁷ E. coli cells/ml or 1.13×10⁸ S. aureus cells/ml in the presence of buffer, or in the presence of either 1.25 mg/ml IgIV, or 0.8 mg/ml finger domains, or 220 μM Congo red, or 220 μM ThT, or 1.1 μM tPA. ROS levels were followed in time upon 37° C. incubation. The bacteria were also pre-incubated with ThT, Congo red, IgIV and tPA as described above. From FIG. 31B, it is clear that pre-incubation of E. coli with crossbeta structure-binding compounds counteracts proatherogenic effects of the bacterium towards ECs. Co-incubations of E. coli with IgIV, Congo red and, to some extent, ThT, also reverse ROS production. Finger domains and tPA at the conditions tested are not able to reduce ROS production. For S. aureus, also pre-incubation of the bacterium with Congo red, ThT, tPA and IgIV reduced ROS production by the bEnd.3 cells (FIG. 31(C). In addition, co-incubations of bEnd.3 with S. aureus and Congo red also strongly inhibits ROS expression, whereas finger domains and ThT at the conditions tested, slightly decrease ROS production. In contrast to E. coli, in this set-up, IgIV does not influence S. aureus-induced ROS production. Also, tPA does not influence S. aureus-induced ROS production in this experimental set-up. Further studies will include refinements of the assay conditions with respect to dosing, experimental settings like buffer, excipients, assay time, cell densities and more. In summary, it is concluded that crossbeta structure-binding compounds like, for example, ThT, Congo red, finger domains and IgIV are able to reverse adverse effects of pathogens on ECs.

### Example 25

### Ex Vivo Human Plasma Coagulation Assays

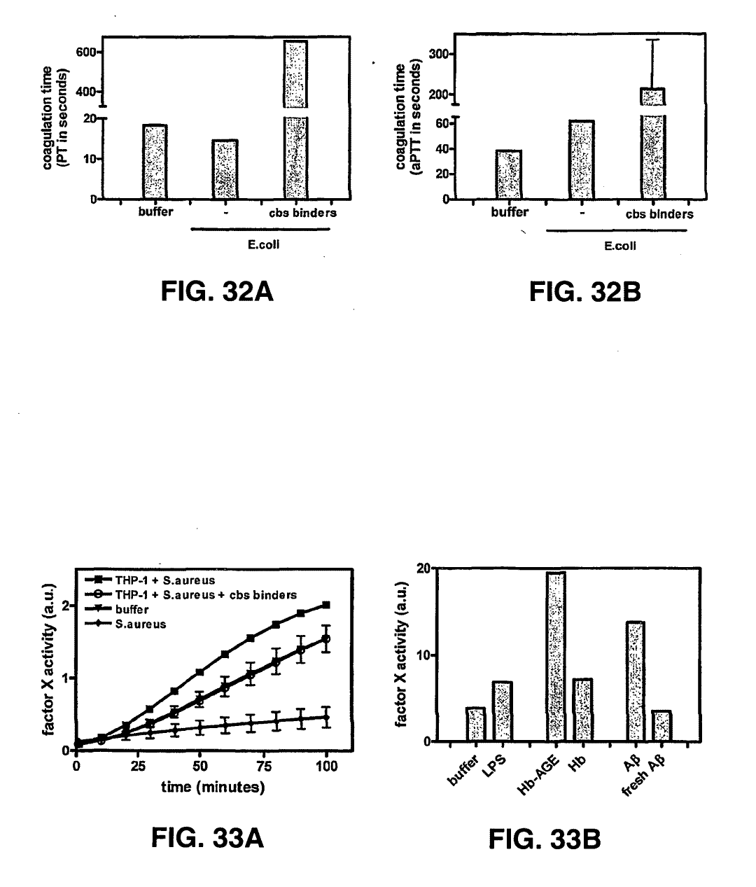

For analysis of the influence of crossbeta structure-comprising pathogens on the characteristics of blood coagulation, and for analysis of the effects of crossbeta structure-binding compounds on the influence of pathogens on coagulation, aPTT and PT coagulation tests were performed. Pooled human plasma of approximately 40 apparently healthy donors was clotted by adding either negatively charged phospholipids, CaCl₂ and kaolin in the aPTT set-up, or tissue factor-rich thromboplastin and CaCl₂ in the PT set-up. Before coagulation tests were performed, two-fold diluted plasma was pre-incubated for approximately one hour at room temperature with PBS (control), 6.5×10⁹ E. coli TOP10 cells/ml, or 6.5×10⁹ E. coli TOP10 cells/ml that were pre-incubated with crossbeta structure-binding compounds Congo red, ThT, tPA and IgIV. Before coagulation tests were performed, bacterium cells were pelleted by centrifugation and plasma supernatants were analyzed in the aPTT and PT assays. Results are shown in FIG. 32. The pre-incubation of plasma with E. coli results in acceleration of coagulation with approximately 20% in a PT, and a delayed coagulation with approximately 60% in an aPTT (FIG. 32). Pre-incubations of E. coli with crossbeta structure-binding compounds results in a strongly delayed coagulation in both tests. In the PT analyses, measurements last for up to 650 seconds and were stopped when no coagulation was observed at that time. Similarly, aPTT analyses were stopped after approximately 330 seconds when no coagulation had occurred.

When E. coli are pre-treated with crossbeta structure-binding compounds, coagulation is strongly delayed in both PT and aPTT analyses (FIG. 32). This shows that the crossbeta structure-binding compounds are bound to the E. coli cells. Without wishing to be bound to theory, it is thought that the strongly delayed coagulation is related to bound tPA at the E. coli surface, which can generate plasmin from plasminogen when a suitable crossbeta structure cofactor is present at the E. coli surface. In the coagulation tests, the plasmin will dissolve fibrin clots that are formed. Apparently, fibrinolytic activity is so strong that a formed clot is again readily lysed or not formed at all.

### Example 26

### Analysis of Tissue-Factor Expression by THP-1 Upon Stimulation with S. Aureus that were Pre-Incubated with Buffer or Crossbeta Structure-Binding Compounds

For tissue factor expression analysis purposes, THP-1 cells were exposed to 1.8×10⁷ S. aureus cells/ml for six minutes at 37° C. TF activity was determined in an indirect way by assessing activation of factor X in the presence of activated factor VII, with three-fold diluted THP-1 monocyte cell lysate. Factor X activity in THP-1 cell lysates after exposure to PBS-incubated S. aureus was higher than in lysates of cells that were exposed to S. aureus, which was pre-incubated with crossbeta structure-binding compounds Thioflavin T, Congo red, tPA and IgIV (FIG. 33A). Pre-incubation of S. aureus with the crossbeta structure-binding compounds results in return to basal TF activity levels seen with unstimulated THP-1 cells (FIG. 33A). These results show that proteins with crossbeta structure at the core of the pathogen induce a procoagulant state of the monocytes by TF up-regulation. Crossbeta structure-binding compounds effectively inhibit induction of this procoagulant state.

Misfolded proteins with crossbeta structure conformation, i.e., glycated hemoglobin (Hb-advanced glycation end product, AGE) and amyloid-β induce significantly elevated levels of TF, as determined by potent factor X activation by THP-1 monocyte lysates after incubation of the cells with the misfolded proteins (FIG. 33B), as compared to freshly dissolved amyloid-β (Aβ), freshly dissolved hemoglobin (Hb) and 10 μg/ml lipopolysaccharide (LPS).

### Example 27

### Platelet Aggregation is Induced by Proteins Comprising Crossbeta Structure and Inhibited by Crossbeta Structure-Binding Molecules

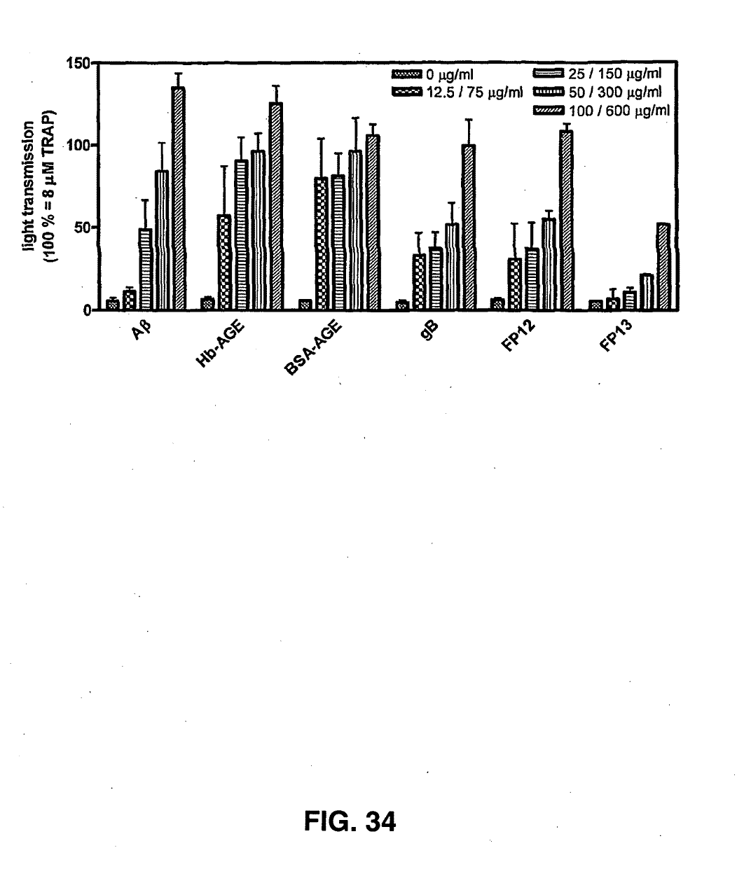

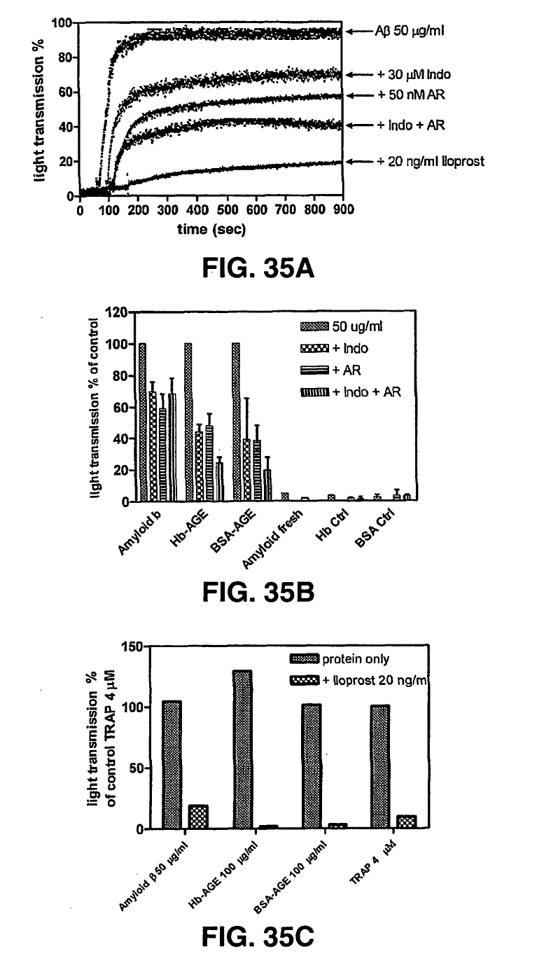

Platelet aggregations were performed as described above. Presence of crossbeta structure in peptides amyloid-β, Herpes simplex virus glycoprotein B peptide, fibrin α-chain peptide FP12, fibrin α-chain peptide FP13, glycated hemoglobin and glycated albumin was confirmed with ThT and Congo red binding assays and tPA/plasminogen activation assay, as well as by inspection under a transmission electron microscope. All proteins and peptides with crossbeta structure induce platelet aggregation in a dose-dependent manner (FIG. 34). These data show that platelets are stimulated by crossbeta structure in general. Both Indomethacin and AR-C69931MX inhibit the platelet aggregation to some extent, whereas Iloprost is fully capable of reversing the effects of misfolded protein on platelet aggregation (FIG. 35).

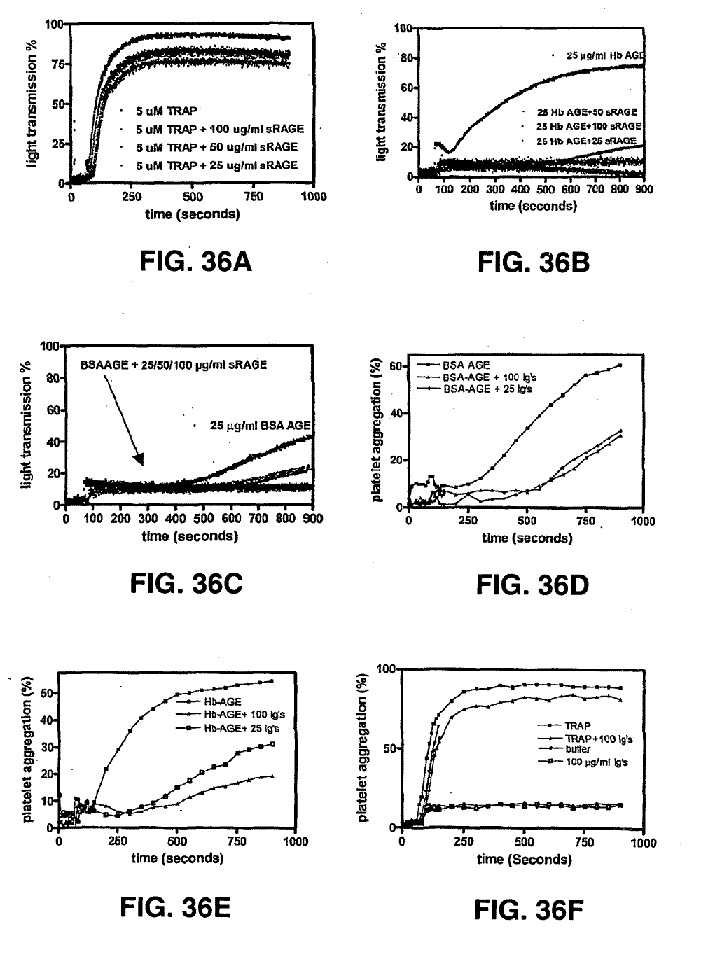

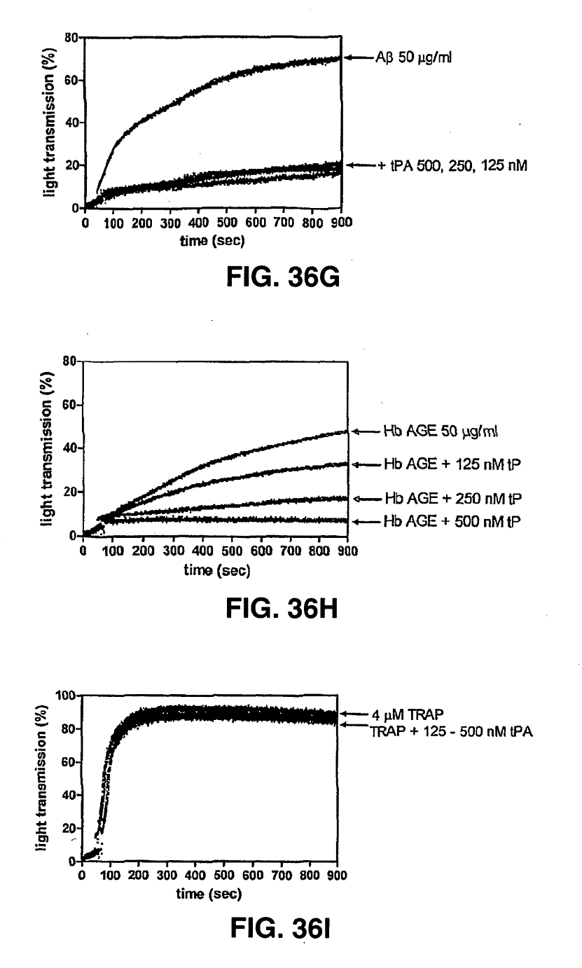

In FIG. 36, it is demonstrated that triggering platelet aggregation by glycated hemoglobin (Hb-AGE), glycated albumin (BSA-AGE) and amyloid-β(1-40) (Aβ) is effectively blocked by crossbeta structure-binding compounds, including soluble fragment of receptor for advanced glycation end products (sRAGE), intravenous immunoglobulins (IgIV) and tPA. TRAP-induced platelet aggregation is barely influenced by these crossbeta structure-binding compounds, demonstrating that activation of platelets by crossbeta structures follows a distinct and unique mechanism. In conclusion, the data show that anti-coagulant and/or anti-platelet aggregation therapy based on molecules that are able to prevent crossbeta structure-induced platelet aggregation is possible.

### Example 28

### Crossbeta Structure-Binding Compounds Interfere with Blood Coagulation and Fibrinolysis

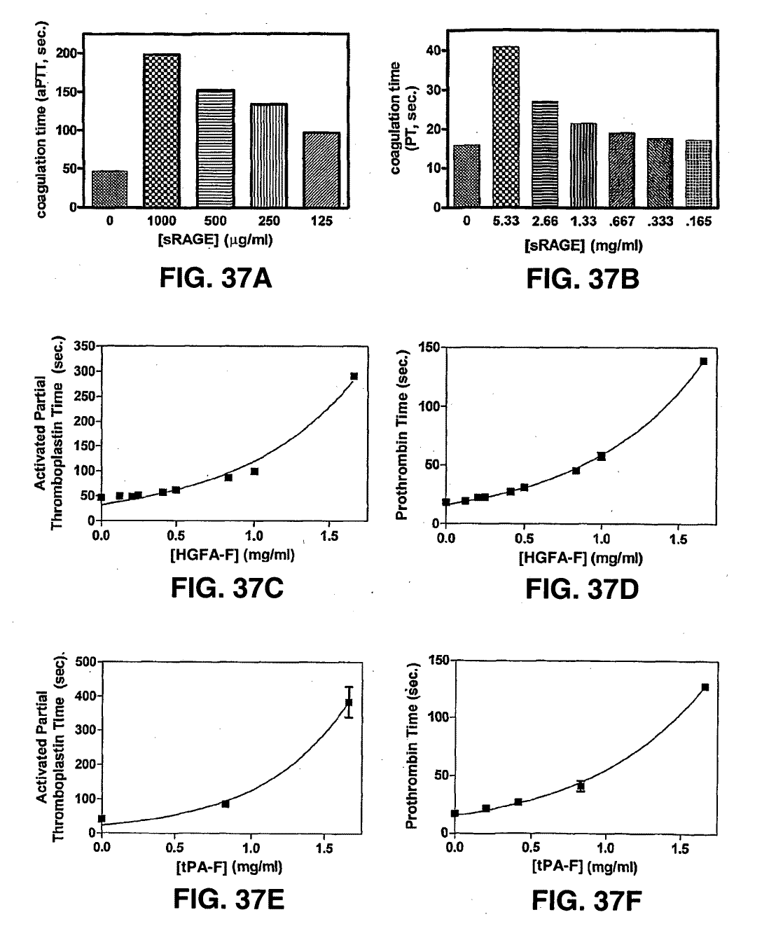

When applying concentration series of sRAGE or finger (F) domains of HGFA and tPA in PT and aPTT analyses, it was demonstrated that these crossbeta structure-binding compounds efficiently prolong coagulation times (FIG. 37). In PT analyses, the compounds provoke a direct effect on fibrin polymerization through formation of crossbeta structure, by inhibiting efficient polymerization. Without wishing to be bound to theory, it is thought that in aPTT set-ups, the crossbeta structure-binding compounds also counteract activation of factor XII by denatured proteins comprising crossbeta structure.

### Example 29

### Binding of Anti-β2-Glycoprotein I Autoantibodies from Patients Suffering from Anti-Phospholipid Syndrome to Denatured β2-Glycoprotein I Auto-Antigen is Strongly Diminished by tPA

It is well-known that auto-antibodies against self β2-glycoprotein I are directed against auto-antigen that is misfolded, and that the auto-antibodies do not bind to native protein. Auto-antibodies against β2-glycoprotein I (β2gpi) affinity purified from patients suffering from the Anti-Phospholipid Syndrome (APS) bind to β2gpi that is purified from human plasma and denatured at the surface of high-absorbing ELISA plates (see FIG. 39). Pre-incubation of the auto-antibodies with a concentration series of crossbeta structure-binding compound tissue-type plasminogen activator (tPA) resulted in strongly reduced binding of the auto-antibodies to immobilized denatured β2gpi auto-antigen. This shows that tPA binds to the denatured β2gpi and thereby competes with auto-antibodies for binding sites. This shows that binding sites for crossbeta structure-binding tPA and the auto-antibodies directed against denatured β2gpi overlap or are spatially closely oriented. This demonstrates a potential role of crossbeta structure in B2gpi in eliciting the auto-immune response in APS patients.

### Example 30

Averaged bleeding times of 14 mice of the buffer-treated, HGFA-F-treated and IgIV-treated mice were determined after various degrees of bleeding time (FIG. 40). Degree of bleeding was scored randomly by five different persons. Positive control for inducing prolonged bleeding time was heparin at a dose of 10 IE/mouse (n=8). In the reference group, PBS was injected (n=14). Average bleeding time is 368 seconds for PBS-injected mice and 1056 seconds for heparin-injected mice. HGFA-F and IgIV prolonged the bleeding time to, on average, 706 and 765 seconds. According to an unpaired t-test with two-tailed P values, bleeding times in HGFA-F-injected mice and IgIV-injected mice differ significantly from the bleeding time observed with PBS-injected mice (see FIG. 40). P values are 0.013 for HGFA-F and 0.0045 for IgIV, respectively, when compared to the PBS-injected control group. These observations demonstrate a role for crossbeta structure protein conformation in the cascades that result in coagulation and formation of a platelet plug. As depicted in the Examples listed above, fibrin polymerization requires crossbeta structure formation and, as described above, fibrin clot lysis by tPA and plasminogen is inhibited by crossbeta structure-binding compounds. The data obtained with HGFA-F and IgIV demonstrate that anti-coagulant therapeutics based on crossbeta structure-binding compounds or based on compounds that bind to the molecules that bind to crossbeta structure during coagulation and platelet activation are suitable.

### Tables

TABLE 1
Percentage β-sheet, as calculated from CD spectra

| | Incubation time | |
|---|---|---|
| Sample‡ | (weeks) | β-sheet (%)† |
| Aβ(16-22) | | 100 |
| Albumin-glycerald. | 2 | 0 |
| Albumin control | 2 | 0 |
| Albumin-g6p | 2 | 0 |
| Albumin-g6p | 4 | 7 |
| Albumin control | 23 | 0 |
| Albumin-g6p | 23 | 19 |

‡Two-week incubated albumin was from a different batch than four- and 23-week incubated albumin.
†Percentage of amino acid residues in β-sheets are given.

TABLE 2
Correlation between Hb_{A1c} concentrations
and Hb fibril formation in vitro

| Healthy controls | | | Diabetes mellitus patients | | |
|---|---|---|---|---|---|
| sample | [Hb_{A1c}] (%)‡ | Fibers† | sample | [Hb_{A1c}] (%)‡ | Fibers† |
| 1 | 5.6 | − | 1 | 5.5 | − |
| 2 | 5.9 | − | 2 | 5.8 | − |
| 3 | 6.2 | − | 3 | 5.8 | − |
| | | | 4 | 10.7 | − |
| | | | 5 | 11.3 | − |
| | | | 6 | 11.6 | − |
| | | | 7 | 12.4 | + |
| | | | 8 | 12.5 | − |
| | | | 9 | 12.5 | − |
| | | | 10 | 12.6 | + |
| | | | 11 | 12.7 | − |
| | | | 12 | 12.8 | − |
| | | | 13 | 13.3 | + |
| | | | 14 | 13.7 | + |
| | | | 15 | 14.8 | + |
| | | | 16 | 15.3 | + |

‡The Hb_{A1c} concentration is given as a percentage of the total amount of Hb present in erythrocytes of diabetes mellitus patients and of healthy controls. The s.d. is 2.3% of the values given.
†Presence of fibers as determined with TEM.

TABLE 3
cross-β structure-binding compounds

| Congo red | Chrysamine G | Thioflavin T |
|---|---|---|
| 2-(4′-(methylamino)phenyl)-6- | Any other | Glycosaminoglycans |
| methylbenzothiaziole | amyloid-binding | |
| | dye/chemical | |
| Thioflavin S | Styryl dyes | BTA-1 |
| Poly(thiophene acetic acid) | conjugated | |
| | polyeclectrolyte | |
| | PTAA-Li | |

TABLE 4
proteins that bind to and/or interact with cross-β structure
and/or with proteins comprising cross-β structure

| | | |
|---|---|---|
| Tissue-type plasminogen | Finger domain(s) of tPA, | Apolipoprotein E |
| activator | factor XII, fibronectin, HGFA | |
| Factor XII | Plasmin(ogen) | Matrix metalloprotease-1 |
| Fibronectin | 75 kD-neurotrophin receptor | Matrix metalloprotease-2 |
| | (p75NTR) | |
| Hepatocyte growth factor | α2-macroglobulin | Matrix metalloprotease-3 |
| activator | | |
| Serum amyloid P | High molecular weight | Monoclonal antibody |
| component | kininogen | 2C11(F8A6) ^{‡} |
| C1q | Cathepsin K | Monoclonal antibody 4A6(A7) ^{‡} |
| CD36 | Matrix metalloprotease 9 | Monoclonal antibody 2E2(B3) ^{‡} |
| Receptor for advanced | Haem oxygenase-1 | Monoclonal antibody 7H1(C6) ^{‡} |
| glycation endproducts | | |
| Scavenger receptor-A | low-density lipoprotein | Monoclonal antibody 7H2(H2) ^{‡} |
| | receptor-related protein (LRP, | |
| | CD91) | |
| Scavenger receptor-B | DnaK | Monoclonal antibody 7H9(B9) ^{‡} |
| ER chaperone ERp57 | GroEL | Monoclonal antibody 8F2(G7) ^{‡} |
| calreticulin | VEGF165 | Monoclonal antibody 4F4 ^{‡} |
| Monoclonal | Monoclonal conformational | Amyloid oligomer-specific |
| conformational antibody | antibody WO2 (ref. | antibody (ref. (Kayed et al., |
| WO1 (ref. (O'Nuallain and | (O'Nuallain and Wetzel, | 2003)) |
| Wetzel, 2002)) | 2002)) | |
| formyl peptide | α(6)β(1)-integrin | CD47 |
| receptor-like 1 | | |
| Rabbit anti-albumin-AGE | CD40 | apo A-I belonging to small |
| antibody, Aβ-purified^{a}) | | high-density lipoproteins |
| apoJ/clusterin | ten times molar excess | CD40-ligand |
| | PPACK, 10 mM εACA, (100 | |
| | pM-500 nM) tPA²) | |
| macrophage scavenger | broad spectrum (human) | BiP/grp78 |
| receptor CD163 | immunoglobulin G (IgG) | |
| | antibodies (IgIV, IVIg) | |
| ERdj3 | haptoglobin | |

^{‡}Monoclonal antibodies developed in collaboration with the ABC-Hybridoma Facility, Utrecht University, Utrecht, The Netherlands.
^{a})Antigen albumin-AGE and ligand Aβ were sent in to Davids Biotechnologie (Regensburg, Germany); a rabbit was immunized with albumin-AGE, antibodies against a structural epitope were affinity purified using a column with immobilized Aβ.
²)PPACK is Phe-Pro-Arg-chloromethylketone (SEQ-ID 8), εACA is ε-amino caproic acid, tPA is tissue-type plasminogen activator

TABLE 5
Proteins directly or indirectly involved in the crossbeta pathway

| | | |
|---|---|---|
| Monoclonal antibody 4B5 | Heat-shock protein 27 | Heat-shock protein 40 |
| Monoclonal antibody 3H7^{‡} | Nod2 (=CARD15) | Heat-shock protein 70 |
| FEEL-1 | Pentraxin-3 | HDT1 |
| LOX-1 | Serum amyloid A proteins | GroES |
| MD2 | Stabilin-1 | Heat-shock protein 90 |
| FEEL-2 | Stabilin-2 | CD36 and LIMPII |
| | | analogous-I (CLA-1) |
| Low-density Lipoprotein | LPS-binding protein | CD14 |
| C reactive protein | CD45 | Orosomucoid |
| integrins | alpha-1 antitrypsin | apo A-IV-Transthyretin |
| | | complex |
| albumin | Alpha-1 acid glycoprotein | β2-glycoprotein I |
| Lysozyme | Lactoferrin | Megalin |
| Tamm-Horsfall protein | Apolipoprotein E3 | Apolipoprotein E4 |
| Toll-like receptors | Complement receptor | CD11d/CD18 (subunit aD) |
| | CD11b/CD18 (Mac-1, CR3) | |
| CD11b2 | CD11a/CD18 (LFA-1, subunit | CD11c/CD18 (CR4, subunit |
| | aL) | aX) |
| Von Willebrand factor | Myosin | Agrin |
| Perlecan | Hsp60 | b2 integrin subunit |
| proteins that act in the | proteins that act in the | Macrophage receptor with |
| unfolded protein response | endoplasmic reticulum stress | collagenous structure |
| (UPR) pathway of the | response (ESR) pathway of | (MARCO) |
| endoplasmic reticulum (ER) | prokaryotic and eukaryotic cells | |
| of prokaryotic and | | |
| eukaryotic cells | | |
| 20S | HSP16 family members | HSC73 |
| HSC70 | translocation channel protein | 26S proteasome |
| | Sec61p | |
| 19S cap of the proteasome | UDP-glucose:glycoprotein | carboxy-terminus of |
| (PA700) | glucosyl transferase (UGGT) | HSP70-interacting protein |
| | | (CHIP) |
| Pattern Recognition | Derlin-1 | Calnexin |
| Receptors | | |
| Bcl-2 associated athanogene | GRP94 | Endoplasmic reticulum p72 |
| (Bag-1) | | |
| (broad spectrum) (human) | proteins that act in the | The (very) low-density |
| immunoglobulin M (IgM) | endoplasmic reticulum | lipoprotein receptor family |
| antibodies | associated degradation system | |
| | (ERAD) | |
| Fc receptor | | |

^{‡}Monoclonal antibodies developed in collaboration with the ABC-Hybridoma Facility, Utrecht University, Utrecht, The Netherlands.

### REFERENCES

1. Andrade M. A., P. Chacon, J. J. Merelo, and F. Moran (1993). Evaluation of secondary structure of proteins from UV circular dichroism spectra using an unsupervised learning neural network. Protein Eng. 6:383-390.
2. Bouma B., L. M. Kroon-Batenburg, Y. P. Wu, B. Brunjes, G. Posthuma, O. Kranenburg, P. G. de Groot, E. E. Voest, and M. F. Gebbink (2003). Glycation induces formation of amyloid cross-beta structure in albumin. J. Bio. Chem. 278:41810-41819.
3. Dawson P. and S. Kent (2000). Annu. Rev. Biochem. 69:923-960.
4. Dawson P., T. Muir, I. Clark-Lewis, and S. Kent (1994). Science 266:776-779.
5. de Laat H. B., R. H. Derksen, R. T. Urbanus, M. Roest, and P. G. de Groot (2004). beta2-glycoprotein I-dependent lupus anticoagulant highly correlates with thrombosis in the antiphospholipid syndrome. Blood 104:3598-3602.
6. de Laat B., R. H. Derksen, R. T. Urbanus, and P. G. de Groot (2005). IgG antibodies that recognize epitope Gly40-Arg43 in domain I of {beta}2-glycoprotein I cause LAC and their presence correlates strongly with thrombosis. Blood 105(4): 1540-1545.
7. Gebbink M. F., D. Claessen, B. Bouma, L. Dijkhuizen, and H. A. Wosten (2005). Amyloids—a functional coat for microorganisms. Nat. Rev. Microbiol. 3:333-341.
8. Gebbink M. F., G. C. Zondag, G. M. Koningstein, E. Feiken, R. W. Wubbolts, and W. H. Moolenaar (1995). Cell surface expression of receptor protein tyrosine phosphatase RPTP mu is regulated by cell-cell contact. J. Cell. Biol. 131:251-260.
9. Hackeng T. M., J. H. Griffin, and P. E. Dawson (1999). Protein synthesis by native chemical ligation: expanded scope by using straightforward methodology. Proc. Natl. Acad. Sci. U.S.A. 96:10068-10073.
10. Horbach D. A., E. van Oort, M. J. Tempelman, R. H. Derksen, and P. G. de Groot (1998). The prevalence of a non-phospholipid-binding form of beta2-glycoprotein I in human plasma—consequences for the development of anti-beta2-glycoprotein I antibodies. Thromb. Haemost. 80:791-797.
11. Johannessen M., V. Diness, K. Pingel, L. C. Petersen, D. Rao, P. Lioubin, P. O'Hara, and E. Mulvihill (1990). Fibrin affinity and clearance of t-PA deletion and substitution analogues. Thromb. Haemost. 19; 63:54-59.
12. Kayed R., E. Head, J. L. Thompson, T. M. McIntire, S. C. Milton, C. W. Cotman, and C. G. Glabe (2003). Common structure of soluble amyloid oligomers implies common mechanism of pathogenesis. Science 300:486-489.
13. Kranenburg O., B. Bouma, L. M. Kroon-Batenburg, A. Reijerkerk, Y. P. Wu, E. E. Voest, and M. F.-Gebbink (2002). Tissue-type plasminogen activator is a multiligand cross-beta structure-receptor. Curr. Biol. 12:1833-1839.
14. Kranenburg O., L. M. Kroon-Batenburg, A. Reijerkerk, Y. P. Wu, E. E. Voest, and M. F. Gebbink (2003). Recombinant endostatin forms amyloid fibrils that bind and are cytotoxic to murine neuroblastoma cells in vitro. FEBS Lett. 539:149-155.
15. Matsuura E., Y. Igarashi, T. Yasuda, D. A. Triplett, and T. Koike (1994). Anticardiolipin antibodies recognize beta 2-glycoprotein I structure altered by interacting with an oxygen-modified solid phase surface. J. Exp. Med. 179:457-462.
16. Matzinger P. (2002a). An innate sense of danger. Ann. N.Y. Acad. Sci. 961:341-2:341-342.
17. Matzinger P. (2002b). The danger model: a renewed sense of self. Science 296:301-305.
18. O'Nuallain B. and R. Wetzel (2002). Conformational Abs recognizing a generic amyloid fibril epitope. Proc. Natl. Acad. Sci. U.S.A. 99:1485-1490.
19. Subang R., J. S. Levine, A. S. Janoff, S. M. Davidson, T. F. Taraschi, T. Koike, S. R. Minchey, M. Whiteside, M. Tannenbaum, and J. Rauch (2000). Phospholipid-bound beta 2-glycoprotein I induces the production of anti-phospholipid antibodies. J. Autoimmun. 15:21-32.

1. A method for interfering in coagulation of blood and/or platelet activation and/or platelet aggregation and/or fibrinolysis, said method comprising
providing to blood a binding molecule that either binds to a cross-beta structure or to a compound comprising a specific binding partner of cross-beta structure which compound is part of a blood coagulation cascade and/or platelet activation cascade and/or fibrinolytic pathway.

2. (canceled)

3. A method for interfering in coagulation of blood, said method comprising influencing factor XII activation, platelet activation, fibrin polymerization, expression of Tissue Factor, release of Tissue Factor, and/or activation of Tissue Factor.

4. The method according to claim 3, further comprising influencing release of intracellular tissue plasminogen activator (tPA), and/or activation of tPA.

5. The method according to claim 1, wherein coagulation of blood is increased by stimulating expression and/or release of Tissue Factor by nucleated cells.

6. The method according to claim 1, wherein coagulation of blood is increased by counteracting intracellular tPA release by nucleated cells and/or tissue.

7. The method according to claim 6, comprising counteracting local tissue damage and/or local vascular damage.

8. The method according to claim 1, wherein coagulation of blood is increased by activating platelets.

9. The method according to claim 1, wherein factor XII activation, platelet activation, fibrin polymerization, expression of Tissue Factor, release of Tissue Factor and/or activation of Tissue Factor is induced and/or enhanced by providing a cross-beta structure and/or a proteinaceous molecule comprising a cross-beta structure.

10. (canceled)

11. The method according to claim 1, wherein coagulation of blood is decreased by counteracting expression and/or release of Tissue Factor by nucleated cells.

12. The method according to claim 1, wherein coagulation of blood is decreased by inducing and/or enhancing intracellular tPA release by nucleated cells and/or tissue.

13. The method according to claim 1, wherein coagulation of blood is decreased by counteracting fibrin polymerization.

14. The method according to claim 12, comprising inducing and/or enhancing local tissue damage and/or local vascular damage.

15. The method according to claim 1, wherein coagulation of blood is decreased by counteracting platelet activation.

16. The method according to claim 1, wherein factor XII activation, platelet activation, fibrin polymerization, expression of Tissue Factor, release of Tissue Factor and/or activation of Tissue Factor is counteracted by providing a compound that is either capable of specifically binding to a cross-beta structure or to a compound comprising a specific binding partner of cross-beta structure which compound is part of a blood coagulation cascade.

17-18. (canceled)

19. The method according to claim 1, wherein said binding molecule is a bi-specific molecule capable of binding to cross-beta structure as well as to another part of said cross-beta structure or binding to said specific binding partner as well as to another part of said compound which is part of a blood-coagulating cascade.

20. (canceled)

21. The method or according to claim 1, wherein said compound of a blood-coagulating cascade is selected from the group consisting of a platelet, Factor XII, and fibrin.

22-23. (canceled)

24. The method according to claim 1, wherein said specific binding partner is a receptor present on an endothelial cell.

25. The method according to claim 1, wherein said specific binding partner is a receptor which is naturally present on a cell that is exposed to blood.

26. The method according to claim 1, wherein said cross beta structure specific binding partner is selected from the group consisting of CD36, CD91, apoER2′, scavenger receptor A, and scavenger receptor B-I.

27. The method according to claim 1, wherein said specific binding partner is selected from the group consisting of CD36, LRP, scavenger receptor A, scavenger receptor B-I, RAGE, FEEL-1, FEEL-2, SREC-1, LOX-i, stabilin-1, stabilin-2, and CD40.

28. The method according to claim 19, wherein said bi-specific molecule is an antibody.

29. A bi-specific molecule capable of binding to a specific cross-beta structure binding part of a compound which is part of a blood-coagulating cascade as well as to another part of said compound.

30. A bi-specific molecule capable of binding to cross-beta structure as well as to another part of the same cross-beta structure.

31. The bi-specific molecule of claim 29 which is an antibody.

32. A pharmaceutical comprising the bi-specific molecule claim 29.
